(19) United States
(12) Patent Application Publication
(10) Pub. No.: US 2024/0252596 A1
(43) Pub. Date: Aug. 1, 2024

(54) Injectable Polymeric Biopharmaceutical Formulations

(71) Applicant: The Board of Trustees of the Leland Stanford Junior University, Stanford, CA (US)

(72) Inventors: Eric A. Appel, Palo Alto, CA (US); Carolyn Jons, Stanford, CA (US); Alex Prossnitz, Stanford, CA (US)

(21) Appl. No.: 18/405,958

(22) Filed: Jan. 5, 2024

Related U.S. Application Data
(60) Provisional application No. 63/437,239, filed on Jan. 5, 2023.
(60) Provisional application No. 63/532,820, filed on Aug. 15, 2023.

(51) Int. Cl. A61K 38/28; A61K 9/00; A61K 38/22; A61K 39/00; A61K 47/14; A61K 47/26; A61K 47/32; A61K 47/42; C07K 16/06

(52) U.S. Cl. CPC A61K 38/28; A61K 9/0019; A61K 38/22; A61K 47/14; A61K 47/26; A61K 47/32; A61K 47/42; C07K 16/06; A61K 2039/505

(57) ABSTRACT

The present disclosure provides injectable pharmaceutical compositions including suspended particles in a liquid carrier, where the particles of the composition include a biopharmaceutical agent and a polyacrylamide-based copolymer. The inventors have demonstrated that particular polyacrylamide-based copolymers can be used as stabilizing excipients in particle formulations of biopharmaceutical agents, without interacting directly with the biopharmaceutical agent, or altering its pharmacokinetic properties. The pharmaceutical composition can be formulated for injection to a patient. Also provided is a syringe loaded with the injectable pharmaceutical composition, methods of administering via injection a therapeutically effective dose of a biopharmaceutical to a subject in need thereof, and methods of preparing a subject injectable pharmaceutical composition.

### 1. BACKGROUND

Many biopharmaceuticals in aqueous based formulations are prone to irreversible aggregation when exposed to high temperatures and/or agitation and require formulation at low concentrations, careful storage and refrigerated transport (the cold chain) to retain activity over its shelf life. Maintaining the integrity of various biopharmaceuticals prone to aggregation presents a challenge to the pharmaceutical industry, healthcare providers, and people needing treatment with such biopharmaceuticals worldwide. While the mechanism of aggregation can vary between biopharmaceutical agents, their tendency to aggregate at interfaces increases with formulation concentrations, negatively impacting overall formulation stability. Such inherent concentration and/or stability limitations of biopharmaceutical formulations often require that large-volume, low-concentration transfusions of biopharmaceutical therapies are delivered intravenously (IV). However, IV administration of such a formulation places a burden on patients, often requiring lengthy transfusion procedures and access to clinical infrastructure, precluding large numbers of at-risk populations from effective treatments.

Many commercial excipients have been used in trying to overcome challenges associated with formulation of biopharmaceutical compounds. These systems can be limited by their critical micelle concentrations, possible toxicity through oxidative degradation, and undesirable interaction between the excipient and the cargo in the bulk.

Accordingly, there is a need for improved injectable biopharmaceutical formulations.

### 2. SUMMARY

The present disclosure provides injectable pharmaceutical compositions including suspended particles in a liquid carrier. The particles of the composition include a biopharmaceutical agent and a polyacrylamide-based copolymer. The inventors have demonstrated that particular polyacrylamide-based copolymers can be used as stabilizing excipients in particle formulations of biopharmaceutical agents, without interacting directly with the biopharmaceutical agent, or altering its pharmacokinetic properties. The results presented herein indicate that the polyacrylamide-based copolymers of this disclosure can be generally applied to confer a substantial stability benefit to high concentration particle-based compositions of biopharmaceutical agents (such as proteins or peptides) and modify injectability and depot formation properties of the resulting composition.

In some embodiments, the pharmaceutical composition is formulated for injection to a patient at a biopharmaceutical agent concentration that allows for administration of a therapeutically effective dose to be conducted in a low resource setting, in contrast, for example, to compositions which are administered via IV administration. In some embodiments, the pharmaceutical composition is formulated for administration via subcutaneous (SC) injection. In some embodiments, the pharmaceutical composition is formulated for administration via intramuscular (IM) injection. In some embodiments, the injectable pharmaceutical composition is storage stable.

According, in a first aspect, there is provided an injectable pharmaceutical composition comprising:
particles, the particles comprising:
   a biopharmaceutical agent; and
   a polyacrylamide-based copolymer; and
a liquid carrier in which the particles are suspended.

Also provided is a syringe loaded with the injectable pharmaceutical composition, methods of administering via injection a therapeutically effective dose of a biopharmaceutical to a subject in need thereof, and methods of preparing a subject injectable pharmaceutical composition.

### 3. BRIEF DESCRIPTION OF DRAWINGS

FIG. 1, panels a to c, illustrate characterization of MoNi. Panel a depicts two size-exclusion chromatography (SEC) traces of MoNi. Panel b shows a 1H NMR spectrum of MoNi. Panel c depicts a differential scanning calorimetry (DSC) analysis of MoNi at a temperature ramp and cooling rate of 10° C./min showing a glass transition temperature between 130 and 140° C. (top line 1=cooling; lower line 2=heating).

FIG. 2 is a schematic of the spray drying process and formulation of ultra-high-concentration (UHC) protein suspensions.

FIG. 3, panels a-c illustrate injection behavior of exemplary formulations. Panel a illustrates the injection through a 21 G needle and depot forming behavior of formulation 4 (336 mg/mL BSA in sesame oil including 4-acryloylmorpholine_{77%}-N-isopropylacrylamide_{23%} MoNi and trehalose). Panel b illustrates the injection through a 21 G needle and lack of depot forming behavior of formulation 3 (364 mg/mL BSA in sesame oil including trehalose, but no copolymer). Panel c illustrates the injection though a 26 G needle and depot forming behavior of formulation 7 (400 mg/mL BSA in triacetin including the copolymer MoNi and trehalose).

FIG. 4, panels a-d illustrate angular frequency sweep and flow sweep of exemplary formulations. Panel a illustrates the angular frequency sweep for formulation 7 (400 mg/mL BSA in triacetin with MoNi). Panel b illustrates the flow sweep for formulation 7. Panel c illustrates the angular frequency sweep for formulation 6 (400 mg/mL BSA in triacetin without copolymer). Panel d illustrates the flow sweep for formulation 6.

FIG. 5, panels a-c illustrate rheological and stability characterization of ball milled BSA particles. Panel a illustrates angular frequency sweep of i) 400 mg/mL BSA particles resuspended in triacetin; and ii) 400 mg/mL BSA particles containing 5 wt % MoNi resuspended in triacetin. Panel a. iii) Comparative storage modulus of suspensions at 10 rad/sec. Panel b illustrates microscope images of BSA with 5 wt % MoNi particles formed by i) 15 minutes of ball milling; or ii) spray drying. Particles are resuspended in sesame oil for improved imaging. Panel c depicts a SEC trace of fresh BSA control, 15 minute ball milled BSA without MoNi, and 15 minute ball milled BSA with 5 wt % MoNi. PBS with sodium azide is used as an eluent.

FIG. 6, panels a-d illustrate angular frequency sweep and flow sweep of exemplary formulations formed by spray drying. Panel a illustrates the angular frequency sweep for formulation 11 (460 mg/mL BSA in triacetin with MoNi copolymer). Panel b illustrates the flow sweep for formulation 11. Panel c illustrates the angular frequency sweep for formulation 10 (460 mg/mL BSA in triacetin without copolymer). Panel d illustrates the flow sweep for formulation 10. Adding MoNi copolymer results in a 4000× decrease in formulation stiffness.

FIG. 7, panels a-d illustrate rheological and stability characterization of spray dried particles. Panel a illustrates angular frequency sweep of i) 460 mg/mL BSA particles resuspended in triacetin; and ii) 460 mg/mL BSA particles containing 5 wt % MoNi resuspended in triacetin. Panel a. iii) Comparative storage modulus of suspensions at 10 rad/sec. Panel b depicts flow sweep of 460 mg/mL BSA particles resuspended in triacetin and 460 mg/mL BSA particles containing 5 wt % MoNi resuspended in triacetin. Panel c depicts a SEM image of particles (scale bar 5 μm) and image of resultant suspension in triacetin at 460 mg/mL for particles formulated i) without and ii) with 5 wt % MoNi. Panel d depicts a SEC trace of fresh BSA control, spray dried BSA without MoNi, and spray dried BSA with 5 wt % MoNi. PBS with sodium azide is used as an eluent.

FIG. 8, panels a-b illustrate the impact of trehalose and copolymer MoNi on BSA stability after lyophilization (panel a.) and milling (panel b.). Panel a demonstrates that trehalose and MoNi content during lyophilization show little impact on high molecular weight shoulder. Panel b further demonstrates that irrespective of trehalose content, MoNi reduces size of high molecular weight shoulder following 15 minutes of ball milling.

FIG. 9, panels a(i)-a(iii) and b show the results of assessment of exemplary particle compositions for protein stability using SEC. The BSA protein compositions were prepared via spray drying or ball milling. SEC allows for visualization of BSA monomer and dimer peak. Panel a(i): Full SEC traces of fresh, spray dried, and ball milled BSA. Panel a(ii): Dimer peak of fresh, spray dried, and ball milled BSA. Panel a(iii): High molecular weight peak of fresh, spray-dried particles (e.g., particles) with a 100:5 weight ratio of BSA, and ball milled BSA. Panel b: BSA monomer fraction after spray drying and ball milling. These results suggest that spray drying is a gentler process of forming particles than ball milling, but that MoNi is a useful protectant through the ball milling process.

FIG. 10, panels a-c and d(i)-d(ii) illustrate the assessment of injection force and flow rate of injection of exemplary compositions through different gauge needles. Panel a depicts a force sensor connected to a syringe pump was used to quantify the force of injection through standard gauge needles using a 1 mL syringe. Panel b depicts force of injection curves illustrate injection force needed to inject suspensions comprising 460 mg/mL BSA and 23 mg/mL MoNi through 27 G ½ inch needles at varied flow rates. Panel c illustrates that force of injection is linear with flow rate and scales with needle gauge. Panel d(i) shows force of injection curves that illustrate the injection force needed to inject suspensions comprising 460 mg/mL BSA and 23 mg/mL MoNi suspensions through 27 G ½ inch needles and 26 G ½ inch needles at 1 mL/min. Panel d(ii) illustrates that injection force decreases with increased needle gauge. Injection force comparisons to BSA formulations not including MoNi are not included because formulations not including MoNi are not injectable through 26 or 27 G needles.

FIG. 11, panels a-c illustrate injection force studies of exemplary compositions through different gauge needles and after vertical storage. Panel a(i) further illustrates that force of injection is linear with flow rate, and ii) scales with needle gauge. Panel b shows a picture of syringe used for vertical storage of exemplary composition for up to 120 hours. Panel c depicts the injection force of a 460 mg/mL BSA with 5% MoNi in triacetin composition at day 0 and day 35 using a 26 G ½ inch needle and a flow rate of 1 mL/min.

FIG. 12, panels a-b shows the results of stressed aging test (30 minutes at 60° C.) on exemplary BSA formulations. Panel a depicts SEC traces of BSA formulations following stressed aging. Panel b illustrates the corresponding BSA monomer fraction after stressed aging conditions. BSA in solution in PBS at 20 mg/mL saw significant aggregation. BSA suspensions in triacetin saw minimal aggregation. BSA suspension in triacetin containing 5 wt % MoNi in the particles of the suspension had no high molecular weight peaks beyond the dimer peak at 31 minutes indicating good stability. BSA in triacetin without MoNi in the particles shows a small high molecular weight peak at 16 minutes, but relatively good stability.

FIG. 13, panels a(i)-a(iii) show the assessment of storage stability of exemplary composition (460 mg/mL BSA formulations in triacetin with and without 5 wt % MoNi in the particles of the suspension) stored for 48 hours at 4 C, 25 C, and 37 C. Panel a(i) depicts full SEC traces of formulations after 48 hours. Panel a(ii) depicts the dimer peak of all formulations after 48 hours. Panel a(iii) depicts a high molecular weight peak of all formulations after 48 hours. Generally, all formulations show good stability after 48 hours with the majority of light scattering signal coming from the monomer peak. Samples containing MoNi in the particles on average show smaller dimer and high molecular weight peaks than samples without MoNi suggesting improved storage stability.

FIG. 14, panels a(i)-a(iii) show the assessment of storage stability of exemplary composition (460 mg/mL BSA formulations in triacetin with and without 5 wt % MoNi in the particles of the suspension) stored for 120 hours at 4° C., 25° C., and 37° C. Panel a(i) depicts full SEC traces of exemplary formulations after 120 hours. Panel a(ii) shows dimer peak of all formulations after 120 hours. Panel a(iii) shows a high molecular weight peak of all formulations after 120 hours. BSA formulations containing MoNi in the particles stored for 120 hours in a syringe showed no particle settling. Generally, all formulations show good stability after 120 hours with the majority of light scattering signal coming from the monomer peak. Samples containing MoNi in the particles on average show smaller high molecular weight peaks than samples without MoNi suggesting improved storage stability.

FIG. 15 shows the results of a centrifuge study to evaluate suspension stability in the following media from left to right: triacetin alone; triacetin with 20% benzyl benzoate; triacetin with 20% benzyl alcohol; and triacetin with 20% safflower oil.

FIG. 16 illustrates viscometer measurements for various liquid carriers. This graph illustrates how the viscosity of a higher viscosity non-solvent (propylene glycol (PG) or triacetin (T)) can be reduced by adding a lower viscosity additive, such as benzyl alcohol (BA).

FIG. 17, panels a-d show that injection force measurements (1 mL/min through a 26 G, ½ inch needle) show a significant reduction in injection force when DMAc is used in combination with triacetin as a non-solvent. Panel a shows the plateau injection force for triacetin suspensions (100%) vs. suspensions in various blends of triacetin: DMAc and triacetin: DMAc: BA. Panel b shows the injection force of triacetin suspensions vs DMAc content. Panel c shows the injection force of triacetin suspensions (100%) vs suspensions in various blends of triacetin: DMAc and triacetin: DMAc: BA over time. Panel d shows that the addition of polymer MoNi affects the consistency of BSA powder dispersed in DMAc. The left image of panel d illustrates a formulation having 450 mg/mL BSA DMAc including MoNi. The right image of panel d illustrates a formulation having 450 mg/mL BSA in DMAc without the addition of MoNi.

FIG. 18, panels a-c show the results of in-vivo administration to mice of 4 mg BSA by either 200 injections of 20 mg/mL BSA in PBS (bolus injection), or 9 μL injections of 450 mg/mL BSA in triacetin (high concentration paste/triacetin paste) (N≥3 per group). Panel a depicts the fluorescent signal following in-vivo administration of 4 mg BSA by either 200 injections of 20 mg/mL BSA in PBS (bolus injection), or 9 μL injections of 450 mg/mL BSA in triacetin (high concentration paste/triacetin paste). Panel b shows comparative administration volume of high concentration paste and 20 mg/mL bolus injection. Panel c shows comparative half-life of BSA subcutaneous absorption from high concentration paste and bolus administration.

FIG. 19, panels a-b show the results of in vivo administration of 9 μL of 450 mg/mL BSA in a 70:30 triacetin: DMAc suspension. Panel a is a fluorescence decay curve associated with the in-vivo administration of 4 mg of BSA by 9 μL injections of 450 mg/mL BSA in 70:30 triacetin: DMAc. Panel b shows the half-life of subcutaneous absorption of a paste in 100% triacetin; a bolus injection (20 mg/mL BSA in PBS); and a suspension in 70:30 triacetin: DMAc (N≥3 per group).

FIG. 20, panels a-b illustrate the products obtained after spray drying formulations containing polysorbate 80 and Pluronic L-61. Panel a shows images of falcon tubes containing comparative products recovered from spray drying equal mass of BSA with polysorbate 80 (left tube) and Pluronic L-61 (right tube). Panel b shows the spray dryer glass component after spray drying Pluronic L-61. As evident in the image, spray drying Pluronic L-61 results in a tacky powder that sticks to the spray dryer glass components, thus resulting in a poor yield of the spray dried product (see panel a, right tube, showing little recovered product).

FIG. 21, panels a-c illustrate SEC traces for fresh BSA and BSA (Fresh BSA Standard) spray dried with MoNi (BSA_MoNi), polysorbate 80 (BSA_Tw80), and no additive (BSA_NoMoNi). Panel a shows for all spray dried samples, the monomer and dimer peaks appear similar. Panel b shows differences between the spray dried samples for the high molecular weight aggregate peaks. Panel c shows the Area fraction of the high molecular weight peak is smallest for fresh BSA standard, followed by BSA spray dried in MoNi, BSA spray dried with polysorbate 80, and finally BSA spray dried without an additive.

FIG. 22, panels a-c illustrate SEC analysis of high molecular weight aggregates in aged protein pastes made with BSA, BSA with MoNi, and BSA with polysorbate 80. BSA aged at 20 mg/mL in PBS was used as a control. All samples were aged at 60° C. for 60 minutes. Panel a shows the full SEC traces, and panel b shows differences between the samples for the high molecular weight aggregate peaks. Panel c shows the Area percentage of the high molecular weight peaks for each sample.

FIG. 23, panels a-c show injection force experiments (1 mL/min through a 26 G ½ inch needle) with 450 mg/mL BSA suspensions in triacetin. The BSA has been spray dried with either polysorbate 80 (Tween 80) or MoNi at equal molar amounts (7.14 μmol per gram of BSA). Panel a shows the injection force (N) over time (seconds). Panel b shows the plateau injection force for the formulation including MoNi vs polysorbate 80 (Tween 80). Panel c depicts SEM of particle morphology for i) BSA MoNi and ii) BSA polysorbate 80 (Tween 80) particles (scale bars are 10 μm).

FIG. 24, panels a-b: Panel a depicts comparative protein concentration as predicted from density measurements and measured via nanodrop from a known slurry volume. Panel b illustrates injection force as a function of concentration for BSA particles (with mol % matched MoNi or Tween 80) in 70 Triacetin: 30 DMAc. Injection force as a function of particle concentration is fit to a particle jamming model.

FIG. 25, panels a-b show the SEM images of the BSA microparticle formulations. Panel a shows the SEM image of the 004A formulation (smooth particles≤5 μm). Panel b shows the SEM image of the 004B formulation (smooth particles≤5 μm).

FIG. 26, panels a-b show photographic images of the BSA microparticle formulations suspended in triacetin and loaded in 1 mL Schott syringes. Panel a shows a photographic image of the syringe loaded with 004A (480 mg/mL) formulation suspended in triacetin. Panel b shows a photographic image of the syringe loaded with 004B (458 mg/mL) formulation suspended in triacetin.

FIG. 27, panels a-b are graphs of the injection force (N) of the BSA microparticle suspensions over 5 seconds at various flow rates. Panel a shows injection force (N) over 5 seconds for BSA microparticle suspension 004A (480 mg/mL BSA) in triacetin injected at 2 mL/min, 4 mL/min and 6 mL/min from a 1 mL Schott syringe (27 G thin-walled needle and V9519 coated plunger). Panel b shows injection force (N) over 5 seconds for BSA microparticle suspension 004B (458 mg/mL BSA) in triacetin injected at 2 mL/min, 4 mL/min and 6 mL/min from a 1 mL Schott syringe (27 G thin-walled needle and V9519 coated plunger).

FIG. 28 illustrates the injection force measurements of 004A (480 mg/mL BSA) and 004B (458 mg/mL) microparticle suspensions in triacetin injected at different flow rates from 1 mL Schott syringes (27 G thin-walled needle and V9519 coated plunger). This graph shows that injection force (N) for suspensions of 004A and 004B in triacetin is linear with flow rate.

FIG. 29, panels a-g illustrate that high concentration suspension technology can be effectively used to deliver hIgG. Panel a depicts SEM images of spray dried IgG with MoNi particle morphology (scale bars are 10 μm). Panel b depicts SEC trace of fresh hIgG control, spray dried hIgG without MoNi, spray dried hIgG with 5 wt % MoNi, spray dried hIgG with 25 wt % trehalose and 5 wt % MoNi. PBS with sodium azide is used as an eluent. Panel c shows graph of injection force as a function of concentration for hIgG microparticles with MoNi in triacetin. Injection force as a function of particle concentration is fit to a particle jamming model. Panel d depicts injection force for 350 mg/mL hIgG, 5 wt % MoNi suspensions at 1 mL/min through 26 G ½ inch needles with DMAc, benzyl alcohol (BA) and benzyl benzoate (BB) non-solvent additives. Panel e(i) shows representative IVIS images of mice demonstrating subcutaneous absorption of fluorescently-tagged hIgG administered via a PBS bolus injection or a high concentration protein suspension. Panel e(ii) depicts a graph of fluorescent signal in the subcutaneous space is fit to a single phase exponential decay mode to identify half-life of subcutaneous absorption. Panel f depicts a graph of comparative half-life of subcutaneous absorption for hIgG administered via PBS bolus injection or UHC protein suspensions formulated with triacetin. Panel g depicts a graph of comparative volume of administration for bolus injection and high concentration protein suspension. 1

FIG. 30, panels a-b. Panel a, i) shows full SEC traces of fresh hIgG control, spray dried hIgG with no additives, spray dried hIgG with 1×MoNi (5 wt %), spray dried hIgG with 2×MoNi (10 wt %), spray dried hIgG with 25 wt % trehalose and 5 wt % MoNi, and spray dried IgG with 30 wt % Trehalose. Panel a, ii) shows SEC trace of only high molecular weight aggregate. PBS with sodium azide is used as an eluent. Panel b shows a graph of percent change in hIgG aggregates due to spray drying, as a percent of total protein content. The baseline for determining percent change was determined by subtracting the starting aggregate percent of the fresh IgG control (3.9%) from all samples.

FIG. 31, panels a-b. Panel a depicts injection force curves for injection of 300 mg/mL IgG, 5 wt % MoNi suspensions at 1 mL/min through BD Insulin syringes (28 G, 12.7 mm needle) with triacetin as a non-solvent, and panel b shows a graph of corresponding plateau injection force.

FIG. 32, panels a-b show SEC traces of insulin before and after spray drying. Panel a shows the insulin SEC Trace of spray dried insulin and pre-spray dried insulin over 22 minutes. Panel b is a zoomed in portion of minutes 14-17 of the SEC trace in panel a.

FIG. 33, panels a-b show SEC traces of pramlintide before and after spray drying. (Panel a) shows the pramlintide SEC Trace of spray dried pramlintide and pre-spray dried pramlintide over 20 minutes. (Panel b) is a zoomed in portion of minutes 14-17 of the SEC trace in panel a.

FIG. 34 shows SEC trace of pramlintide (“pram”), insulin, and controls before and after spray drying.

FIG. 35 panels a-b. SEM images of insulin and pramlintide spray dried particles resuspended in triacetin (panel a) and trehalose and MoNi (95:5) particles (panel b). Particles in panel b do not contain either insulin or pramlintide, so protein may affect particle morphology.

FIG. 36 shows insulin and pramlintide spray dried particles resuspended in triacetin at a solids content of 350 mg/mL. Particle suspension flows like a liquid.

FIG. 37, panels a and b show injection force of insulin pramlintide coformulation (350 mg/mL solids) in triacetin. Injection measurements were performed using a 1 mL syringe at 1 mL/min through a 26 G ½ inch needle.

FIG. 38 is an image depicting the insulin pramlintide coformulation redissolved in cell grade water.

FIG. 39, panels a-c shows graphs of the syringe force profiles for exemplary BSA formulations with various liquid carriers. Panel a depicts the syringe force profile for formulation 12 (benzyl benzoate as liquid carrier). Panel b depicts the syringe force profile for formulation 13 (Miglyol 840 as liquid carrier). Panel c depicts the syringe force profile for formulation 16 (ethyl oleate as liquid carrier).

FIG. 40 is a graph showing the syringe force profile for exemplary BSA formulation 17 (triacetin:DMAc (75:25) as liquid carrier).

### 4. DETAILED DESCRIPTION

As summarized above, the present disclosure provides pharmaceutical compositions including particles suspended in a liquid carrier. The particles generally include a biopharmaceutical active agent to be administered to a subject in need thereof, and a polyacrylamide-based copolymer that confers a benefit upon the resulting composition, such as a stability benefit, and/or provides for formulation of the biopharmaceutical agent at a concentration suitable for administration via injection. Thus, the pharmaceutical compositions of this disclosure can be referred to as injectable pharmaceutical compositions.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### 4.1. Particles

The pharmaceutical compositions can include particles including a biopharmaceutical agent and a polyacrylamide-based copolymer.

The particles can be prepared (e.g., as descried herein) from a precursor composition including the biopharmaceutical agent, polyacrylamide-based copolymer, and one or more optional excipients, where the composition can be subjected to a variety of processes for preparing a microparticulate form of the precursor composition, including processes such as lyophilization, spray-drying, freeze-drying, spray-freeze drying, milling, or a combination thereof. Once the particles are prepared prior to suspension in a liquid.

The particles size can depend on the particular composition, and selected method of preparation. Characterization of particle(s) size and/or dispersity in a composition can be performed using methods such as optical microscopy imaging.

The particles can include microparticles and/or nanoparticles.

In some embodiments, the particles have a mean diameter of 100 microns or less. In some embodiments, the particles have a mean diameter of 95 microns or less, for example, 90, 85, 80, 75, 70, 65, 60, 55, 50, 45, 40, 35, 30, 25, 20, 15, 10, or 5 microns or less.

In some embodiments, the particles have a mean diameter of from 0.01 to 100 microns, such as from 0.01 to 100 microns, from 0.01 to 50 microns, from 0.01 to 20 microns, from 0.01 to 10 microns, or from 0.01 to 1 micron.

In some embodiments, the particles have a mean diameter of from 0.1 to 100 microns, such as from 0.1 to 50 microns, or for example, a mean diameter from 0.1 to 20 micron, 0.1 to 10 microns, 10 to 20 microns, 20 to 30 microns, 30 to 40 microns, 40 to 50 microns, 50 to 60 microns, 60 to 70 microns, 70 to 80 microns, 80 to 90 microns, or 90 to 100 microns.

In some embodiments, the particles have a mean diameter of from 1 to 100 microns, for example, a mean diameter from 1 to 5 microns, 5 to 10 microns, 10 to 15 microns, 15 to 20 microns, 20 to 25 microns, 25 to 30 microns, 30 to 35 microns, 35 to 40 microns, 40 to 45 microns, 45 to 50 microns, 50 to 55 microns, 55 to 60 microns, 60 to 65 microns, 65 to 70 microns, 70 to 75 microns, 75 to 80 microns, 80 to 85 microns, 85 to 90 microns, 90 to 95 microns and 95 to 100 microns.

In some embodiments, the particles have a mean diameter of from 1 to 50 microns. In some embodiments, the particles have a mean diameter of from 10-20 microns, for example, a mean diameter of 10, 11, 12, 13, 14, 15, 16, 17, 18, 19 or 20 microns.

The particles can be suspended in a liquid carrier (e.g., as described herein). In some embodiments, a particle composition is provided separately from a liquid carrier and can be formulated as a suspension in the liquid carrier prior to use. In some embodiments, an amount of the particle composition is provided already formulated as a suspension in the liquid carrier at a suitable amount and concentration ready for use.

### 4.1.1. Biopharmaceutical Agents

The particles of the pharmaceutical composition also include a biopharmaceutical agent. In some embodiments, the amount of biopharmaceutical agent present in the pharmaceutical composition is sufficient to provide a unit dose suitable for administration via injection. In some embodiments, the particles include a single biopharmaceutical agent. In some embodiments, the particles include a co-formulation of two or more biopharmaceutical agents.

In some embodiments, the particles are formulated at a weight percent of the biopharmaceutical agent in a volume suitable for injection (e.g., SC or IM) of a unit dose to a patient in need thereof. In some embodiments, the biopharmaceutical agent has a high MW, e.g., a large biologic. In some embodiments, a high MW biopharmaceutical agent is one having a MW of 20 kDa or more, such as 30 kDa or more, 40 kDa or more, 50 kDa or more, 60 kDa or more, 70 kDa or more, 80 kDa or more, 90 kDa or more, or 100 kDa or more.

In some embodiments, the particles include about 20 wt % or more of the biopharmaceutical agent, for example, about 25 wt % or more, about 30 wt % or more, about 40 wt % or more, about 50 wt % or more, about 60 wt % or more, about 65 wt % or more, about 70 wt % or more, about 75 wt % or more, about 80 wt % or more, about 85 wt % or more, about 90 wt % or more, about 95 wt % or more of the biopharmaceutical agent. In some embodiments, the particles includes about 90 wt % to 95 wt % of the biopharmaceutical agent.

In some embodiments, the particles are formulated to contain a therapeutically effective amount of a biopharmaceutical agent, where the biopharmaceutical agent has a MW of 10 kDa or less, such as 5 kDa or less. In some embodiments, the particles include no more than 20 wt % of the biopharmaceutical agent, for example, no more than 15 wt %, no more than 10 wt %, no more than 9 wt %, no more than 8 wt %, no more than 7 wt %, no more than 6 wt %, no more than 5 wt %, no more than 4 wt %, no more than 3 wt %, no more than 2 wt %, or no more than 1 wt % of the biopharmaceutical agent. In some embodiments, the particles include about 1 wt % to 20 wt % of the biopharmaceutical agent, for example about 1 wt % to about 10 wt %. In some embodiments, the particles include 1 wt % to 20 wt % of the biopharmaceutical agent, for example 1 wt % to 10 wt %. In such cases, the remainder of the particle composition can be composed of the polyacrylamide-based copolymer, and/or one or more optional components such as a stabilizing agent (e.g., as described herein).

In some embodiments, the biopharmaceutical agent is a polypeptide. In some embodiments, the polypeptide is susceptible to aggregation in an aqueous medium. In some embodiments, the polypeptide is a protein. In some embodiments, the polypeptide is a peptide.

In some embodiments, the biopharmaceutical agent is selected from antibodies and fragments thereof, chimeric fusion proteins, cytokines, chemokines, hormones, vaccine antigens, cancer antigens, adjuvants, and combinations thereof. In some embodiments, the biopharmaceutical agent is a polypeptide.

In some embodiments, the biopharmaceutical agent is an antibody.

The term “antibody” is used herein in its broadest sense and includes certain types of immunoglobulin molecules comprising one or more antigen-binding domains that specifically bind to an antigen or epitope. An antibody specifically includes, but is not limited to, full length antibodies (e.g., intact immunoglobulins), antibody fragments, monoclonal antibodies, polyclonal antibodies, multispecific antibodies (e.g., bispecific antibodies), humanized fully human antibodies, chimeric antibodies, and single domain antibodies.

In some embodiments, the biopharmaceutical agent is a monoclonal antibody or fragment thereof. In some embodiments, the antibody is a monoclonal antibody. In some embodiments, the antibody is monospecific, i.e., binds a single antigen. In some embodiments, the monoclonal antibody is an IgG1 antibody, an IgG2 antibody, an IgG3 antibody, an IgG4 antibody, an IgM antibody, and IgA antibody, or any hybrid thereof.

In some embodiments, the antibody is a chimeric antibody. The term “chimeric antibody” refers to an antibody that contains one or more regions from one antibody and one or more regions from one or more other antibodies.

In some embodiments, the antibody is multispecific, i.e., binds multiple antigens, e.g., a bispecific antibody.

In some embodiments, the antibody is an antibody fragment. An “antibody fragment” includes a portion of an intact antibody, such as the antigen-binding or variable region of an intact antibody. Antibody fragments suitable for use in the present compositions include, for example, Fv fragments, Fab fragments, F(ab′)₂ fragments, Fab′ fragments, scFv (sFv) fragments, and scFv-Fc fragments.

In some embodiments, the biopharmaceutical agent is a chimeric protein. In some embodiments, the chimeric protein is a recombinant fusion protein. The chimeric protein can include two or more domains connected via optional linkers or spacers. In some embodiments, the chimeric protein comprises an antibody fragment, such as an Fc or fragment or variant thereof. In some embodiments, the chimeric protein comprises an antibody fragment fused to a protein domain, e.g., a protein domain that specifically binds to a therapeutic target of a biopharmaceutical agent. In some embodiments, the chimeric protein is an Fc fusion protein.

In some embodiments, the antibody is a single domain antibody, e.g., a camelid antibody, or nanobody.

In some embodiments, the antibody is an antibody-drug conjugate, e.g., an antibody conjugated to one or more heterologous molecule(s). The heterologous molecule can be a small molecule (e.g., an organic compound with a molecular weight of less than 1000, 900, 800, 700, 600, or 500 Daltons). In some embodiments, the heterologous molecule is a cytotoxic agent, a chemotherapeutic agent, or a cytostatic agent.

In some embodiments, the antibody is a bispecific antibody immunoconjugate.

In some embodiments, the antibody is an antibody fragment. In some embodiments, the antibody includes one or more single-chain variable fragments (scFv) of a monoclonal antibody. In some embodiments, the monoclonal antibody is a humanized antibody, a human antibody, a murine antibody, or a chimeric (mouse/human) antibody.

The antibody can be targeted to a variety of target proteins, e.g., a therapeutic target protein.

In some embodiments, the antibody has a molecular weight of about 100 kDa to about 200 kDa, for example, about 120 kDa to about 180 kDa. In some embodiments, the antibody has a molecular weight of about 150 kDa. In some embodiments, the antibody has a molecular weight of 100 kDa to 200 kDa, for example, 120 kDa to 180 kDa. In some embodiments, the antibody has a molecular weight of 150 kDa. In some embodiments, the antibody has a molecular weight of 100 kDa or less, such as 40 kDa to 80 kDa, for example, about 50 kDa.

In some embodiments, the particles include 5 wt % or more of the antibody, for example, 10 wt % or more, 20 wt % or more, 30 wt % or more, 40 wt % or more, 50 wt % or more, 60 wt % or more, 70 wt % or more, 80 wt % or more, 90% or more, or 95% or more of the antibody. In some embodiments, the composition includes 95 wt % or less, 90 wt % or less, or 80 wt % or less of the antibody. In some embodiments, the antibody is a monoclonal antibody or fragment thereof.

In some embodiments, the particles includes about 60 wt % to about 99 wt % of the antibody, for example, about 65 wt % to about 95 wt %, about 70 wt % to about 95 wt %, about 75 wt % to about 95 wt %, about 80 wt % to about 95 wt %, about 85 wt % to about 95 wt %, or about 90 wt % to about 95 wt % of the antibody. In some embodiments, the particles include about 90 wt %, or about 95 wt % of the antibody. In some embodiments, the particles include 60 wt % to 99 wt % of the antibody, for example, 65 wt % to 95 wt %, 70 wt % to 95 wt %, 75 wt % to 95 wt %, 80 wt % to 95 wt %, 85 wt % to 95 wt %, or 90 wt % to 95 wt % of the antibody. In some embodiments, the antibody is a monoclonal antibody or fragment thereof.

In some embodiments, the biopharmaceutical agent is insulin.

The term “insulin” refers to a hormone produced by the beta cells in the pancreatic islets that regulates the amount of glucose in the blood. Many eukaryotes, including humans, primates, pigs, cows, cats, dogs, and rodents, produce insulin. Thus, “insulin,” as used herein, includes insulin produced by humans, and analogs thereof, as well as insulin, and analogs thereof, produced by other eukaryotes, including, but not limited to, primates, pigs, cows, cats, dogs, and rodents, and also includes recombinant, purified or synthetic insulin or insulin analogs having similar function and structure, unless otherwise specified. The human insulin protein consists of 51 amino acids and has a molecular weight of approximately 5.8 kilodalton (kDa). Human insulin is a heterodimer of an A-chain and a B-chain that are connected by disulfide bonds.

Insulin can be isolated from the pancreatic islets extracts of an animal that produces insulin or expressed recombinantly in a suitable expression system such as E. coli, yeast, insect cells, and mammalian cells (e.g., Chinese hamster ovary (CHO) cells). Depending upon their specific pharmacokinetics and pharmacodynamics (PK/PD) properties (e.g., duration of action, maximum concentration observed (Cₘₐₓ), time-to-onset, area under the curve (AUC)), insulin can be further characterized as a rapid-acting insulin, a short-acting insulin, an intermediate-acting insulin, a long-acting insulin, and a pre-mixed insulin.

Insulin also includes monomeric and oligomeric forms, such as dimeric and hexameric forms. Insulin can exist as a monomer as it circulates in the plasma, and it also binds to its receptor while in a monomeric form. Insulin formulations (or insulin analog formulations) containing a predominance of protein molecules in the form of monomers and dimers ordinarily have a strong tendency to aggregate and form inactive fibrils. Insulin hexamers are too large to be absorbed, and so hexameric insulin formulations must disassemble into dimers or monomers before the insulin can be absorbed and function in the body. The active form of insulin in the blood stream is the monomeric form.

In some embodiments, the particles include about 0.5 wt % to about 20 wt %, about 1 wt % to about 20 wt %, about 1 wt % to about 15 wt %, about 1 wt % to about 10 wt %, about 1 wt % to about 9 wt %, about 1 wt % to about 8 wt % of the insulin or the analog thereof. In some embodiments, the particles include about 7 wt %, about 8 wt %, about 9 wt %, or about 10 wt % of the insulin or the analog thereof. In some embodiments, the particles include 1 wt % to 20 wt %, 1 wt % to 15 wt %, 1 wt % to 10 wt %, 1 wt % to 9 wt %, or 1 wt % to 8 wt % of the insulin or the analog thereof. In some embodiments, the particles include 7 wt %, 8 wt %, 9 wt %, or 10 wt % of the insulin or the analog thereof.

In some embodiments, the insulin or the analog thereof is selected from insulin lispro, HUMALOG® (fast-acting insulin lispro), insulin glargine, LANTUS® (insulin glargine), insulin detemir, LEVEMIR® (insulin detemir), ACTRAPID® (fast-acting human insulin), modern insulin, NOVORAPID® (insulin aspart), VELOSULIN® (human insulin), HUMULIN® M3 (a mixture of soluble insulin and isophane insulin called biphasic isophane insulin), HYPURIN® (neutral bovine insulin), INSUMAN® (recombinant human insulin), INSULATARD® (long-acting isophane human insulin), MIXTARD® 30 (a mixture of 30% soluble insulin and 70% isophane insulin), MIXTARD® 40 (a mixture of 40% soluble insulin and 60% isophane insulin), MIXTARD® 50 (a mixture of 50% soluble insulin and 50% isophane insulin), insulin aspart, insulin glulisine, insulin isophane, insulin degludec, insulin icodec, insulin zinc extended, NOVOLIN® R (human insulin), HUMULIN® R (human insulin), HUMULIN® R regular U-500 (concentrated regular insulin), NOVOLIN® N (intermediate-acting human insulin), HUMULIN® N (intermediate-acting human insulin), RELION® (over-the-counter brand of NOVOLIN® R, NOVOLIN® N, and NOVOLIN® 70/30), AFREZZA® (rapid-acting inhaled insulin), HUMULIN® 70/30 (a mixture of 70% human insulin isophane suspension and 30% human insulin injection), NOVOLIN® 70/30 (a mixture of 70% NPH, human insulin isophane suspension and 30% regular, human insulin injection), NOVOLOG® 70/30 (a mixture of 70% insulin aspart protamine suspension and 30% insulin aspart injection), HUMULIN® 50/50 (a mixture of 50% human insulin isophane suspension and 50% human insulin injection), HUMALOG® Mix 75/25 (a mixture of 75% insulin lispro protamine suspension and 25% insulin lispro injection), insulin aspart protamine-insulin aspart, insulin lispro protamine-insulin lispro, human insulin NPH-human insulin regular, insulin degludec-insulin aspart, and combinations thereof. In some embodiments, the insulin or the analog thereof is a human insulin or a recombinant human insulin. In some embodiments, the insulin or the analog thereof is a non-human (e.g., primate, pig, cow, cat, dog, or rodent) insulin or a recombinant non-human insulin. In some embodiments, the insulin or the analog thereof is a purified or synthetic insulin. In some embodiments, the insulin or the analog thereof is selected from a rapid-acting insulin, a short-acting insulin, an intermediate-acting insulin, a long-acting insulin, and a pre-mixed insulin. In some embodiments, the insulin or the analog thereof is insulin lispro. In some embodiments, the insulin or the analog thereof is insulin aspart. In some embodiments, the insulin, or an analog thereof, is recombinant human insulin.

In some embodiments, the biopharmaceutical agent is a peptide or peptide analogue. The formulation approaches described herein should be particularly useful in preparing storage stable injectable pharmaceutical compositions of a variety of therapeutic peptides, including but not limited to glucagon, glucagon-like peptide-1 (GLP-1), glucose-dependent insulinotropic polypeptide (GIP), a GLP-1 receptor agonist, GLP-2, adrenocorticotropic hormone (ACTH), leuprolide, hirudin, insulin, pramlintide, exendins, exenatide, gastric inhibitory peptide, calcitonin, calcitonin gene related peptide, amylin, adrenomedullin, angiotensin, an immunogenic peptide (e.g., a peptide or peptide complex derived from a virus, a bacterium, or any prokaryotic or eukaryotic organism or cell thereof), and the like, and analogues thereof.

In some embodiments, the biopharmaceutical agent is glucagon peptide, glucagon analog, glucagon mimetic, or salt thereof.

### 4.1.1.1 Additional Active Agents

The particles of the present disclosure can include a combination of biopharmaceutical agents and one or more additional therapeutic agents. In some embodiments, the one or more additional therapeutic agents are useful for treating a disease or condition that is targeted by biopharmaceutical agent. In some embodiments, administration of the agents from the pharmaceutical composition can provide for delivery to a cell in the subject's body so as to exert their biological or therapeutic effect at the same time. In some embodiments, the agents in the particles are co-formulated to provide pharmacokinetic profiles that are substantially similar.

In some embodiments, the one or more additional therapeutic agent is an active agent that provides a synergistic effect with the biopharmaceutical agent. In some embodiments, the additional therapeutic agent is a small molecule drug.

In some embodiments, the stable formulations and particles used in accordance with the present disclosure include co-formulations or mixtures of the types of biopharmaceutical agent described herein, such as at least one peptide, at least one small molecule, and combinations thereof.

In some embodiments, the particles are co-formulated to contain a first biopharmaceutical agent that is a protein, and a second biopharmaceutical agent that is a peptide. In some embodiments, the particles are co-formulated to contain first and second biopharmaceutical agents that are peptides. In some embodiments, the particles are co-formulated to contain a first biopharmaceutical agent that is a peptide hormone or analog thereof, and a second therapeutic agent that is a small molecule. In some embodiments, the second therapeutic agent is a steroid. In some embodiments, the co-formulation is a mixture of a first batch of particles containing a biopharmaceutical that is a protein, and a second batch of particles containing a biopharmaceutical that is a peptide.

In some embodiments, the particles are co-formulated to contain a first biopharmaceutical agent that is insulin, and a second biopharmaceutical agent that is a peptide. In some embodiments, the co-formulation is a mixture of a first batch of particles containing insulin, and a second batch of particles containing a peptide. In some embodiments, the peptide is pramlintide.

The amounts of active agent(s) that may be combined with each other and the carrier materials of the particles to produce a dosage form will vary depending upon the subject in need and the particular mode of administration.

### 4.1.2. Polyacrylamide-Based Copolymers

The particles provided in the present disclosure can include a polyacrylamide-based copolymer that is formulated with the biopharmaceutical agent of interest.

Polyacrylamide-based copolymers of interest and that can be used in the particles and particle suspensions described herein include those described in International Publication Nos. WO 2021/211976 and WO 2023/230046, the disclosures of which are herein incorporated by reference.

In some embodiments, the polyacrylamide-based copolymer contains a water-soluble carrier monomer and a functional dopant monomer. In some embodiments, the polyacrylamide-based copolymer is amphiphilic.

In some embodiments, the polyacrylamide-based copolymer includes a non-ionic water-soluble acrylamide monomer and a functional dopant monomer. In some embodiments, the polyacrylamide-based copolymer further includes a functional acrylamide dopant monomer selected from a hydrophobic functional acrylamide dopant monomer, an aromatic functional acrylamide dopant monomer, a hydrogen-bonding functional acrylamide dopant monomer, and an ionic functional acrylamide dopant monomer.

The polyacrylamide-based copolymers of the present disclosure contain a water-soluble carrier monomer. In some embodiments, the water-soluble carrier monomer is non-ionic. In some embodiments, the water-soluble carrier monomer is selected from N-(3-methoxypropoyl)acrylamide (MPAM), 4-acryloylmorpholine (MORPH), N,N-dimethylacrylamide (DMA), N-hydroxyethyl acrylamide (HEAM), and acrylamide (AM), or combinations thereof. In some embodiments, the water-soluble carrier monomer is selected from MPAM and MORPH. In some embodiments, the water-soluble carrier monomer is N-(3-methoxypropoyl)acrylamide (MPAM). In some embodiments, the water-soluble carrier monomer is 4-acryloylmorpholine (MORPH). In some embodiments, the water-soluble carrier monomer is N,N-dimethylacrylamide (DMA). In some embodiments, the water-soluble carrier monomer is N-hydroxyethylacrylamide (HEAM). In some embodiments, the water-soluble carrier monomer is acrylamide (AM). In some embodiments, the copolymer includes a water-soluble carrier monomer selected from N-(3-methoxypropoyl)acrylamide (MPAM) and 4-acryloylmorpholine (MORPH).

The polyacrylamide-based copolymers of the present disclosure also include a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof. In some embodiments, the polyacrylamide-based copolymer includes a hydrophobic functional acrylamide dopant monomer. In some embodiments, the hydrophobic functional acrylamide dopant monomer is N-isopropylacrylamide (NIP) or N-tert-butylacrylamide (TBA). In some embodiments, the hydrophobic functional acrylamide dopant monomer is N-isopropylacrylamide (NIP). In some embodiments, the hydrophobic functional acrylamide dopant monomer is N-tert-butylacrylamide (TBA). In some embodiments, the polyacrylamide-based copolymer includes an aromatic functional acrylamide dopant monomer. In some embodiments, the aromatic functional acrylamide dopant monomer is N-phenylacrylamide (PHE). In some embodiments, the polyacrylamide-based copolymer includes a hydrogen-bonding functional acrylamide dopant monomer. In some embodiments, the hydrogen-bonding functional acrylamide dopant monomer is N-[tris(hydroxymethyl)-methyl]acrylamide (TRI). In some embodiments, the polyacrylamide-based copolymer includes an ionic functional acrylamide dopant monomer. In some embodiments, the ionic functional acrylamide dopant monomer is 2-acrylamido-2-methylpropane sulfonic acid (AMP) or (3-acrylamidopropyl)trimethylammonium chloride (TMA). In some embodiments, the ionic functional acrylamide dopant monomer is 2-acrylamido-2-methylpropane sulfonic acid (AMP). In some embodiments, the ionic functional acrylamide dopant monomer is (3-acrylamidopropyl)trimethylammonium chloride (TMA). In some embodiments, the functional dopant monomer is N,N-diethylacrylamide (DEA).

In some embodiments, the polyacrylamide-based copolymer includes a water-soluble carrier monomer selected from N-(3-methoxypropoyl)acrylamide (MPAM), 4-acryloylmorpholine (MORPH), N,N-dimethylacrylamide (DMA), N-hydroxyethylacrylamide (HEAM), and acrylamide (AM); and a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), and N-phenylacrylamide (PHE).

In some embodiments, the water-soluble carrier monomer is N-(3-methoxypropoyl)acrylamide (MPAM). In some embodiments, the water-soluble carrier monomer is N-(3-methoxypropoyl)acrylamide (MPAM) and the functional dopant monomer is N-phenylacrylamide (PHE).

In some embodiments, the water-soluble carrier monomer is 4-acryloylmorpholine (MORPH). In some embodiments, the water-soluble carrier monomer is 4-acryloylmorpholine (MORPH) and the functional dopant monomer is N-isopropylacrylamide (NIP), or N-phenylacrylamide (PHE).

In some embodiments, the water-soluble earner monomer is N,N-dimethylacrylamide (DMA). In some embodiments, the water-soluble carrier monomer is N,N-dimethylacrylamide (DMA) and the functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof.

In some embodiments, the water-soluble carrier monomer is N-hydroxyethyl acrylamide (HEAM). In some embodiments, the water-soluble carrier monomer is N-hydroxyethyl acrylamide (HEAM) and the functional dopant monomer is selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof.

In some embodiments, the water-soluble carrier monomer is acrylamide (AM). In some embodiments, the water-soluble carrier monomer is acrylamide (AM), and the functional dopant monomer is selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof.

In some embodiments, the polyacrylamide-based copolymer includes N-(3-methoxypropoyl)acrylamide (MPAM) or 4-acryloylmorpholine (MORPH) as the water-soluble carrier monomer, and the functional dopant monomer includes one or more of N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), and N-phenylacrylamide (PHE).

In some embodiments, the polyacrylamide-based copolymer includes N,N-dimethylacrylamide (DMA), N-hydroxyethyl acrylamide (HEAM), or acrylamide (AM) as the water-soluble carrier monomer, and the functional dopant monomer is selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof.

In some embodiments, the amount of functional dopant monomer used in the copolymerization reaction is designed to maximize dopant loading while yielding functional copolymers with lower critical solution temperature (LCST) values above 37° C. In some embodiments, this results in copolymers that remain soluble at all relevant temperatures. In some embodiments, the polyacrylamide-based copolymer includes about 2% to about 30% by weight of a functional dopant monomer, for example, about 5% to about 30%, about 10% to about 30%, about 15% to about 30%, about 20% to about 30%, about 25% to about 30%, about 2% to about 25%, about 5% to about 25%, about 10% to about 25%, about 15% to about 25%, about 20% to about 25%, about 2% to about 20%, about 5% to about 20%, about 10% to about 20%, about 15% to about 20%, about 2% to about 15%, about 5% to about 15%, about 10% to about 15%, about 2% to about 10%, about 5% to about 10%, or about 2% to about 5%, by weight of a functional dopant monomer. In some embodiments, the polyacrylamide-based copolymer includes about 2%, about 5%, about 8%, about 10%, about 12%, about 15%, about 18%, about 20%, about 22%, about 25%, about 28%, or about 30% by weight of a functional dopant monomer. In some embodiments, the polyacrylamide-based copolymer includes 2% to 30% by weight of a functional dopant monomer, for example, 5% to 30%, 10% to 30%, 15% to 30%, 20% to 30%, 25% to 30%, 2% to 25%, 5% to 25%, 10% to 25%, 15% to 25%, 20% to 25%, 2% to 20%, 5% to 20%, 10% to 20%, 15% to 20%, 2% to 15%, 5% to 15%, 10% to 15%, 2% to 10%, 5% to 10%, or 2% to 5%, by weight of a functional dopant monomer. In some embodiments, the polyacrylamide-based copolymer includes 2%, 5%, 8%, 10%, 12%, 15%, 18%, 20%, 22%, 25%, 28%, or 30% by weight of a functional dopant monomer.

In some embodiments, the polyacrylamide-based copolymer includes about 70% to about 98% by weight of a water-soluble carrier monomer, for example, about 75% to about 98%, about 80% to about 98%, about 85% to about 98%, about 90% to about 98%, about 95% to about 98%, about 70% to about 95%, about 75% to about 95%, about 80% to about 95%, about 85% to about 95%, about 90% to about 95%, about 70% to about 90%, about 75% to about 90%, about 80% to about 90%, about 85% to about 90%, about 70% to about 85%, about 75% to about 85%, about 80% to about 85%, about 70% to about 80%, about 75% to about 80%, or about 70% to about 75%, by weight of a water-soluble carrier monomer. In some embodiments, the polyacrylamide-based copolymer includes about 70%, about 72%, about 75%, about 78%, about 80%, about 82%, about 85%, about 88%, about 90%, about 92%, about 95%, or about 98% by weight of a water-soluble carrier monomer. In some embodiments, the polyacrylamide-based copolymer includes 70% to 98% by weight of a water-soluble carrier monomer, for example, 75% to 98%, 80% to 98%, 85% to 98%, 90% to 98%, 95% to 98%, 70% to 95%, 75% to 95%, 80% to 95%, 85% to 95%, 90% to 95%, 70% to 90%, 75% to 90%, 80% to 90%, 85% to 90%, 70% to 85%, 75% to 85%, 80% to 85%, 70% to 80%, 75% to 80%, or 70% to 75%, by weight of a water-soluble carrier monomer. In some embodiments, the polyacrylamide-based copolymer includes 70%, 72%, 75%, 78%, 80%, 82%, 85%, 88%, 90%, 92%, 95%, or 98% by weight of a water-soluble carrier monomer.

In some embodiments, the polyacrylamide-based copolymer includes about 70% to about 98% by weight of a water-soluble carrier monomer and about 2% to about 30% by weight of a functional dopant monomer. For example, the polyacrylamide-based copolymer can contain about 70% to about 98%, about 70% to about 95%, about 70% to about 80%, about 80% to about 95%, about 90% to about 98%, about 70%, about 75%, about 80%, about 85%, about 90%, about 95%, or about 98% by weight of a water-soluble carrier monomer and about 2% to about 30%, about 5% to about 25%, about 5% to about 20%, about 2% to about 5%, about 2% to about 17%, about 5% to about 10%, about 10% to about 15%, about 15% to about 20%, about 20% to about 25%, or about 25% to about 30%, about 2%, about 5%, about 8%, about 10%, about 12%, about 15%, about 18%, about 20%, about 22%, about 25%, about 28%, or about 30% by weight of a functional dopant monomer.

In some embodiments, the polyacrylamide-based copolymer includes 70% to 98% by weight of a water-soluble carrier monomer and 2% to 30% by weight of a functional dopant monomer. For example, the polyacrylamide-based copolymer can contain 70% to 98%, 70% to 95%, 70% to 80%, 80% to 95%, 90% to 98%, 70%, 75%, 80%, 85%, 90%, 95%, or 98% by weight of a water-soluble carrier monomer and 2% to 30%, 5% to 25%, 5% to 20%, 2% to 5%, 2% to 17%, 5% to 10%, 10% to 15%, 15% to 20%, 20% to 25%, or 25% to 30%, 2%, 5%, 8%, 10%, 12%, 15%, 18%, 20%, 22%, 25%, 28%, or 30% by weight of a functional dopant monomer.

In some embodiments, the polyacrylamides-based copolymer includes about 2% to about 30% by weight of the functional dopant monomer NIP. In some embodiments, the polyacrylamides-based copolymer includes about 5% to about 30% by weight of the functional dopant monomer NIP. In some embodiments, the polyacrylamides-based copolymer includes about 10% to about 28% by weight of the functional dopant monomer NIP. In some embodiments, the polyacrylamide-based copolymer includes about 5% to about 26% by weight of the functional dopant monomer NIP. In some embodiments, the polyacrylamides-based copolymer includes about 5% to about 10% by weight of the functional dopant monomer NIP. In some embodiments, the polyacrylamides-based copolymer includes about 10% to about 15% by weight of the functional dopant monomer NIP. In some embodiments, the polyacrylamides-based copolymer includes about 15% to about 20% by weight of the functional dopant monomer NIP. In some embodiments, the polyacrylamides-based copolymer includes about 20% to about 26% by weight of the functional dopant monomer NIP.

In some embodiments, the polyacrylamides-based copolymer includes 2% to 30% by weight of the functional dopant monomer NIP, such as 5% to 30%, 10% to 28%, 5% to 26%, 5% to 10%, 10% to 15%, 15% to 20%, or 20% to 26% by weight of the functional dopant monomer NIP.

In some embodiments, the polyacrylamides-based copolymer includes about 2% to about 30% by weight of the functional dopant monomer PHE. In some embodiments, the polyacrylamides-based copolymer includes about 5% to about 30% by weight of the functional dopant monomer PHE. In some embodiments, the polyacrylamides-based copolymer includes about 10% to about 28% by weight of the functional dopant monomer PHE. In some embodiments, the polyacrylamide-based copolymer includes about 5% to about 26% by weight of the functional dopant monomer PHE. In some embodiments, the polyacrylamides-based copolymer includes about 5% to about 10% by weight of the functional dopant monomer PHE. In some embodiments, the polyacrylamides-based copolymer includes about 10% to about 15% by weight of the functional dopant monomer PHE. In some embodiments, the polyacrylamides-based copolymer includes about 15% to about 20% by weight of the functional dopant monomer PHE. In some embodiments, the polyacrylamides-based copolymer includes about 20% to about 26% by weight of the functional dopant monomer PHE.

In some embodiments, the polyacrylamides-based copolymer includes 2% to 30% by weight of the functional dopant monomer PHE, such as 5% to 30%, 10% to 28%, 5% to 26%, 5% to 10%, 10% to 15%, 15% to 20%, or 20% to 26% by weight of the functional dopant monomer PHE.

In some embodiments, the polyacrylamides-based copolymer includes about 2% to about 30% by weight of the functional dopant monomer DEA. In some embodiments, the polyacrylamides-based copolymer includes about 5% to about 30% by weight of the functional dopant monomer DEA. In some embodiments, the polyacrylamides-based copolymer includes about 10% to about 28% by weight of the functional dopant monomer DEA. In some embodiments, the polyacrylamide-based copolymer includes about 5% to about 26% by weight of the functional dopant monomer DEA. In some embodiments, the polyacrylamides-based copolymer includes about 5% to about 10% by weight of the functional dopant monomer DEA. In some embodiments, the polyacrylamides-based copolymer includes about 10% to about 15% by weight of the functional dopant monomer DEA. In some embodiments, the polyacrylamides-based copolymer includes about 15% to about 20% by weight of the functional dopant monomer DEA. In some embodiments, the polyacrylamides-based copolymer includes about 20% to about 26% by weight of the functional dopant monomer DEA.

In some embodiments, the polyacrylamides-based copolymer includes 2% to 30% by weight of the functional dopant monomer DEA, such as 5% to 30%, 10% to 28%, 5% to 26%, 5% to 10%, 10% to 15%, 15% to 20%, or 20% to 26% by weight of the functional dopant monomer DEA.

In some embodiments, the polyacrylamides-based copolymer includes MORPH as the water-soluble carrier monomer and about 2% to about 30% by weight of a functional dopant monomer selected from NIP, PHE and DEA. In some embodiments, the copolymer includes MORPH and from about 5% to about 30% by weight of a functional dopant monomer selected from NIP, PHE and DEA. In some embodiments, the copolymer includes MORPH and about 10% to about 28% by weight of a functional dopant monomer selected from NIP, PHE and DEA. In some embodiments, the copolymer includes MORPH and about 5% to about 26% by weight of a functional dopant monomer selected from NIP, PHE and DEA. In some embodiments, the copolymer includes MORPH and about 5% to about 10% by weight of a functional dopant monomer selected from NIP, PHE and DEA. In some embodiments, the copolymer includes MORPH and about 10% to about 15% by weight of a functional dopant monomer selected from NIP, PHE and DEA. In some embodiments, the copolymer includes MORPH and about 15% to about 20% by weight of a functional dopant monomer selected from NIP, PHE and DEA. In some embodiments, the copolymer includes MORPH and about 20% to about 25% by weight of a functional dopant monomer selected from NIP, PHE and DEA. In some embodiments, the copolymer includes MORPH and about 25% to about 30% by weight of a functional dopant monomer selected from NIP, PHE and DEA. In some embodiments, the copolymer includes MORPH and about 20% to about 28% by weight of a functional dopant monomer selected from NIP, PHE and DEA. In some embodiments, the copolymer includes MORPH and about 21% by weight of a functional dopant monomer selected from NIP, PHE and DEA. In some embodiments, the copolymer includes MORPH and about 22% by weight of a functional dopant monomer selected from NIP, PHE and DEA. In some embodiments, the copolymer includes MORPH and about 23% by weight of a functional dopant monomer selected from NIP, PHE and DEA. In some embodiments, the copolymer includes MORPH and about 24% by weight of a functional dopant monomer selected from NIP, PHE and DEA. In some embodiments, the copolymer includes MORPH and about 25% by weight of a functional dopant monomer selected from NIP, PHE and DEA.

In some embodiments, the polyacrylamides-based copolymer includes MORPH as the water-soluble carrier monomer and 2% to 30% by weight of a functional dopant monomer selected from NIP, PHE and DEA, such as 5% to 30%, 10% to 28%, 5% to 26%, 5% to 10%, 10% to 15%, 15% to 20%, 20% to 25%, 25% to 30%, or 20% to 28% by weight of a functional dopant monomer selected from NIP, PHE and DEA. In some embodiments, the copolymer includes MORPH and 21% by weight of a functional dopant monomer selected from NIP, PHE and DEA, such as 22, 23%, 24%, or 25% by weight of a functional dopant monomer selected from NIP, PHE and DEA.

In some embodiments, the polyacrylamide-based copolymer includes MPAM as the water-soluble carrier monomer and about 2% to about 16% by weight of a functional dopant monomer selected from NIP, PHE or DEA. In some embodiments, the polyacrylamide-based copolymer includes MPAM as the water-soluble carrier monomer and about 5% to about 15% by weight of a functional dopant monomer selected from NIP, PHE and DEA. In some embodiments, the polyacrylamide-based copolymer includes MPAM as the water-soluble carrier monomer and about 6% to about 10% by weight of a functional dopant monomer selected from NIP, PHE and DEA. In some embodiments, the polyacrylamide-based copolymer includes MPAM as the water-soluble carrier monomer and about 7% by weight of a functional dopant monomer selected from NIP, PHE and DEA. In some embodiments, the polyacrylamide-based copolymer includes MPAM as the water-soluble carrier monomer and about 8% by weight of a functional dopant monomer selected from NIP, PHE and DEA. In some embodiments, the polyacrylamide-based copolymer includes MPAM as the water-soluble carrier monomer and about 9% by weight of a functional dopant monomer selected from NIP, PHE and DEA.

In some embodiments, the polyacrylamide-based copolymer includes MPAM as the water-soluble carrier monomer and 2% to 16% by weight of a functional dopant monomer selected from NIP, PHE and DEA, such as 5% to 15%, or 6% to 10% by weight of a functional dopant monomer selected from NIP, PHE and DEA. In some embodiments, the polyacrylamide-based copolymer includes MPAM as the water-soluble carrier monomer and 7% by weight of a functional dopant monomer selected from NIP, PHE and DEA, such as 8%, or 9% by weight of a functional dopant monomer selected from NIP, PHE and DEA.

In some embodiments, the polyacrylamide-based copolymer further includes TRI, AMP, TMA, or TBA as a functional dopant monomer. In some embodiments, AMP, TMA, or TBA functional dopant monomer is present at about 2% to about 16% by weight of the copolymer. In some embodiments, TRI, AMP, TMA, or TBA functional dopant monomer is present at about 5% to about 15% by weight of the copolymer. In some embodiments, TRI, AMP, TMA, or TBA functional dopant monomer is present at about 6% to about 14% by weight of the copolymer. In some embodiments, TRI, AMP, TMA, or TBA functional dopant monomer is present at about 12% to about 15% by weight of the copolymer. In some embodiments, TRI, AMP, TMA, or TBA functional dopant monomer is present at about 2% to about 5% by weight of the copolymer. In some embodiments, TRI, AMP, TMA, or TBA functional dopant monomer is present at about 5% to about 10% by weight of the copolymer.

In some embodiments, the polyacrylamide-based copolymer further includes AMP, TMA, or TBA as a functional dopant monomer in an amount of 2% to 16%, such as 5% to 15%, 6% to 14%, or 12% to 15% by weight of the copolymer. In some embodiments, TRI, AMP, TMA, or TBA functional dopant monomer is present at 2% to 5% by weight of the copolymer. In some embodiments, TRI, AMP, TMA, or TBA functional dopant monomer is present at 5% to 10% by weight of the copolymer.

In some embodiments, the polyacrylamide-based copolymer includes 70% to 85% by weight of MORPH as the water-soluble carrier monomer and 15% to 30% by weight of NIP as the functional dopant monomer. In some embodiments, the polyacrylamides-based copolymer includes 74% to 80% by weight of MORPH as the water-soluble carrier monomer and 20% to 26% by weight of NIP as the functional dopant monomer. In some embodiments, the polyacrylamide-based copolymer includes 70% by weight of MORPH and 30% by weight of NIP. In some embodiments, the polyacrylamide-based copolymer includes 71% by weight of MORPH and 29% by weight of NIP. In some embodiments, the polyacrylamide-based copolymer includes 72% by weight of MORPH and 28% by weight of NIP. In some embodiments, the polyacrylamide-based copolymer includes 73% by weight of MORPH and 27% by weight of NIP. In some embodiments, the polyacrylamide-based copolymer includes 74% by weight of MORPH and 26% by weight of NIP. In some embodiments, the polyacrylamide-based copolymer includes 75% by weight of MORPH and 25% by weight of NIP. In some embodiments, the polyacrylamide-based copolymer includes 76% by weight of MORPH and 24% by weight of NIP. In some embodiments, the polyacrylamide-based copolymer includes 77% by weight of MORPH and 23% by weight of NIP. In some embodiments, the polyacrylamide-based copolymer includes 78% by weight of MORPH and 22% by weight of NIP. In some embodiments, the polyacrylamide-based copolymer includes 79% by weight of MORPH and 21% by weight of NIP. In some embodiments, the polyacrylamide-based copolymer includes 80% by weight of MORPH and 20% by weight of NIP. In some embodiments, the polyacrylamide-based copolymer includes 81% by weight of MORPH and 19% by weight of NIP. In some embodiments, the polyacrylamide-based copolymer includes 82% by weight of MORPH and 18% by weight of NIP. In some embodiments, the polyacrylamide-based copolymer includes 83% by weight of MORPH and 17% by weight of NIP. In some embodiments, the polyacrylamide-based copolymer includes 84% by weight of MORPH and 16% by weight of NIP. In some embodiments, the polyacrylamide-based copolymer includes 85% by weight of MORPH and 15% by weight of NIP.

In some embodiments, the degree of polymerization (DP) of the polyacrylamide-based copolymer is about 10 to about 500, about 20 to about 200, about 50 to about 100, about 100 to about 200, about 200 to about 300, about 300 to about 400, or about 400 to about 500, or about 50, about 70, about 100, about 120, about 150, about 170, about 200, about 220, about 250, about 270, about 300, about 320, about 350, about 370, about 400, about 420, about 450, about 470, or about 500. In some embodiments, the DP of the copolymer is about 40. In some embodiments, the DP of the copolymer is about 50. In some embodiments, the DP of the copolymer is about 60. In some embodiments, the DP of the copolymer is about 70. In some embodiments, the DP of the copolymer is about 80. In some embodiments, the DP of the copolymer is about 90. In some embodiments, the DP of the copolymer is about 100.

In some embodiments, the degree of polymerization (DP) of the polyacrylamide-based copolymer is 10 to 500, 20 to 200, 50 to 100, 100 to 200, 200 to 300, 300 to 400, or 400 to 500, or 50, 70, 100, 120, 150, 170, 200, 220, 250, 270, 300, 320, 350, 370, 400, 420, 450, 470, or 500. In some embodiments, the DP of the copolymer is 40. In some embodiments, the DP of the copolymer is 50. In some embodiments, the DP of the copolymer is 60. In some embodiments, the DP of the copolymer is 70. In some embodiments, the DP of the copolymer is 80. In some embodiments, the DP of the copolymer is 90. In some embodiments, the DP of the copolymer is 100.

In some embodiments, the molecular weight of the polyacrylamide-based copolymer is about 1,000 g/mol to about 40,000 g/mol, such as about 1,000 g/mol to about 35,000 g/mol, about 1,000 g/mol to about 30,000 g/mol, about 1,000 g/mol to about 25,000 g/mol, about 1,000 g/mol to about 20,000 g/mol, about 1,000 g/mol to about 15,000 g/mol, about 1,000 g/mol to about 10,000 g/mol, about 1,000 g/mol to about 7,000 g/mol, about 1,000 g/mol to about 6,000 g/mol, about 1,000 g/mol to about 5,000 g/mol, about 1,000 g/mol to about 4,000 g/mol, about 1,000 g/mol to about 3,000 g/mol, about 2,000 g/mol to about 10,000 g/mol, about 3,000 g/mol to about 40,000 g/mol, about 3,000 g/mol to about 35,000 g/mol, about 3,000 g/mol to about 30,000 g/mol, about 3,000 g/mol to about 25,000 g/mol, about 3,000 g/mol to about 20,000 g/mol, about 3,000 g/mol to about 15,000 g/mol, about 3,000 g/mol to about 10,000 g/mol, about 3,000 g/mol to about 7,000 g/mol, about 3,000 g/mol to about 6,000 g/mol, about 3,000 g/mol to about 5,000 g/mol, about 3,000 g/mol to about 4,000 g/mol, about 4,000 g/mol to about 40,000 g/mol, about 4,000 g/mol to about 35,000 g/mol, about 4,000 g/mol to about 30,000 g/mol, about 4,000 g/mol to about 25,000 g/mol, about 4,000 g/mol to about 20,000 g/mol, about 4,000 g/mol to about 15,000 g/mol, about 4,000 g/mol to about 10,000 g/mol, about 4,000 g/mol to about 7,000 g/mol, about 4,000 g/mol to about 6,000 g/mol, about 4,000 g/mol to about 5,000 g/mol, about 5,000 g/mol to about 40,000 g/mol, about 5,000 g/mol to about 35,000 g/mol, about 5,000 g/mol to about 30,000 g/mol, about 5,000 g/mol to about 25,000 g/mol, about 5,000 g/mol to about 20,000 g/mol, about 5,000 g/mol to about 15,000 g/mol, about 5,000 g/mol to about 10,000 g/mol, about 5,000 g/mol to about 7,000 g/mol, about 5,000 g/mol to about 6,000 g/mol, about 6,000 g/mol to about 40,000 g/mol, about 6,000 g/mol to about 35,000 g/mol, about 6,000 g/mol to about 30,000 g/mol, about 6,000 g/mol to about 25,000 g/mol, about 6,000 g/mol to about 20,000 g/mol, about 6,000 g/mol to about 15,000 g/mol, about 6,000 g/mol to about 10,000 g/mol, about 6,000 g/mol to about 7,000 g/mol, about 7,000 g/mol to about 40,000 g/mol, about 7,000 g/mol to about 35,000 g/mol, about 7000 g/mol to about 30,000 g/mol, about 7,000 g/mol to about 25,000 g/mol, about 7,000 g/mol to about 20,000 g/mol, about 7,000 g/mol to about 15,000 g/mol, about 7,000 g/mol to about 10,000 g/mol, about 10,000 g/mol to about 40,000 g/mol, about 10,000 g/mol to about 35,000 g/mol, about 10,000 g/mol to about 30,000 g/mol, about 10,000 g/mol to about 25,000 g/mol, about 10,000 g/mol to about 20,000 g/mol, about 10,000 g/mol to about 15,000 g/mol, about 15,000 g/mol to about 40,000 g/mol, about 15,000 g/mol to about 35,000 g/mol, about 15,000 g/mol to about 30,000 g/mol, about 15,000 g/mol to about 25,000 g/mol, about 15,000 g/mol to about 20,000 g/mol, about 20,000 g/mol to about 40,000 g/mol, about 20,000 g/mol to about 35,000 g/mol, about 20,000 g/mol to about 30,000 g/mol, about 20,000 g/mol to about 25,000 g/mol, about 25,000 g/mol to about 40,000 g/mol, about 25,000 g/mol to about 35,000 g/mol, about 25,000 g/mol to about 30,000 g/mol, about 30,000 g/mol to about 40,000 g/mol, about 30,000 g/mol to about 35,000 g/mol, or about 35,000 g/mol to about 40,000 g/mol. In some embodiments, the molecular weight of the copolymer is about 1,000 to about 30,000 g/mol. In some embodiments, the molecular weight of the copolymer is about 10,000 to about 20,000 g/mol. In some embodiments, the molecular weight of the copolymer is about 15,000 to about 20,000 g/mol. In some embodiments, the molecular weight of the copolymer is about 20,000 to about 25,000 g/mol. The molecular weight of the copolymer is about 25,000 to about 30,000 g/mol. In some embodiments, the molecular weight of the copolymer is about 30,000 to about 40,000 g/mol. In some embodiments, the molecular weight of the copolymer is about 2,000 to about 10,000 g/mol. In some embodiments, the molecular weight of the copolymer is about 3,000 to about 7,000 g/mol. In some embodiments, the molecular weight of the copolymer is about 4,000 to about 6,000 g/mol.

In some embodiments, the molecular weight of the polyacrylamide-based copolymer is 1,000 g/mol to 40,000 g/mol, such as 1,000 g/mol to 35,000 g/mol, 1,000 g/mol to 30,000 g/mol, 1,000 g/mol to 25,000 g/mol, 1,000 g/mol to 20,000 g/mol, 1,000 g/mol to 15,000 g/mol, 1,000 g/mol to 10,000 g/mol, 1,000 g/mol to 7,000 g/mol, 1,000 g/mol to 6,000 g/mol, 1,000 g/mol to 5,000 g/mol, 1,000 g/mol to 4,000 g/mol, 1,000 g/mol to 3,000 g/mol, 2,000 g/mol to 10,000 g/mol, 3,000 g/mol to 40,000 g/mol, 3,000 g/mol to 35,000 g/mol, 3,000 g/mol to 30,000 g/mol, 3,000 g/mol to 25,000 g/mol, 3,000 g/mol to 20,000 g/mol, 3,000 g/mol to 15,000 g/mol, 3,000 g/mol to 10,000 g/mol, 3,000 g/mol to 7,000 g/mol, 3,000 g/mol to 6,000 g/mol, 3,000 g/mol to 5,000 g/mol, 3,000 g/mol to 4,000 g/mol, 4,000 g/mol to 40,000 g/mol, 4,000 g/mol to 35,000 g/mol, 4,000 g/mol to 30,000 g/mol, 4,000 g/mol to 25,000 g/mol, 4,000 g/mol to 20,000 g/mol, 4,000 g/mol to 15,000 g/mol, 4,000 g/mol to 10,000 g/mol, 4,000 g/mol to 7,000 g/mol, 4,000 g/mol to 6,000 g/mol, 4,000 g/mol to 5,000 g/mol, 5,000 g/mol to 40,000 g/mol, 5,000 g/mol to 35,000 g/mol, 5,000 g/mol to 30,000 g/mol, 5,000 g/mol to 25,000 g/mol, 5,000 g/mol to 20,000 g/mol, 5,000 g/mol to 15,000 g/mol, 5,000 g/mol to 10,000 g/mol, 5,000 g/mol to 7,000 g/mol, 5,000 g/mol to 6,000 g/mol, 6,000 g/mol to 40,000 g/mol, 6,000 g/mol to 35,000 g/mol, 6,000 g/mol to 30,000 g/mol, 6,000 g/mol to 25,000 g/mol, 6,000 g/mol to 20,000 g/mol, 6,000 g/mol to 15,000 g/mol, 6,000 g/mol to 10,000 g/mol, 6,000 g/mol to 7,000 g/mol, 7,000 g/mol to 40,000 g/mol, 7,000 g/mol to, 35,000 g/mol, 7000 g/mol to 30,000 g/mol, 7,000 g/mol to 25,000 g/mol, 7,000 g/mol to 20,000 g/mol, 7,000 g/mol to 15,000 g/mol, 7,000 g/mol to 10,000 g/mol, 10,000 g/mol to 40,000 g/mol, 10,000 g/mol to 35,000 g/mol, 10,000 g/mol to 30,000 g/mol, 10,000 g/mol to 25,000 g/mol, 10,000 g/mol to 20,000 g/mol, 10,000 g/mol to 15,000 g/mol, 15,000 g/mol to 40,000 g/mol, 15,000 g/mol to 35,000 g/mol, 15,000 g/mol to 30,000 g/mol, 15,000 g/mol to 25,000 g/mol, 15,000 g/mol to 20,000 g/mol, 20,000 g/mol to 40,000 g/mol, 20,000 g/mol to 35,000 g/mol, 20,000 g/mol to 30,000 g/mol, 20,000 g/mol to 25,000 g/mol, 25,000 g/mol to 40,000 g/mol, 25,000 g/mol to 35,000 g/mol, 25,000 g/mol to 30,000 g/mol, 30,000 g/mol to 40,000 g/mol, 30,000 g/mol to 35,000 g/mol, or 35,000 g/mol to 40,000 g/mol. In some embodiments, the molecular weight of the copolymer is 1,000 to 30,000 g/mol. In some embodiments, the molecular weight of the copolymer is 10,000 to 20,000 g/mol. In some embodiments, the molecular weight of the copolymer is 15,000 to 20,000 g/mol. In some embodiments, the molecular weight of the copolymer is 20,000 to 25,000 g/mol. The molecular weight of the copolymer is 25,000 to 30,000 g/mol. In some embodiments, the molecular weight of the copolymer is 30,000 to 40,000 g/mol. In some embodiments, the molecular weight of the copolymer is 2,000 to 10,000 g/mol. In some embodiments, the molecular weight of the copolymer is 3,000 to 7,000 g/mol. In some embodiments, the molecular weight of the copolymer is 4,000 to 6,000 g/mol.

In some embodiments, the polyacrylamide-based copolymer contains a water-soluble carrier monomer comprising an acrylamide reactive moiety, and a functional dopant monomer (as described herein). In some embodiments, the polyacrylamide-based copolymer includes about 70% to about 98% of a water-soluble carrier monomer with an acrylamide reactive moiety and about 2% to about 30% of a functional dopant monomer. In some embodiments, the number-averaged molecular weight (Mn) of the copolymer is about 1,000 g/mol to about 30,000 g/mol. In some embodiments, the degree of polymerization is about 10 to about 250. In some embodiments, the polyacrylamide-based copolymer includes 70% to 98% of a water-soluble carrier monomer with an acrylamide reactive moiety and 2% to 30% of a functional dopant monomer. In some embodiments, the number-averaged molecular weight (Mn) of the copolymer is 1,000 g/mol to 30,000 g/mol. In some embodiments, the degree of polymerization is 10 to 250. In some embodiments, the water-soluble carrier monomer is non-ionic. In some embodiments, the copolymer is amphiphilic.

In some embodiments, the polyacrylamide-based copolymer includes about 70% to about 95% by weight of the water-soluble carrier monomer MORPH and about 5% to about 30% by weight of the functional dopant monomer NIP, wherein the number-averaged molecular weight (Mn) of the copolymer is about 1,000 g/mol to about 10,000 g/mol and the degree of polymerization is about 10 to about 100. In some embodiments, the polyacrylamide-based copolymer includes about 74% to about 80% by weight of the water-soluble carrier monomer MORPH and about 20% to about 26% by weight of the functional dopant monomer NIP, wherein the number-averaged molecular weight (Mn) of the copolymer is about 1,000 g/mol to about 5,000 g/mol and the degree of polymerization is about 10 to about 50. In some embodiments, the polyacrylamide-based copolymer includes about 77% by weight of the water-soluble carrier monomer MORPH and about 23% by weight of the functional dopant monomer NIP, wherein the number-averaged molecular weight (Mn) of the copolymer is about 3,200 g/mol and the degree of polymerization is about 26.

In some embodiments, the polyacrylamide-based copolymer includes 70% to 95% by weight of the water-soluble carrier monomer MORPH and 5% to 30% by weight of the functional dopant monomer NIP, wherein the number-averaged molecular weight (Mn) of the copolymer is 1,000 g/mol to 10,000 g/mol and the degree of polymerization is 10 to 100. In some embodiments, the polyacrylamide-based copolymer includes 74% to 80% by weight of the water-soluble carrier monomer MORPH and 20% to 26% by weight of the functional dopant monomer NIP, wherein the number-averaged molecular weight (Mn) of the copolymer is 1,000 g/mol to 5,000 g/mol and the degree of polymerization is 10 to 50. In some embodiments, the polyacrylamide-based copolymer includes 77% by weight of the water-soluble carrier monomer MORPH and 23% by weight of the functional dopant monomer NIP, wherein the number-averaged molecular weight (Mn) of the copolymer is 3,200 g/mol and the degree of polymerization is 26.

In some embodiments, the particles include about 0.01 wt % to about 25 wt % of the polyacrylamide-based copolymer, for example, about 0.01 wt % to about 20 wt %, about 0.01 wt % to about 10 wt %, about 0.01 wt % to about 5 wt %, about 0.1 wt % to about 5 wt %, about 0.2 wt % to about 5 wt %, about 0.3 wt % to about 5 wt %, about 0.4 wt % to about 5 wt %, about 0.5 wt % to about 5 wt %, about 0.6 wt % to about 5 wt %, about 0.7 wt % to about 5 wt %, about 0.8 wt % to about 5 wt %, about 0.9 wt % to about 5 wt %, about 1 wt % to about 5 wt %, about 0.1 wt % to about 4 wt %, about 0.1 wt % to about 3 wt %, about 0.1 wt % to about 2 wt %, or about 0.1 wt % to about 1 wt % of the polyacrylamide-based copolymer. In some embodiments, the particles includes about 5 wt % of the polyacrylamide-based copolymer.

In some embodiments, the particles include 0.1 wt % to 25 wt % of the polyacrylamide-based copolymer, for example, 0.1 wt % to 20 wt %, 0.1 wt % to 10 wt %, 0.1 wt % to 5 wt %, 0.2 wt % to 5 wt %, 0.3 wt % to 5 wt %, 0.4 wt % to 5 wt %, 0.5 wt % to 5 wt %, 0.6 wt % to 5 wt %, 0.7 wt % to 5 wt %, 0.8 wt % to 5 wt %, 0.9 wt % to 5 wt %, 1 wt % to 5 wt %, 0.1 wt % to 4 wt %, 0.1 wt % to 3 wt %, 0.1 wt % to 2 wt %, or 0.1 wt % to 1 wt % of the polyacrylamide-based copolymer. In some embodiments, the particles include 5 wt % to 25 wt % of the polyacrylamide-based copolymer, for example, about 5 wt % to about 10 wt %. In some embodiments, the particles include 5 wt % of the polyacrylamide-based copolymer.

### 4.1.3. Optional Particle Components

The pharmaceutical composition of this disclosure can further include one or more additional components, such as excipients. In some embodiments, such additional components, or excipients are formulated into the particles of the composition.

The term “excipient” refers to a natural or synthetic substance formulated alongside the active biopharmaceutical agent of a composition, included for the purpose of stabilization, bulking, and/or to confer a therapeutic enhancement on the biopharmaceutical agent in the final dosage form, such as facilitating drug absorption, reducing viscosity, enhancing or reducing aqueous or non-aqueous solubility, adjusting tonicity, mitigating injection site discomfort, depressing the freezing point, or enhancing stability. Excipients can also be useful in the manufacturing process, to aid in the handling of the biopharmaceutical agent concerned such as by facilitating powder flowability or non-stick properties, in addition to aiding stability such as prevention of denaturation or aggregation over the expected shelf life.

The term “pharmaceutically acceptable” ingredient, excipient or component is one that is suitable for use with humans and/or animals without undue adverse side effects (such as toxicity, irritation, and allergic response) commensurate with a reasonable benefit/risk ratio.

Additional components, or excipients, of interest include a variety of materials for modifying, maintaining or preserving, for example, the pH, osmolarity, viscosity, clarity, color, tonicity, odor, sterility, stability, rate of dissolution or release, adsorption or penetration of the composition. Suitable formulation materials include, but are not limited to, amino acids; antimicrobials; antioxidants; buffers (e.g., a phosphate buffer); chelating agents; complexing agents; saccharides (monosaccharides, disaccharides, polysaccharides, and other carbohydrates (e.g., mannitol, sorbitol, sucrose, trehalose, lactose, melibiose, cyclodextrins, stachyose, lactosucrose, melezidose, raffinose, inulin, chitosan, alginate, hyaluronan cellulosics, dextrans, alginates, etc.)); synthetic polymers (e.g., polyoxamers, polyvinylalcohol, polyvinylpyrrolidone, pluronics, etc.); emulsifying agents (e.g., polysorbates); salt-forming counterions; preservatives; solvents; sugar alcohols; suspending agents; surfactants or wetting agents; stability enhancing agents; tonicity enhancing agents; delivery vehicles; diluents; other excipients and/or pharmaceutical adjuvants. In accordance with appropriate industry standards, preservatives may also be added. The composition may be formulated as a lyophilizate using appropriate excipient solutions as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th} Ed. (2000), Mack Publishing Company, Easton, PA.

In some embodiments, the composition (e.g., particles) further comprises one or more of stabilizing agent, preservative, filler, bulking agent, sugar, polysaccharide, or viscosity modifier.

In certain embodiments, the particles include a stabilizing agent. In some embodiments, the stabilizing agent is selected from surfactants, poloxamers, povidones, polyvinylpyrrolidone (PVP) polymer, polyvinyl alcohol (PVA) polymer, polysaccharides (e.g., dextrans, alginates), cellulosics (e.g., hydroxypropyl methyl cellulose (HPMC), methyl cellulose (MC)), amphoteric compounds, sugars, salts, and combinations thereof.

### 4.2. Injectable Compositions of Particles

Also provided are injectable pharmaceutical compositions containing a suspension of the particles described in the present disclosure in a liquid carrier. Described herein are exemplary components of the particles, and methods of making particles which are utilized in the pharmaceutical compositions suitable for injection. Also described are the components of the injectable pharmaceutical compositions, other than the particles, such as the liquid carrier. It is understood that the components of the compositions can be described in terms of their content in the solid particles, and/or in reference to their content in the final injectable pharmaceutical compositions, which includes both an amount of the solid particles and an amount of the liquid carrier.

In some embodiments, the composition that contains suspended particles of the present disclosure has a copolymer concentration of about 0.01% to about 10% by weight of the composition, such as about 0.01% to about 5% by weight of the composition. In some embodiments, the copolymer concentration is about 0.1% to about 5% by weight of the composition.

In some embodiments, the composition that contains suspended particles of the present disclosure has about 20 wt % or more, for example, about 25 wt % or more, about 30 wt % or more, about 40 wt % or more, about 50 wt % or more, about 60 wt % or more, about 65 wt % or more, about 70 wt % or more, about 75 wt % or more, about 80 wt % or more, about 85 wt % or more, about 90 wt % or more, about 95 wt % or more of the biopharmaceutical agent. In some embodiments, the composition includes about 20 wt % to 65 wt % of the biopharmaceutical agent, for example, about 40 wt % to 65 wt %, or 35 wt % to 50 wt % of the biopharmaceutical agent.

In some embodiments, the composition that contains suspended particles of the present disclosure has 20 wt % or more, for example, 25 wt % or more, 30 wt % or more, 40 wt % or more, 50 wt % or more, 60 wt % or more, 65 wt % or more, 70 wt % or more, 75 wt % or more, 80 wt % or more, 85 wt % or more, 90 wt % or more, 95 wt % or more of the biopharmaceutical agent. In some embodiments, the composition includes 20 wt % to 80 wt % of the biopharmaceutical agent, for example, 35 wt % to 80 wt %, such as 40 wt % to 65 wt %, or 35 wt % to 50 wt % of the biopharmaceutical agent.

In some embodiments, the biopharmaceutical agent is an antibody.

In some embodiments, the suspended particles contain an antibody. In some embodiments, the antibody is a monoclonal antibody or fragment thereof. In some embodiments, the composition has an antibody concentration of about 20 wt % or more, for example, about 25 wt % or more, about 30 wt % or more, about 40 wt % or more, about 50 wt % or more, about 60 wt % or more, about 65 wt % or more, about 70 wt % or more, about 75 wt % or more, about 80 wt % or more, about 85 wt % or more, about 90 wt % or more, about 95 wt % or more of the antibody. In some embodiments, the composition includes about 20 wt % to 65 wt % of the antibody, for example, about 40 wt % to 65 wt %, or 35 wt % to 50 wt % of the antibody.

In some embodiments, the composition has 20 wt % or more, for example, 25 wt % or more, 30 wt % or more, 40 wt % or more, 50 wt % or more, 60 wt % or more, 65 wt % or more, 70 wt % or more, 75 wt % or more, 80 wt % or more, 85 wt % or more, 90 wt % or more, 95 wt % or more of an antibody or fragment thereof. In some embodiments, the composition includes 20 wt % to 80 wt % of the biopharmaceutical agent, for example, 35 wt % to 80 wt %, 40 wt % to 65 wt %, or 35 wt % to 50 wt % of the antibody.

In some embodiments, the composition that contains suspended particles of the present disclosure has no more than 20 wt % of the biopharmaceutical agent, for example, no more than 15 wt %, no more than 10 wt %, no more than 9 wt %, no more than 8 wt %, no more than 7 wt %, no more than 6 wt %, no more than 5 wt %, no more than 4 wt %, no more than 3 wt %, no more than 2 wt %, or no more than 1 wt % of the biopharmaceutical agent. In some embodiments, the composition includes 0.1 wt % to 20 wt % of the biopharmaceutical agent, for example 0.1 wt % to about 10 wt %, or 0.1 wt % to about 5 wt %. In some embodiments, the composition includes 1 wt % to 20 wt % of the biopharmaceutical agent, for example 1 wt % to 10 wt %.

In some embodiments, the biopharmaceutical agent is a peptide or peptide analogue.

In some embodiments, the suspended particles contain insulin or an analog thereof. In some embodiments, the composition includes about 1 wt % to about 20 wt %, about 1 wt % to about 15 wt %, about 1 wt % to about 10 wt %, about 1 wt % to about 9 wt %, about 1 wt % to about 8 wt % of the insulin or the analog thereof. In some embodiments, the composition includes about 7 wt %, about 8 wt %, about 9 wt %, or about 10 wt % of the insulin or the analog thereof. In some embodiments, the composition includes 1 wt % to 20 wt %, 1 wt % to 15 wt %, 1 wt % to 10 wt %, 1 wt % to 9 wt %, or 1 wt % to 8 wt % of the insulin or the analog thereof. In some embodiments, the composition includes 7 wt %, 8 wt %, 9 wt %, or 10 wt % of the insulin or the analog thereof.

In some embodiments, the concentration of the insulin or the insulin analog in the composition is about U50 (that is, 50 U/mL) to about U1000, about U50 to about U500, about U50 to about U200, about U50 to about U100, about U100 to about U500, or about U100 to about U200. In some embodiments, the concentration of the insulin or the insulin analog in the composition is about U50, about U100, about U200, about U500, or about U1000. In some embodiments, the composition includes about 1.7 mg/mL to about 17.5 mg/mL, about 1.7 mg/mL to about 7 mg/mL, about 1.7 mg/mL to about 3.5 mg/mL, about 3.5 wt % to about 17.5 mg/mL, or about 3.5 mg/mL to about 7 mg/mL of the insulin or the analog thereof. In some embodiments, the composition includes about 1.7 mg/mL, about 3.5 mg/mL, about 7 mg/mL, or about 17.5 mg/mL of the insulin or the analog thereof. In some embodiments, the concentration of the insulin or the insulin analog in the composition is U50 (that is, 50 U/mL) to U500, U50 to U200, U50 to U100, U100 to U500, or U100 to U200. In some embodiments, the concentration of the insulin or the insulin analog in the composition is U50, U100, U200, or U500. In some embodiments, the composition includes 1.7 mg/mL to 17.5 mg/mL, 1.7 mg/mL to 7 mg/mL, 1.7 mg/mL to 3.5 mg/mL, 3.5 mg/mL to 17.5 mg/mL, or 3.5 mg/mL to 7 mg/mL of the insulin or the analog thereof. In some embodiments, the composition includes 1.7 mg/mL, 3.5 mg/mL, 7 mg/mL, or 17.5 mg/mL of the insulin or the analog thereof.

In some embodiments, the suspended particles comprise a co-formulation of particles comprising biopharmaceutical agents. In some embodiments the suspended particles comprise a co-formulation of a first batch of particles comprising a biopharmaceutical agent that is insulin or an insulin analog, and a second batch of particles biopharmaceutical agent that is a peptide or a peptide analog. In some embodiments, the suspended particles comprise a co-formulation of particles comprising insulin or an insulin analog, and particles comprising pramlintide. In some embodiments, the composition includes 1.0 mg/mL to 35 mg/mL, 1.0 mg/mL to 17.5 mL, 1.0 mg/mL to 7.0 mg/mL, 1.5 mg/mL to 5.0 mg/mL, 2.0 mg/mL to 5.0 mg/mL, 2.5 mg/mL to 4.5 mg/mL, or 3.0 mg/mL to 4.0 mg/mL of the insulin or the analog thereof; and 0.2 mg/mL to 2.0 mg/mL, 0.2 mg/mL to 1.5 mg/mL, 0.2 mg/mL to 1.0 mg/mL, 0.2 mg/mL to 1.2 mg/mL, 0.5 mg/mL to 1.0 mg/mL, or 0.6 mg/mL to 0.9 mg/mL of the pramlintide. In some embodiments, the composition includes 1.5 mg/mL, 3.5 mg/mL, 5 mg/mL, 7 mg/mL, 17.5 mg/mL or 35 mg/mL of the insulin or the analog thereof; and 0.2, 0.5, 0.6, 0.8, 1.0, or 1.2 mg/mL of pramlintide.

In some embodiments, the composition includes a 1:500 to 1:1 weight to weight ratio of the polyacrylamide-based copolymer to the biopharmaceutical agent.

In some embodiments, the composition includes a 1:10 to 1:2 weight to weight ratio (e.g., a 1:5 weight to weight ratio) of the polyacrylamide-based copolymer to the biopharmaceutical agent.

In some embodiments, the composition includes a 1:25 to 1:15 weight to weight ratio (e.g., a 1:20 weight to weight ratio) of the polyacrylamide-based copolymer to the biopharmaceutical agent.

### 4.2.1. Liquid Carrier

As summarized above, the injectable compositions described herein include particles suspended in a liquid carrier. In some embodiments, the liquid carrier is non-aqueous. In some embodiments, the liquid carrier is an organic liquid. In some embodiments, the liquid carrier includes an organic liquid and an aqueous solution. In some embodiments, the liquid carrier is referred to as a pharmaceutically acceptable liquid carrier.

The term “pharmaceutically acceptable liquid carrier” means a pharmaceutically acceptable solvent, liquid suspending agent or liquid vehicle for delivering a biopharmaceutical agent of the present disclosure to the animal or human.

In some embodiments the organic liquid includes an organic solvent, an oil, or a combination thereof. In some embodiments, the liquid carrier is an organic solvent. It is understood that in the present disclosure an organic “solvent” does not necessarily dissolve the suspended particles of biopharmaceutical agent and polyacrylamide-based copolymer, but rather is a term of art that refers to organic liquids of interest which can be utilized in the present injectable pharmaceutical compositions.

In some embodiments, the liquid carrier includes an organic solvent, and an aqueous solution, such as water or a buffer. In some embodiments, the liquid carrier is a mixture of an organic liquid miscible with water that does not dissolve the particles and/or the biopharmaceutical agent. In some embodiments, an amount of water is included in the liquid carrier as a viscosity reducer. In some embodiments, the liquid carrier includes 10% by weight or less of the aqueous solution (e.g., water) in a miscible organic solvent such that the resulting liquid carrier mixture does not dissolve the particles and/or the biopharmaceutical agent.

In some embodiments, the organic liquid includes an organic solvent. An organic solvent refers to a low MW carbon-based substance capable of dissolving or dispersing one or more other substances. In the pharmaceutical compositions of this disclosure, the organic solvent can be a liquid capable of dispersing or suspending, but not dissolving, the suspended particles (e.g., as described herein). It is understood that one or more selected excipients may be dissolved in the organic liquid of the compositions.

In some embodiments, the organic liquid is selected from triacylglyceride, diacylglyceride, monoacylglyceride, an acetamide, alkyl alcohol, aryl alcohol, aralkyl alcohol, fatty acid or a fatty acid ester, oil, alkane, perfluoroalkane, propylene glycol monoester, propylene glycol diester, butylene glycol monoester, butylene glycol diester, polyethylene glycol diester, and combinations thereof.

In some embodiments, the organic liquid is a monoacylglyceride. A monoacylglyceride is derived from glycerol and one fatty acid connected to the glycerol via an ester linkage. A monoacylglyceride can be a 1-monoacylglycerol or a 2-monoacylglycerol. In some embodiments, the monoacylglyceride includes a fatty acid chain (saturated or unsaturated, branched or linear) having 4-28 carbon atoms, such as 8-20 carbon atoms. In some embodiments, the fatty acid chain has 8-12 carbon atoms, or 8-10 carbon atoms. In some embodiments, the fatty acid ester groups of the propylene glycol diester are selected from oleic acid, myristic acid, caprylic acid, and capric acid. In some embodiments, the fatty acid is a saturated fatty acid. In some embodiments, the fatty acid is an unsaturated fatty acid, such as linoleic acid, myristoleic acid, palmitoleic acid, or oleic acid. In some embodiments, the fatty acid is selected from hexanoic acid, caprylic acid, capric acid, lauric acid, myristic acid, palmitic acid, stearic acid, linoleic acid, myristoleic acid, palmitoleic acid, and oleic acid.

In some embodiments, the organic liquid is a diacylglyceride. A diacylglyceride is derived from glycerol and two fatty acids connected to the glycerol via ester linkages. A diacylglyceride can be a 1,2-diacylglycerol or a 1,3-diacylglycerol. In some embodiments, the diacylglyceride includes two fatty acid chains (saturated or unsaturated, branched or linear) each independently having 4-28 carbon atoms, such as 8-20 carbon atoms. In some embodiments, the fatty acid chains each have 8-12 carbon atoms or 8-10 carbon atoms. In some embodiments, the fatty acid ester groups of the propylene glycol diester are selected from oleic acid, myristic acid, caprylic acid, capric acid, or any combination thereof. In some embodiments, the fatty acid is a saturated fatty acid. In some embodiments, the fatty acid is an unsaturated fatty acid, such as linoleic acid, myristoleic acid, palmitoleic acid, or oleic acid. In some embodiments, the fatty acids include both a saturated fatty acid and an unsaturated fatty acid. In some embodiments, the fatty acid is selected from hexanoic acid, caprylic acid, capric acid, lauric acid, myristic acid, palmitic acid, stearic acid, linoleic acid, myristoleic acid, palmitoleic acid, and oleic acid.

In some embodiments, the organic liquid includes a triacylglyceride. In some embodiments, the triacylglyceride is triacetin.

In some embodiments, the organic liquid includes a triacylglyceride having three fatty acid chains. In some embodiments, the organic liquid includes a triacylglyceride having two fatty acid chains. In some embodiments, the organic liquid includes a triacylglyceride that has one fatty acid chain. In some embodiments, the fatty acid chains are hydrocarbon chains (saturated or unsaturated, branched or linear) each independently including from 4 to 28 carbon atoms, such as from 8 to 20, 8 to 12, or 8 to 10 carbon atoms. In some embodiments, the fatty acid chain is selected from caprylic acid and capric acid. In some embodiments, the fatty acid is a saturated fatty acid. In some embodiments, the fatty acid is an unsaturated fatty acid, such as linoleic acid, myristoleic acid, palmitoleic acid, or oleic acid. In some embodiments, the fatty acids include both a saturated fatty acid and an unsaturated fatty acid. In some embodiments, the fatty acid is selected from hexanoic acid, caprylic acid, capric acid, lauric acid, myristic acid, palmitic acid, stearic acid, linoleic acid, myristoleic acid, palmitoleic acid, and oleic acid. Triacylglyceride liquids of interest include, but are not limited to, Miglyol® 829.

In some embodiments, the organic liquid includes an acetamide. An acetamide liquid of interest includes, but is not limited to, acetamide (CH₃CONH₂), N-alkyl-acetamide and N,N-dialkyl-acetamide. In some embodiments, the acetamide is selected from acetamide (CH₃CONH₂), N-methylacetamide, N-ethylacetamide, N,N-diethylacetamide and N,N-dimethylacetamide. In some embodiments, the acetamide is N,N-dimethylacetamide (DMAc).

In some embodiments, the organic liquid includes an alkyl alcohol, aryl alcohol, an aralkyl alcohol, or a combination thereof. In some embodiments, the organic liquid includes octanol. In some embodiments, the organic liquid includes isopropyl alcohol. In some embodiments, the organic liquid includes ethyl alcohol. In some embodiments, the organic liquid includes benzyl alcohol.

In some embodiments, the organic liquid includes an alkyl benzoate, an aryl benzoate, an aralkyl benzoate, or any combination thereof. In some embodiments, the organic liquid includes benzyl benzoate.

In some embodiments, the organic liquid includes a polar aprotic solvent. In some embodiments, the polar aprotic solvent includes dimethyl sulfoxide (DMSO), N-methyl-2-pyrrolidone (NMP), or mixtures thereof.

In some embodiments, the organic liquid includes a propylene glycol (PG) solvent.

In some embodiments, the organic liquid includes propylene glycol diester. In some embodiments, the propylene glycol diester is a propylene glycol diester of fatty acid(s). In some embodiments, the fatty acid ester groups of the propylene glycol diester include a hydrocarbon chain (saturated or unsaturated, branched or linear) including from 4 to 28 carbon atoms, such as from 8 to 20 carbon atoms. In some embodiments, the fatty acid ester groups of the propylene glycol diester are selected from oleic acid, myristic acid, caprylic acid, capric acid, or any combination thereof.

In some embodiments, the propylene glycol diester includes propylene glycol diesters of caprylic acid, propylene glycol diesters of capric acid, or a combination thereof. In some embodiments, the propylene glycol diester includes diester of caprylic acid and/or capric acid. In some embodiments, the propylene glycol diester is propylene glycol dicaprylate. In some embodiments, the propylene glycol diester is propylene glycol dicaprate. Propylene glycol diesters of interest include, but are not limited to, Miglyol® 840.

In some embodiments, the organic liquid is a propylene glycol monoester. In some embodiments, the propylene glycol monoester includes one fatty acid ester having a chain (saturated or unsaturated, branched or linear) of 4-28 carbon atoms, such as 8-20 carbon atoms. In some embodiments, the fatty acid chain has 8-10 carbon atoms. In some embodiments, the fatty acid ester groups of the propylene glycol monoester are selected from oleic acid, myristic acid, caprylic acid, and capric acid. In some embodiments, the fatty acid is a saturated fatty acid. In some embodiments, the fatty acid is an unsaturated fatty acid, such as linoleic acid, myristoleic acid, palmitoleic acid, or oleic acid. In some embodiments, the fatty acid is selected from hexanoic acid, caprylic acid, capric acid, lauric acid, myristic acid, palmitic acid, stearic acid, linoleic acid, myristoleic acid, palmitoleic acid, and oleic acid.

In some embodiments, the organic liquid includes a butylene glycol diester. In some embodiments, the butylene glycol diester is a butylene glycol diester of fatty acid(s). In some embodiments, the fatty acid ester groups of the butylene glycol diester include a hydrocarbon chain (saturated or unsaturated, branched or linear) including from 4 to 28 carbon atoms, such as from 8 to 20 carbon atoms, 8 to 12, or 8 to 10. In some embodiments, the fatty acid ester groups of the butylene glycol diester are selected from oleic acid, myristic acid, caprylic acid, capric acid, or any combination thereof. In some embodiments, the butylene glycol solvent includes propylene glycol diester of caprylic acid, propylene glycol diester of capric acid, or a combination thereof. In some embodiments, the butylene glycol diester includes diester of caprylic acid and capric acid (e.g., butylene glycolester of caprylic/capric acid). In some embodiments, the fatty acid is a saturated fatty acid. In some embodiments, the fatty acid is an unsaturated fatty acid, such as linoleic acid, myristoleic acid, palmitoleic acid, or oleic acid. In some embodiments, the fatty acids include both a saturated fatty acid and an unsaturated fatty acid. In some embodiments, the fatty acid is selected from hexanoic acid, caprylic acid, capric acid, lauric acid, myristic acid, palmitic acid, stearic acid, linoleic acid, myristoleic acid, palmitoleic acid, and oleic acid. Butylene glycol diesters of interest include, but are not limited to, Miglyol® 8810.

In some embodiments, the organic liquid is a butylene glycol monoester. In some embodiments, the butylene glycol monoester includes one fatty acid ester having a chain (saturated or unsaturated, branched or linear) of 4-28 carbon atoms, such as 8-20 carbon atoms. In some embodiments, the fatty acid chain has 8-10 carbon atoms. In some embodiments, the fatty acid ester groups of the butylene glycol monoester are selected from oleic acid, myristic acid, caprylic acid, and capric acid. In some embodiments, the fatty acid is a saturated fatty acid. In some embodiments, the fatty acid is an unsaturated fatty acid, such as linoleic acid, myristoleic acid, palmitoleic acid, or oleic acid. In some embodiments, the fatty acid is selected from hexanoic acid, caprylic acid, capric acid, lauric acid, myristic acid, palmitic acid, stearic acid, linoleic acid, myristoleic acid, palmitoleic acid, and oleic acid.

In some embodiments, the organic liquid includes a polyethylene glycol (PEG) solvent. In some embodiments, the PEG solvent is PEG 200, PEG 400, or a combination thereof.

In some embodiments, the organic liquid is a polyethylene glycol diester. In some embodiments, the polyethylene glycol portion of the polyethylene glycol diester has an average MW of 500 or less, such as 400 or less, 200-400, or 300-400. In some embodiments, the polyethylene glycol diester has two fatty acid ester groups linked to the linear polyethylene glycol. In some embodiments, the polyethylene glycol diester includes fatty acid esters having a chain (saturated or unsaturated, branched or linear) of 4-28 carbon atoms, such as 8-20 carbon atoms. In some embodiments, the fatty acid chain has 8-10 carbon atoms. In some embodiments, the fatty acid ester groups of the polyethylene glycol diester are selected from oleic acid, myristic acid, caprylic acid, and capric acid. In some embodiments, the fatty acid is a saturated fatty acid. In some embodiments, the fatty acid is an unsaturated fatty acid, such as linoleic acid, myristoleic acid, palmitoleic acid, or oleic acid. In some embodiments, the fatty acids include both a saturated fatty acid and an unsaturated fatty acid. In some embodiments, the fatty acid is selected from hexanoic acid, caprylic acid, capric acid, lauric acid, myristic acid, palmitic acid, stearic acid, linoleic acid, myristoleic acid, palmitoleic acid, and oleic acid.

In some embodiments, the organic liquid includes a fatty acid or a fatty acid ester. In some embodiments, the fatty acid or fatty acid ester includes a hydrocarbon chain (saturated or unsaturated, branched or linear) including from 4 to 28 carbon atoms, such as from 8 to 20 carbon atoms, 8 to 16, 8 to 12, or 8 to 10 carbon atoms. In some embodiments, the fatty acid is a saturated fatty acid. In some embodiments, the fatty acid is an unsaturated fatty acid, such as linoleic acid, myristoleic acid, palmitoleic acid, or oleic acid. In some embodiments, the fatty acid is selected from hexanoic acid, caprylic acid, capric acid, lauric acid, myristic acid, palmitic acid, stearic acid, linoleic acid, myristoleic acid, palmitoleic acid, and oleic acid. In some embodiments, the fatty acid is oleic acid, myristic acid, caprylic acid, capric acid, or any combination thereof. In some embodiments, the fatty acid ester is derived from oleic acid, myristic acid, caprylic acid, or capric acid. In some embodiments, the fatty acid ester is derived from hexanoic acid, caprylic acid, capric acid, lauric acid, myristic acid, palmitic acid, stearic acid, linoleic acid, myristoleic acid, palmitoleic acid, or oleic acid. In some embodiments, the fatty acid ester is an (C₁-C₆)-alkyl or substituted (C₁-C₆)-alkyl ester of a fatty acid (e.g., as described herein). In some embodiments, the fatty acid ester includes ethyl oleate, isopropyl myristate, or a combination thereof.

In some embodiments, the organic liquid includes an oil (e.g., a plant-based oil or an animal-based oil). In some embodiments, the oil includes coconut oil, cottonseed oil, fish oil, grape seed oil, hazelnut oil, hydrogenated vegetable oils, lime oil, olive oil, palm seed oil, peanut oil, peppermint oil, safflower oil, sesame oil, soybean oil, sunflower oil, walnut oil, or any combination thereof. In some embodiments, the oil includes sesame oil. In some embodiments, the oil includes safflower oil.

In some embodiments, the organic solvent includes an alkane or a perfluoroalkane. In some embodiments, the organic solvent includes perfluorohexyl octane, perfluoro octane, octane, perfluoro decalin, perfluoro butyl pentane, tetradecane, and any combination thereof.

In some embodiments, the organic solvent includes ethyl lactate, ethyl acetate, propyl acetate, or any combination thereof.

Organic liquids of interest include, but are not limited to, benzyl benzoate, ethyl oleate, triacetin, dimethylacetamide (DMAc), Miglyol 840, Miglyol 829, Miglyol 8810, Miglyol 812, Miglyol 812, and the like.

In some embodiments, the organic liquid includes a blend of or organic liquids (e.g., as described herein), e.g., a blend of organic solvents. In some embodiments, the organic liquid is a blend comprising an organic selected from triacylglyceride, diacylglyceride, monoacylglyceride, an acetamide, alkyl alcohol, aryl alcohol, aralkyl alcohol, fatty acid or a fatty acid ester, oil, alkane, perfluoroalkane, propylene glycol monoester, propylene glycol diester, butylene glycol monoester, butylene glycol diester, and polyethylene glycol diester. In some embodiments, the organic liquid blend includes an additional organic solvent. In some embodiments, the organic liquid is a blend of two or more organic liquids independently selected from triacylglyceride, diacylglyceride, monoacylglyceride, an acetamide, alkyl alcohol, aryl alcohol, aralkyl alcohol, fatty acid or a fatty acid ester, oil, alkane, perfluoroalkane, propylene glycol monoester, propylene glycol diester, butylene glycol monoester, butylene glycol diester, and polyethylene glycol diester.

In some embodiments, the organic liquid blend has a lower viscosity than one or more of the individual liquids or solvents in the blend alone. In some embodiments, the viscosity of the organic liquid blend is 25 cp or less at 25° C., such as 23 cp or less, 20 cp or less, 18 cp or less, 15 cp or less, 12 cp or less, 10 cp or less, 8 cp or less, 5 cp or less, or even less. In some embodiments, the organic liquid blend comprises a blend of a triacylglyceride and one or more organic solvents that is less viscous than the triacylglyceride. In some embodiments, the triacylglyceride is triacetin. In some embodiments, the organic liquid blend comprises a blend of a propylene glycol and one or more organic solvents that is less viscous than the propylene glycol. In some embodiments, the propylene glycol is propylene glycol diester or a propylene glycol monoester. In some embodiments, the propylene glycol diester or monoester includes esters that are derived from oleic acid, myristic acid, caprylic acid, capric acid, or any combination thereof. In some embodiments, the ester is an (C₄-C₂₀)-alkyl or substituted (C₄-C₂₀)-alkyl ester of a fatty acid. In some embodiments, the propylene glycol diester or monoester includes diesters of caprylic acid and capric acid (e.g., propylene glycol, dicaprylate, dicaprate).

In some embodiments, the organic liquid blend comprises from 50-90% v/v triacetin and from 10-50% v/v of one or more organic solvents that is less viscous than triacetin. In some embodiments, the organic liquid blend comprises 50-90% v/v triacetin, such as 50%, 55%, 60%, 65%, 70%, 75%, 80%, 85% or 90% v/v triacetin. In some embodiments, the organic liquid blend comprises 10-50% v/v of one or more organic solvents that is less viscous than triacetin, such as 10%, 15%, 20%, 25%, 30%, 35%, 40%, 45%, or 50% v/v of one or more organic solvents that is less viscous than triacetin. In some embodiments, the organic liquid blend comprises 50% v/v triacetin and 50% v/v of one or more organic solvents that is less viscous than triacetin. In some embodiments, the organic liquid blend comprises 55% v/v triacetin and 45% v/v of one or more organic solvents that is less viscous than triacetin. In some embodiments, the organic liquid blend comprises 60% v/v triacetin and 40% v/v of one or more organic solvents that is less viscous than triacetin. In some embodiments, the organic liquid blend comprises 65% v/v triacetin and 35% v/v of one or more organic solvents that is less viscous than triacetin. In some embodiments, the organic liquid blend comprises 70% v/v triacetin and 30% v/v of one or more organic solvents that is less viscous than triacetin. In some embodiments, the organic liquid blend comprises 75% v/v triacetin and 25% v/v of one or more organic solvents that is less viscous than triacetin. In some embodiments, the organic liquid blend comprises 80% v/v triacetin and 20% v/v of one or more organic solvents that is less viscous than triacetin. In some embodiments, the organic liquid blend comprises 85% v/v triacetin and 15% v/v of one or more organic solvents that is less viscous than triacetin. In some embodiments, the organic liquid blend comprises 90% v/v triacetin and 10% v/v of one or more organic solvents that is less viscous than triacetin.

In some embodiments, the organic liquid blend comprises from 50-90% v/v a propylene glycol and from 10-50% v/v of one or more organic solvents that is less viscous than the propylene glycol. In some embodiments, the organic liquid blend comprises 50-90% v/v propylene glycol, such as 50%, 55%, 60%, 65%, 70%, 75%, 80%, 85% or 90% v/v propylene glycol. In some embodiments, the organic liquid blend comprises 10-50% v/v of one or more organic solvents that is less viscous than propylene glycol, such as 10%, 15%, 20%, 25%, 30%, 35%, 40%, 45%, or 50% v/v of one or more organic solvents that is less viscous that triacetin. In some embodiments, the organic liquid blend comprises 50% v/v propylene glycol and 50% v/v of one or more organic solvents that is less viscous than propylene glycol. In some embodiments, the organic liquid blend comprises 55% v/v propylene glycol and 45% v/v of one or more organic solvents that is less viscous than propylene glycol. In some embodiments, the organic liquid blend comprises 60% v/v propylene glycol and 40% v/v of one or more organic solvents that is less viscous than propylene glycol. In some embodiments, the organic liquid blend comprises 65% v/v propylene glycol and 35% v/v of one or more organic solvents that is less viscous than propylene glycol. In some embodiments, the organic liquid blend comprises 70% v/v propylene glycol and 30% v/v of one or more organic solvents that is less viscous than propylene glycol. In some embodiments, the organic liquid blend comprises 75% v/v propylene glycol and 25% v/v of one or more organic solvents that is less viscous than propylene glycol. In some embodiments, the organic liquid blend comprises 80% v/v propylene glycol and 20% v/v of one or more organic solvents that is less viscous than propylene glycol. In some embodiments, the organic liquid blend comprises 85% v/v propylene glycol and 15% v/v of one or more organic solvents that is less viscous than propylene glycol. In some embodiments, the organic liquid blend comprises 90% v/v propylene glycol and 10% v/v of one or more organic solvents that is less viscous than propylene glycol.

In some embodiments, the organic liquid blend comprises triacetin and an organic solvent that is less viscous than triacetin. In some embodiments, the organic liquid comprises triacetin and two or more organic solvents that are less viscous than triacetin. In some embodiments, the one or more less viscous (than triacetin) solvents is selected from DMAc, benzyl benzoate, benzyl alcohol, ethanol, isopropyl alcohol, ethyl lactate, perfluorohexyl octane, perfluoro octane, octane, perfluoro decalin, perfluoro butyl pentane, methoxyflurane, octanol, ethyl acetate, propyl acetate, ethyl oleate and isopropyl myristate. In some embodiments, the one or more less viscous (than triacetin) solvents is selected from DMAc, benzyl benzoate, and benzyl alcohol. In some embodiments, the solvent that is less viscous than triacetin is DMAc. In some embodiments, the solvent that is less viscous than triacetin is benzyl benzoate. In some embodiments, the solvent that is less viscous than triacetin is benzyl alcohol. In some embodiments, the solvent that is less viscous than triacetin includes a combination of DMAc and benzyl alcohol. As such, in some embodiments, the organic liquid comprises a blend of triacetin, DMAc and benzyl alcohol. In some embodiments, the solvent that is less viscous than triacetin includes a 2:1 to 3:1 v/v ratio of DMAc to benzyl alcohol. In some embodiments, the solvent that is less viscous than triacetin includes a combination of DMAc and benzyl benzoate. In some embodiments, the solvent that is less viscous than triacetin includes a 2:1 to 3:1 v/v ratio of DMAc to benzyl benzoate. In some embodiments, the benzyl alcohol is present in an amount of 10% v/v or less of the total volume of the organic liquid.

In some embodiments, the organic liquid includes a blend of a triacylglyceride and an acetamide. In some embodiments the triacylglyceride is triacetin. In some embodiments, the acetamide is DMAc. In some embodiments, the organic liquid comprises a 1:1 to 9:1 volume to volume (v/v) ratio of triacetin to DMAc, such as a 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, or 9:1 v/v ratio of triacetin to DMAc. In some embodiments, the organic liquid comprises a 1:1 to 3:1 v/v ratio of triacetin to DMAc, such as 1:1, 1.2:1, 1.4:1, 1.6:1, 1.8:1, 2:1, 2.2:1, 2.4:1, 2.6:1, 2.8:1, or 3:1 v/v of triacetin to DMAc. In some embodiments, the organic liquid comprises a 3:1 to 5:1 v/v ratio of triacetin to DMAc, such as 3:1, 3.2:1, 3.4:1, 3.6:1, 3.8:1, 4:1, 4.2:1, 4.4:1, 4.6:1, 4.8:1, or 5:1 v/v of triacetin to DMAc. In some embodiments, the organic liquid comprises a 5:1 to 7:1 v/v ratio of triacetin to DMAc, such as 5:1, 5.2:1, 5.4:1, 5.6:1, 5.8:1, 6:1, 6.2:1, 6.4:1, 6.6:1, 6.8:1, or 7:1 v/v of triacetin to DMAc. In some embodiments, the organic liquid comprises a 7:1 to 9:1 v/v ratio of triacetin to DMAc, such as 7:1, 7.2:1, 7.4:1, 7.6:1, 7.8:1, 8:1, 8.2:1, 8.4:1, 8.6:1, 8.8:1, or 9:1 v/v of triacetin to DMAc.

In some embodiments, the organic liquid comprises a 1:1 v/v ratio of triacetin to DMAc. In some embodiments, the organic liquid comprises a 3:1 v/v ratio of triacetin to DMAc. In some embodiments, the organic liquid comprises a 9:1 v/v ratio of triacetin to DMAc.

In some embodiments, the organic liquid includes a blend of a triacylglyceride and aralkyl alcohol. In some embodiments, the triacylglyceride is triacetin. In some embodiments, the aralkyl alcohol is benzyl alcohol. In some embodiments, the organic liquid comprises a 1:1 to 9:1 volume to volume (v/v) ratio of triacetin to benzyl alcohol, such as a 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, or 9:1 v/v ratio of triacetin to benzyl alcohol. In some embodiments, the organic liquid comprises a 1:1 to 3:1 v/v ratio of triacetin to benzyl alcohol, such as 1:1, 1.2:1, 1.4:1, 1.6:1, 1.8:1, 2:1, 2.2:1, 2.4:1, 2.6:1, 2.8:1, or 3:1 v/v of triacetin to benzyl alcohol. In some embodiments, the organic liquid comprises a 3:1 to 5:1 v/v ratio of triacetin to benzyl alcohol, such as 3:1, 3.2:1, 3.4:1, 3.6:1, 3.8:1, 4:1, 4.2:1, 4.4:1, 4.6:1, 4.8:1, or 5:1 v/v of triacetin to benzyl alcohol. In some embodiments, the organic liquid comprises a 5:1 to 7:1 v/v ratio of triacetin to benzyl alcohol, such as 5:1, 5.2:1, 5.4:1, 5.6:1, 5.8:1, 6:1, 6.2:1, 6.4:1, 6.6:1, 6.8:1, or 7:1 v/v of triacetin to benzyl alcohol. In some embodiments, the organic liquid comprises a 7:1 to 9:1 v/v ratio of triacetin to benzyl alcohol, such as 7:1, 7.2:1, 7.4:1, 7.6:1, 7.8:1, 8:1, 8.2:1, 8.4:1, 8.6:1, 8.8:1, or 9:1 v/v of triacetin to benzyl alcohol.

In some embodiments, the organic liquid includes a blend of a triacylglyceride and aralkyl benzoate. In some embodiments the triacylglyceride is triacetin. In some embodiments, the aralkyl benzoate is benzyl benzoate. In some embodiments, the organic liquid comprises a 1:1 to 9:1 volume to volume (v/v) ratio of triacetin to benzyl benzoate, such as a 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, or 9:1 v/v ratio of triacetin to benzyl benzoate. In some embodiments, the organic liquid comprises a 1:1 to 3:1 v/v ratio of triacetin to benzyl benzoate, such as 1:1, 1.2:1, 1.4:1, 1.6:1, 1.8:1, 2:1, 2.2:1, 2.4:1, 2.6:1, 2.8:1, or 3:1 v/v of triacetin to benzyl benzoate. In some embodiments, the organic liquid comprises a 3:1 to 5:1 v/v ratio of triacetin to benzyl benzoate, such as 3:1, 3.2:1, 3.4:1, 3.6:1, 3.8:1, 4:1, 4.2:1, 4.4:1, 4.6:1, 4.8:1, or 5:1 v/v of triacetin to benzyl benzoate. In some embodiments, the organic liquid comprises a 5:1 to 7:1 v/v ratio of triacetin to benzyl benzoate, such as 5:1, 5.2:1, 5.4:1, 5.6:1, 5.8:1, 6:1, 6.2:1, 6.4:1, 6.6:1, 6.8:1, or 7:1 v/v of triacetin to benzyl benzoate. In some embodiments, the organic liquid comprises a 7:1 to 9:1 v/v ratio of triacetin to benzyl benzoate, such as 7:1, 7.2:1, 7.4:1, 7.6:1, 7.8:1, 8:1, 8.2:1, 8.4:1, 8.6:1, 8.8:1, or 9:1 v/v of triacetin to benzyl benzoate.

In some embodiments, the composition includes a 1:5 to 4:1 weight to weight ratio of the liquid carrier to the particles. In some embodiments, the composition includes a 1:1 to 2:1 weight to weight ratio of the liquid carrier to the particles. In some embodiments, the composition includes a 1:1 to 1.5:1 weight to weight ratio of the liquid carrier to the particles.

In some embodiments, the composition includes 200 mg/mL to 850 mg/mL of particles in the liquid carrier. In some embodiments, the composition includes 300 mg/mL to 500 mg/mL of particles in the liquid carrier. In some embodiments, the composition includes 400 mg/mL to 600 mg/mL, such as 450 mg/mL to 550 mg/mL of particles in the liquid carrier.

### 4.3. Loaded Syringe

Also provided in the present disclosure is a syringe loaded with an injectable pharmaceutical composition (e.g., as described herein), for example, a syringe that is pre-loaded with an injectable pharmaceutical composition as described herein in advance of dispensing of the composition from the syringe.

In some embodiments, the loaded syringe is configured to dispense the composition at a flow rate of 0.1 mL/min or more, such as 0.5 mL/min or more, 1 mL/min or more, 2 mL/min or more, 5 mL/min or more, 10 mL/min or more, 15 mL/min or more, or 20 mL/min or more, in response to a force applied to the syringe of 70 N or less. In some embodiments, the syringe is configured to dispense the composition at a flow rate of 0.1 mL/min or more, such as 0.5 mL/min or more, 1 mL/min or more, 2 mL/min or more, 5 mL/min or more, 10 mL/min or more, 15 mL/min or more, or 20 mL/min or more, in response to a force applied to the syringe of 50 N or less.

In some embodiments, the loaded syringe has a needle having a size of 18 to 32 Gauge (G). In some embodiments, the needle is an 18 G needle. In some embodiments, the needle is a 21 G needle. In some embodiments, the needle is a 22 G needle. In some embodiments, the needle is a 24 G needle. In some embodiments, the needle is a 25 G needle. In some embodiments, the needle is a 26 G needle. In some embodiments, the needle is a 27 G needle. In some embodiments, the needle is a 30 G needle. In some embodiments, the needle is a 32 G needle.

In some embodiments of any of the needles described herein, the needle is an ultra-thin-walled needle (UTW). It is understood that the UTW needle can have an inner diameter that is larger than a conventional gauge needle.

### 4.4. Methods of Administration

Also provided in the present disclosure are methods of administering a biopharmaceutical to a subject by injection. In some embodiments, the method includes injecting a composition (e.g., as described herein) in a subject in need thereof to administer a therapeutically effective dose of the biopharmaceutical agent to the subject.

In some embodiments of the method, the injecting is performed using a loaded syringe, such as a pre-loaded syringe, that includes the composition.

### 4.5. Methods of Preparation

As summarized herein, also provided are methods of preparing an injectable pharmaceutical composition (e.g., as described herein). In some embodiments, the method of preparing an injectable pharmaceutical composition includes:
a) providing a mixture comprising:
   a biopharmaceutical agent; and
   a polyacrylamide-based copolymer in water;
b) removing the water from the mixture to obtain particles comprising the biopharmaceutical agent and the polyacrylamide-based copolymer; and
c) contacting a liquid carrier with the particles to form a suspension of the particles in the organic liquid.

In some embodiments of the method, in step b) the water is removed via spray drying. In some embodiments of the method, in step b) the water is removed via lyophilization. In some embodiments of the method, in step b) the water is removed via electrospray drying.

In some embodiments, the method further includes a milling step after the lyophilization but prior to step c).

In some embodiments, the particles obtained in step b) have a mean diameter as described herein, e.g., of 100 microns or less.

In some embodiments, step c) of the method includes sonicating the particles in the liquid carrier.

### 4.6. Definitions

Unless otherwise defined, all technical and scientific terms used herein have the same meaning as commonly understood by one of ordinary skill in the art to which this disclosure belongs. Methods and materials are described herein for use in the present application; other, suitable methods and materials known in the art in some aspects of this disclosure are also used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control. When trade names are used herein, the trade name includes the product formulation, the generic drug, and the active pharmaceutical ingredient(s) of the trade name product, unless otherwise indicated by context.

The terms “subject” and “patient” are used interchangeably. A subject can be a mammal such as a non-primate (e.g., cows, pigs, horses, cats, dogs, goats, rabbits, rats, mice, etc.) or a primate (e.g., monkey, ape, and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” are used in their broadest sense understood in the clinical arts.

In this disclosure, the terms “a,” “an,” and “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about” as used in the present disclosure can allow for a degree of variability in a value or range that is within 5% of a stated value or of a stated limit of a range.

In the methods described in the present disclosure, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “polymer” refers to a substance or material consisting of repeating monomer subunits.

An “acrylamide monomer,” as used herein, refers to a monomer species that possesses an acrylamide functional group. The term “acrylamide monomer” includes not only monomeric acrylamide, but derivatives of monomeric acrylamide. Examples of acrylamide monomers include, but are not limited to, acrylamide (AM), N-(3-methoxypropoyl)acrylamide (MPAM), 4-acryloylmorpholine (MORPH), N,N-dimethylacrylamide (DMA), N-hydroxyethyl acrylamide (HEAM), N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N,N-diethylacrylamide (DEA), N-tert-butylacrylamide (TBA), and N-phenylacrylamide (PHE).

The term “polyacrylamide-based copolymer” refers to polymers that are formed from the polymerization of two or more monomer species, in which at least one of the monomer species possesses an acrylamide functional group (acrylamide monomer) and the monomers are structurally different. In some embodiments, the polyacrylamide-based copolymer is formed from the polymerization of two structurally different acrylamide monomers (two structurally different monomers that each possess an acrylamide functional group). The resulting copolymer can be an alternating copolymer wherein the monomer species are connected in an alternating fashion; a random copolymer, wherein the monomer species are connected to each other within a polymer chain without a defined pattern; a block copolymer, wherein polymeric blocks of one monomer species are connected to polymeric blocks made up of another monomer species; and graft copolymer, wherein the main polymer chain consists of one monomer species, and polymeric blocks of another monomer species are connected to the main polymer chain as side branches. In some embodiments, the polyacrylamide-based copolymers of the present disclosure are formed from the polymerization of a water-soluble carrier monomer and a functional dopant monomer. In some embodiments, the polyacrylamide-based copolymers of the present disclosure are random copolymers.

As defined herein, the term “water-soluble carrier monomer” refers to an acrylamide monomer species that is the water-soluble species within the polyacrylamide-based copolymer. In some embodiments, the water-soluble carrier monomer is the predominant species within the polyacrylamide-based copolymer. In some embodiments, the water-soluble carrier monomer imparts aqueous solubility to the copolymer. In some embodiments, the water-soluble carrier monomer within the polyacrylamide-based copolymer provides an inert barrier at the interface of an aqueous formulation to prevent protein-protein interactions. In some embodiments, the interface is an air-water interface. In some embodiments, the interface is an enclosure-water interface, including, but not limited to, a glass-water interface, a rubber-water interface, a plastic-water interface, or a metal-water interface. In some embodiments, the interface is an oil-water interface. In some embodiments, the interface is an interface between a liquid and tubing. In some embodiments, the interface is an interface between a liquid and a catheter. In some embodiments, the enclosure-water interface is in a pump system. In some embodiments, the enclosure-water interface is in a closed-loop system. In some embodiments, the water-soluble carrier monomer is nonionic. Examples of water-soluble carrier monomers include, but are not limited to, acrylamide (AM), N-(3-methoxypropoyl)acrylamide (MPAM), 4-acryloylmorpholine (MORPH), N,N-dimethylacrylamide (DMA), and N-hydroxyethyl acrylamide (HEAM).

The term “functional dopant monomer,” as used herein, refers to an acrylamide monomer species that has physicochemical properties (e.g., hydrophobicity, charge) different from those of the water-soluble carrier monomer. In some embodiments, the functional dopant monomer within the polyacrylamide-based copolymer promotes association of the polymers to an interface; such interfaces can include, but are not limited to, polymer-air-water interface interactions, polymer-protein interactions, polymer-peptide interactions, polymer-micelle interactions, polymer-liposome interactions, and polymer-lipid nanoparticle interactions. The functional dopant monomer can act as a stabilizing moiety to facilitate interactions with biomolecules, for example, proteins, peptides, antibodies, antibody-drug conjugates, nucleic acids, lipid particles, and combinations thereof (e.g., to prevent aggregation of the biomolecules). The functional dopant monomers can be further classified into hydrogen-bonding, ionic, hydrophobic, and aromatic monomers based on their chemical composition. Typically, the functional dopant monomers are copolymerized at a lower weight percentage as compared to the water-soluble carrier monomers.

The term “polymerization” refers to the process in which monomer molecules undergo a chemical reaction to form polymeric chains or three-dimensional networks. Different types of polymerization reactions are known in the art, for example, addition (chain-reaction) polymerization, condensation polymerization, ring-opening polymerization, free radical polymerization, controlled radical polymerization, atom transfer radical polymerization (ATRP), single-electron transfer living radical polymerization (SET-LRP), reversible addition-fragmentation chain transfer (RAFT) polymerization, nitroxide-mediated polymerization (NMP), and emulsion polymerization. In some embodiments, the copolymers of the present disclosure are prepared using RAFT polymerization.

The term “degree of polymerization” (DP) refers to the number of monomer units in a polymer. It is calculated by dividing the average molecular weight of a polymer sample by the molecular weight of the monomers. As defined herein, the average molecular weight of a polymer can be represented by the number-averaged molecular weight (Mn), the weight-average molecular weight (Mw), the Z-average molecular weight (Mz) or the molecular weight at the peak maxima of the molecular weight distribution curve (Mp). The average molecular weight of a polymer can be determined by a variety of analytical characterization techniques known to those skilled in the art, for example, size exclusion chromatography (SEC), static light scattering (SLS) analysis, multi-angle laser light scattering (MALLS) analysis, nuclear magnetic resonance spectroscopy (NMR), intrinsic viscometry (IV), melt flow index (MFI), and matrix-assisted laser desorption/ionization mass spectrometry (MALDI-MS), and combinations thereof. Degree of polymerization can also be determined experimentally using suitable analytical methods known in the art, such as nuclear magnetic spectroscopy (NMR), Fourier Transform infrared spectroscopy (FT-IR) and Raman spectroscopy.

The term “amphiphilic” refers to chemical substances that possess both hydrophilic (water-loving, polar) and lipophilic or hydrophobic (fat-loving, nonpolar) properties. Examples of common amphiphilic compounds include detergents, soaps, surfactants, lipoproteins, and phospholipids. In some embodiments, the amphiphilic substance is a charged species. In some embodiments, the amphiphilic substance is a neutral species. In some embodiments, the co-polymers incorporated into the particles of this disclosure are amphiphilic because they include both a hydrophilic co-monomers, and lipophilic or hydrophobic co-monomers.

A “lipid-based vehicle,” as used herein, refers to structures having a protective outer layer of lipids that can be used as drug delivery vehicles. For example, a lipid-based vehicle can be used to encapsulate and transport cargo (e.g., a therapeutic agent) to a biologic target. Examples of lipid-based vehicles include, but are not limited to, liposomes, micelles, polymerosomes, and lipid nanoparticles.

“Biologic molecule,” as used herein, refers to molecules such as proteins, nucleic acids, polysaccharides, and lipids.

The term “protein” is defined as a class of large molecules comprising one or more long chains of amino acids. A wide variety of proteins may be considered as belonging to a family of proteins based on having similar structural features, having particular biological functions, and/or being related to specific microorganisms, particularly disease-causing microorganisms. Such proteins include, for example, antibodies, cytokines, chemokines, enzymes, hormones, vaccine antigens, cancer antigens, adjuvants, nutritional markers, and tissue specific antigens.

The term “nucleic acid,” as used herein, includes deoxyribonucleic acid (DNA), ribonucleic acid (RNA), messenger RNA (mRNA), small-interfering RNA (siRNA), short hairpin RNA (shRNA), and microRNA (miRNA).

The term “antibody” refers to large, immunoglobulin proteins produced by the immune system to identify and neutralize foreign objects such as pathogenic bacteria and viruses. The term “antibody” includes monoclonal antibodies (for example, full length or intact monoclonal antibodies), polyclonal antibodies, multivalent antibodies, multispecific antibodies (e.g., bispecific or trispecific antibodies, so long as they exhibit the desired biological activity) and can also include certain antibody fragments. An antibody can be human, humanized and/or affinity matured. “Antibody fragments” include only a portion of an intact antibody, where in certain embodiments, the portion retains at least one, and typically most or all, of the functions normally associated with that portion when present in an intact antibody. In one embodiment, an antibody fragment includes an antigen binding site of the intact antibody and thus retains the ability to bind antigen. In another embodiment, an antibody fragment, for example one that includes the Fe region, retains at least one of the biological functions normally associated with the Fe region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may include an antigen binding arm linked to an Fe sequence capable of conferring in vivo stability to the fragment.

The term “aggregation” refers to the formation of higher molecular weight, amorphous species due to non-covalent adherence (“clumping”) of smaller species. The aggregation process can be irreversible or reversible. Many biological and synthetic molecules can undergo aggregation, including proteins, peptides, lipid particles, nucleic acids, inorganic nanoparticles and organic nanoparticles (e.g., micelles, lipid nanoparticles, liposomes, polymerosomes) that may further include an encapsulated species.

In the case of protein aggregation, formation of protein aggregates can be due to the protein's intrinsic disordered nature, or misfolding of protein molecules, which results in the exposure of hydrophobic residues and surfaces that are normally buried within the interior of the protein three-dimensional structure. Due to the hydrophobic effect, the exposed hydrophobic portions of a misfolded protein have the tendency to interact with other misfolded protein molecules to shield the exposed hydrophobic surfaces, which can lead to protein aggregation.

Some biologic molecules are more “susceptible to aggregation” than others. For example, the amino-acid sequence and overall three-dimensional structure of a protein is relevant to its susceptibility to aggregation. For example, transmembrane proteins are more prone (or susceptible) to aggregation than non-membrane proteins, particularly when expressed recombinantly without the use of a stabilizing agent. Proteins that are subject to conditions beyond the physiological conditions (37° C., —neutral pH, isotonic) may also be more susceptible to aggregation than when in their native environment. Stress conditions such as temperature fluctuations, light, mechanical perturbation (e.g., shaking), surfaces, ultrasonic vibration, pH changes, and changes in ionic strength can affect protein stability and induce aggregation. Protein aggregation can lead to the formation of sub-visible or visible particles (i.e., precipitation). The extent of sub-visible protein aggregation can be measured by a variety of analytical methods known in the art, for example, size-exclusion chromatography (SEC), gel electrophoresis, asymmetric field-flow fractionation (AF4), analytical ultracentrifugation (AUC), mass spectrometry (MS), optical microscopy, fluorescence microscopy, dynamic light scattering (DLS), multi-angle laser light scattering (MALLS), flow imaging, turbidity/nephelometry, and transmittance measurement.

As used herein, the term “reduced aggregation” of a biologic molecule or lipid-based vehicle includes all forms of reducing aggregation. The degree or amount of aggregation observed (e.g., in the composition) can be reduced as compared to a composition of the same biologic molecule or lipid-based vehicle in the absence of the polyacrylamide-based copolymer of the present disclosure. Thus, “reduced aggregation” includes no observable aggregation or reduced amounts of aggregation (e.g., reduced levels of aggregated protein). Thus, the amount of aggregates present in the composition can be reduced by at least about 10 mol %, about 20 mol %, about 30 mol %, about 40 mol %, about 50 mol %, about 60 mol %, about 70 mol %, about 80 mol %, about 90 mol %, or about 100 mol % as compared to the amount of aggregates of the same biologic molecule or lipid-based vehicle in the absence of the polyacrylamide-based copolymer. Aggregation can be measured by any method known in the art, including, but not limited to, size-exclusion chromatography (SEC), gel electrophoresis, asymmetric field-flow fractionation (AF4), analytical ultracentrifugation (AUC), mass spectrometry (MS), optical microscopy, fluorescence microscopy, dynamic light scattering (DLS), multi-angle laser light scattering (MALLS), flow imaging, turbidity/nephelometry, and transmittance measurement.

As used herein, the term “increased stability,” when referring to a formulation containing a biologic molecule or lipid-based vehicle, refers to a measurable decrease in the amount of aggregation over a fixed period of time under testing or fixed storage conditions as compared to the amount of aggregates of the same biologic molecule or lipid-based vehicle in the absence of the polyacrylamide-based copolymer.

The terms “aggregated protein” or “protein aggregates” as used herein refer to a collection of proteins that are disordered or misfolded and grouped together. The aggregates can be soluble or insoluble. Protein aggregates include, but are not limited to, inclusion bodies, soluble and insoluble precipitates, soluble non-native oligomers, gels, fibrils, films, filaments, protofibrils, amyloid deposits, amyloid fibrils, plaques, and dispersed non-native intracellular oligomers. In some embodiments, the proteins in a protein aggregate are, prior to their aggregation, soluble precursors. Protein aggregation can be prevented in compositions containing the polyacrylamide-based copolymer of the present disclosure. Protein aggregation can also be reduced in a composition containing the polyacrylamide-based copolymer of the present disclosure as compared to a composition containing the same protein that does not contain the polyacrylamide-based copolymer of the present disclosure. Thus, the polyacrylamide-based copolymer can reduce or prevent the aggregation of a protein.

### 5. ADDITIONAL EMBODIMENTS

This disclosure is further described by the following non-limiting clauses:
1. An injectable pharmaceutical composition comprising:
   particles, the particles comprising:
      a biopharmaceutical agent; and
      a polyacrylamide-based copolymer; and
   a liquid carrier in which the particles are suspended.
2. The injectable pharmaceutical composition of clause 1, wherein the particles have a mean diameter of 100 microns or less.
3. The injectable pharmaceutical composition of clause 2, wherein the particles have a mean diameter of from 0.01-100 microns.
4. The injectable pharmaceutical composition of clause 3, wherein the particles have a mean diameter of from 0.1-100 microns.
5. The injectable pharmaceutical composition of clause 4, wherein the particles have a mean diameter of from 0.2-20 microns.
6. The injectable pharmaceutical composition of clause 5, wherein the particles have a mean diameter of from 0.2-10 microns.
7. The injectable pharmaceutical composition of clause 1, further comprising one or more of stabilizing agent, preservative, filler, bulking agent, sugar, polysaccharide, or viscosity modifier.
8. The injectable pharmaceutical composition of clause 7, further comprising a stabilizing agent.
9. The injectable pharmaceutical composition of clause 8, wherein the stabilizing agent is selected from surfactants, poloxamers, povidones, polyvinylpyrrolidone (PVP) polymer, polyvinyl alcohol (PVA) polymer, polysaccharides, cellulosics, amphoteric compounds, sugars, salts, and combinations thereof.
10. The injectable pharmaceutical composition of any one of clauses 1 to 9, wherein the biopharmaceutical agent is a polypeptide.
11. The injectable pharmaceutical composition of clause 10, wherein the polypeptide is susceptible to aggregation in an aqueous medium.
12. The injectable pharmaceutical composition of clause 10 or 11, wherein the polypeptide is selected from antibodies and fragments thereof, cytokines, chemokines, hormones, vaccine antigens, cancer antigens, adjuvants, and combinations thereof.
13. The injectable pharmaceutical composition of any one of clauses 10 to 12, wherein the polypeptide is a protein.
14. The injectable pharmaceutical composition of clause 13, wherein the protein is an antibody or a fragment thereof.
15. The injectable pharmaceutical composition of clause 14, wherein the protein is a monoclonal antibody, a polyclonal antibody, an immunoglobulin G (IgG) antibody, an IgA antibody, an IgM antibody, a Fc fusion protein, or a fragment thereof.
16. The injectable pharmaceutical composition of any one of clauses 10 to 12, wherein the polypeptide is a hormone or analog thereof.
17. The injectable pharmaceutical composition of clause 16, wherein the polypeptide is insulin or an analog thereof.
18. The injectable pharmaceutical composition of clause 16, wherein the polypeptide is selected from glucagon, GLP-1 receptor agonist, amylin, and analogs thereof.
19. The injectable pharmaceutical composition of any one of clauses 1 to 18, wherein the composition comprises 0.1 to 20% by weight of the biopharmaceutical agent.
20. The injectable pharmaceutical composition of clause 19, wherein the biopharmaceutical agent is insulin or an analog thereof.
21. The injectable pharmaceutical composition of clause 19, wherein the biopharmaceutical agent is a peptide.
22. The injectable pharmaceutical composition of any one of clauses 19 to 21, wherein the particles comprise no more than 20% by weight of the biopharmaceutical agent.
23. The injectable pharmaceutical composition of any one of clauses 1 to 18, wherein the composition comprises 20% or more by weight of the biopharmaceutical agent.
24. The injectable pharmaceutical composition of clause 23, wherein the composition comprises 20% to 80% by weight of the biopharmaceutical agent.
25. The injectable pharmaceutical composition of clause 23 or 24, wherein the biopharmaceutical agent is an antibody or fragment thereof, or a Fc fusion protein.
26. The injectable pharmaceutical composition of any one of clauses 23 to 25, wherein the particles comprise 75% or more by weight of the biopharmaceutical agent.
27. The injectable pharmaceutical composition of any one of clauses 1 to 26, wherein the composition comprises 0.01 to 20 wt % of the polyacrylamide-based copolymer.
28. The injectable pharmaceutical composition of clause 27, wherein the composition comprises 0.1 to 10 wt % of the polyacrylamide-based copolymer.
29. The injectable pharmaceutical composition of clause 27, wherein the composition comprises 0.5 to 5 wt % of the polyacrylamide-based copolymer.
30. The injectable pharmaceutical composition of any one of clauses 1 to 29, wherein the particles comprise 0.01 to 25 wt % of the polyacrylamide-based copolymer.
31. The injectable pharmaceutical composition of clause 30, wherein the particles comprise 0.1 to 10 wt % of the polyacrylamide-based copolymer.
32. The injectable pharmaceutical composition of clause 30, wherein the particles comprise 1 to 5 wt % of the polyacrylamide-based copolymer.
33. The injectable pharmaceutical composition of any one of clauses 1 to 32, wherein composition comprises a 1:500 to 2:1 weight to weight ratio of the polyacrylamide-based copolymer to the biopharmaceutical agent.
34. The injectable pharmaceutical composition of clause 33, wherein composition comprises a 1:50 to 1:1 weight to weight ratio of the polyacrylamide-based copolymer to the biopharmaceutical agent.
35. The injectable pharmaceutical composition of clause 33, wherein composition comprises a 1:25 to 1:10 weight to weight ratio of the polyacrylamide-based copolymer to the biopharmaceutical agent.
36. The injectable pharmaceutical composition of any one of clauses 1 to 35, wherein the polyacrylamide-based copolymer comprises:
   a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof; and
   a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof.
37. The injectable pharmaceutical composition of clause 33, wherein the water-soluble carrier monomer is selected from MORPH, MPAM, and combinations thereof.
38. The injectable pharmaceutical composition of clause 34, wherein the water-soluble carrier monomer comprises MORPH.
39. The injectable pharmaceutical composition of clause 34, wherein the water-soluble carrier monomer comprises MPAM.
40. The injectable pharmaceutical composition of any one of clauses 33-36, wherein the functional dopant monomer is selected from AMP, TMA, TBA, PHE, and combinations thereof.
41. The injectable pharmaceutical composition of any one of clauses 33-36, wherein the functional dopant monomer is selected from DEA, PHE, NIP, and combinations thereof.
42. The injectable pharmaceutical composition of any one of clauses 33-36, wherein the functional dopant monomer comprises TRI.
43. The injectable pharmaceutical composition of any one of clauses 33-36, wherein the functional dopant monomer comprises PHE.
44. The injectable pharmaceutical composition of any one of clauses 33-36, wherein the functional dopant monomer comprises NIP.
45. The injectable pharmaceutical composition of any one of clauses 33-36, wherein the functional dopant monomer comprises DEA.
46. The injectable pharmaceutical composition of clause 33, wherein:
   the water-soluble carrier monomer is selected from MPAM, MORPH, and combinations thereof; and
   the functional dopant monomer is selected from NIP, PHE, and combinations thereof.
47. The injectable pharmaceutical composition of clause 33, wherein:
   the water-soluble carrier monomer is selected from MPAM, MORPH, and combinations thereof; and
   the functional dopant monomer is selected from AMP, TMA, TBA, PHE, and combinations thereof.
48. The injectable pharmaceutical composition of clause 33, wherein the water-soluble carrier monomer is MPAM, and the functional dopant monomer is PHE.
49. The injectable pharmaceutical composition of clause 33, wherein the water-soluble carrier monomer is MORPH, and the functional dopant monomer is PHE.
50. The injectable pharmaceutical composition of clause 33, wherein the water-soluble carrier monomer is MORPH, and the functional dopant monomer is NIP.
51. The injectable pharmaceutical composition of any one of clauses 33-50, wherein the polyacrylamide-based copolymer comprises:
   70 wt % to 98 wt % of the water-soluble carrier monomer; and
   2 wt % to 30 wt % of the functional dopant monomer.
52. The injectable pharmaceutical composition of any one of clauses 33-50, wherein the polyacrylamide-based copolymer comprises:
   80 wt % to 95 wt % of the water-soluble carrier monomer; and
   5 wt % to 20 wt % of the functional dopant monomer.
53. The injectable pharmaceutical composition of any one of clauses 33-50, wherein the polyacrylamide-based copolymer comprises:
   83 wt % to 98 wt % of the water-soluble carrier monomer; and
   2 wt % to 17 wt % of the functional dopant monomer.
54. The injectable pharmaceutical composition of clause 33, wherein the polyacrylamide-based copolymer comprises:
   70 wt % to 85 wt % of MORPH; and
   15 wt % to 30 wt % of NIP.
55. The injectable pharmaceutical composition of clause 33, wherein the polyacrylamide-based copolymer comprises:
   74 wt % to 80 wt % of MORPH; and
   20 wt % to 26 wt % of NIP.
56. The injectable pharmaceutical composition of clause 33, wherein the polyacrylamide-based copolymer comprises:
   77 wt % of MORPH; and
   23 wt % of NIP.
57. The injectable pharmaceutical composition of any one of clauses 33-56, wherein the degree of polymerization of the polyacrylamide-based copolymer is 10 to 500.
58. The injectable pharmaceutical composition of clause 57, wherein the degree of polymerization of the polyacrylamide-based copolymer is 20 to 200.
59. The injectable pharmaceutical composition of clause 58, wherein the degree of polymerization of the polyacrylamide-based copolymer is 50.
60. The injectable pharmaceutical composition of any one of clauses 22-59, wherein a number-average molecular weight of the polyacrylamide-based copolymer is 1,000 g/mol to 40,000 g/mol.
61. The injectable pharmaceutical composition of 60, wherein a number-average molecular weight of the polyacrylamide-based copolymer is 2,000 g/mol to 10,000 g/mol.
62. The injectable pharmaceutical composition of 61, wherein a number-average molecular weight of the polyacrylamide-based copolymer is 4,000 g/mol to 6,000 g/mol.
63. The injectable pharmaceutical composition of any one of clauses 1 to 62, wherein the polyacrylamide-based copolymer is amphiphilic.
64. The injectable pharmaceutical composition of any one of clauses 1 to 63, wherein the liquid carrier comprises a mixture of an organic solvent and an aqueous solution.
65. The injectable pharmaceutical composition of any one of clauses 1 to 63, wherein the liquid carrier comprises an organic solvent, an oil, or a combination thereof.
66. The injectable pharmaceutical composition of any one of clauses 1 to 65, wherein the liquid carrier comprises one or more liquids selected from triacylglyceride, diacylglyceride, monoacylglyceride, an acetamide, alkyl alcohol, aryl alcohol, aralkyl alcohol, fatty acid, fatty acid ester, oil, alkane, perfluoroalkane, propylene glycol monoester, propylene glycol diester, butylene glycol monoester, butylene glycol diester, polyethylene glycol diester, and combinations thereof.
67. The injectable pharmaceutical composition of clause 66, wherein the liquid carrier comprises an aralkyl benzoate.
68. The injectable pharmaceutical composition of clause 67, wherein the aralkyl benzoate is benzyl benzoate.
69. The injectable pharmaceutical composition of clause 66, wherein the liquid carrier comprises a triacylglyceride, diacylglyceride, monoacylglyceride, or a combination thereof.
70. The injectable pharmaceutical composition of clause 69, wherein the liquid carrier comprises a triacylglyceride that is triacetin.
71. The injectable pharmaceutical composition of clause 66, wherein the liquid carrier comprises N,N-dimethylacetamide (DMAc).
72. The injectable pharmaceutical composition of clause 66, wherein the liquid carrier comprises a fatty acid ester.
73. The injectable pharmaceutical composition of clause 72, wherein the fatty acid ester is ethyl oleate.
74. The injectable pharmaceutical composition of clause 66, wherein the liquid carrier comprises a propylene glycol diester and/or a butylene glycol diester.
75. The injectable pharmaceutical composition of clause 74, wherein the liquid carrier comprises Miglyol 840.
76. The injectable pharmaceutical composition of clause 65 or 66, wherein the liquid carrier comprises a blend of a triacylglyceride and one or more organic solvents that is less viscous than the triacylglyceride.
77. The injectable pharmaceutical composition of clause 76, wherein the triacylglyceride is triacetin.
78. The injectable pharmaceutical composition of clause 77, wherein the liquid carrier comprises a 1:1 to 9:1 volume to volume (v/v) ratio of triacetin to one or more organic solvents that is less viscous than triacetin.
79. The injectable pharmaceutical composition of clause 76, wherein the liquid carrier comprises a 1:1 v/v ratio of triacetin to one or more organic solvents that is less viscous than triacetin.
80. The injectable pharmaceutical composition of clause 76, wherein the liquid carrier comprises a 3:1 v/v ratio of triacetin to one or more organic solvents that is less viscous than triacetin.
81. The injectable pharmaceutical composition of clause 76, wherein the liquid carrier comprises a 9:1 v/v ratio of triacetin to one or more organic solvents that is less viscous than triacetin.
82. The injectable pharmaceutical composition of any one of clauses 76 to 81, wherein the organic solvent that is less viscous that triacetin is selected from an acetamide, an alkyl benzoate, an aryl benzoate, an aralkyl benzoate, an aryl alcohol, an aralkyl alcohol, or any combination thereof.
83. The injectable pharmaceutical composition of clause 82, wherein the organic solvent that is less viscous that triacetin is an acetamide.
84. The injectable pharmaceutical composition of clause 83, wherein the acetamide is DMAc.
85. The injectable pharmaceutical composition of clause 82, wherein the organic solvent that is less viscous that triacetin is an aralkyl alcohol.
86. The injectable pharmaceutical composition of clause 85, wherein the aralkyl alcohol is benzyl alcohol.
87. The injectable pharmaceutical composition of clause 82, wherein the organic solvent that is less viscous that triacetin is an aralkyl benzoate.
88. The injectable pharmaceutical composition of clause 87, wherein the aralkyl benzoate is benzyl benzoate.
89. The injectable pharmaceutical composition of clause 82, wherein the organic solvent that is less viscous that triacetin comprises a combination of an acetamide and an aralkyl alcohol.
90. The injectable pharmaceutical composition of clause 89, wherein the acetamide is DMAc, and the aralkyl alcohol is benzyl alcohol.
91. The injectable pharmaceutical composition of clause 90, wherein the less viscous organic solvent comprises a 3:1 to 2:1 v/v ratio of DMAc to benzyl alcohol.
92. The injectable pharmaceutical composition of clause 65 or 66, wherein the organic solvent is an alkyl benzoate, an aryl benzoate, an aralkyl benzoate, or any combination thereof.
93. The injectable pharmaceutical composition of clause 92, wherein the organic solvent is benzyl benzoate.
94. The injectable pharmaceutical composition of clause 64 or 65, wherein the organic solvent is a polar aprotic solvent.
95. The injectable pharmaceutical composition of clause 94, wherein the polar aprotic solvent is dimethyl sulfoxide (DMSO), N-methyl-2-pyrrolidone (NMP), or a mixture thereof.
96. The injectable pharmaceutical composition of clause 65, wherein the liquid carrier comprises an oil (e.g., a plant-based oil or an animal-based oil).
97. The injectable pharmaceutical composition of clause 96, wherein the oil is coconut oil, cottonseed oil, fish oil, grape seed oil, hazelnut oil, hydrogenated vegetable oils, lime oil, olive oil, palm seed oil, peanut oil, peppermint oil, safflower oil, sesame oil, soybean oil, sunflower oil, walnut oil, or any combination thereof.
98. The injectable pharmaceutical composition of clause 97, wherein the oil is sesame oil.
99. The injectable pharmaceutical composition of any one of clauses 1 to 98, wherein the composition comprises a 1:5 to 4:1 weight to weight ratio of the liquid carrier to the particles.
100. The injectable pharmaceutical composition of clause 99, wherein the composition comprises a 1:1 to 2:1 weight to weight ratio of the liquid carrier to the particles.
101. An injectable pharmaceutical composition comprising:
   particles, the particles comprising:
      a biopharmaceutical agent; and
      a polyacrylamide-based copolymer comprising:
         a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof; and
         a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof; and
   a liquid carrier in which the particles are suspended, wherein the liquid carrier is selected from triacylglyceride, diacylglyceride, monoacylglyceride, an acetamide, alkyl alcohol, aryl alcohol, aralkyl alcohol, fatty acid, fatty acid ester, oil, alkane, perfluoroalkane, propylene glycol monoester, propylene glycol diester, butylene glycol monoester, butylene glycol diester, polyethylene glycol diester, and combinations thereof.
102. The injectable pharmaceutical composition of clause 101, wherein the water-soluble carrier monomer is selected from MORPH, MPAM, and combinations thereof.
103. The injectable pharmaceutical composition of any one of clauses 101-102, wherein the functional dopant monomer is selected from AMP, TMA, TBA, PHE, and combinations thereof. 104. The injectable pharmaceutical composition of any one of clauses 101-102, wherein the functional dopant monomer is selected from DEA, PHE, NIP, and combinations thereof.
105. The injectable pharmaceutical composition of clause 104, wherein:
   the water-soluble carrier monomer is selected from MPAM, MORPH, and combinations thereof; and
   the functional dopant monomer is selected from NIP, PHE, and combinations thereof.
106. The injectable pharmaceutical composition of clause 104, wherein the water-soluble carrier monomer is MORPH, and the functional dopant monomer is NIP.
107. The injectable pharmaceutical composition of any one of clauses 101-106, wherein the liquid carrier comprises an aralkyl benzoate.
108. The injectable pharmaceutical composition of clause 107, wherein the aralkyl benzoate is benzyl benzoate.
109. The injectable pharmaceutical composition of any one of clauses 101-106, wherein the liquid carrier comprises a triacylglyceride.
110. The injectable pharmaceutical composition of clause 109, wherein the triacylglyceride is triacetin.
111. The injectable pharmaceutical composition of any one of clauses 101-106, wherein the liquid carrier comprises N,N-dimethylacetamide (DMAc).
112. The injectable pharmaceutical composition of any one of clauses 101-106, wherein the liquid carrier comprises a fatty acid ester.
113. The injectable pharmaceutical composition of clause 112, wherein the fatty acid ester comprises ethyl oleate.
114. The injectable pharmaceutical composition of any one of clauses 101-106, wherein the liquid carrier comprises a propylene glycol diester and/or a butylene glycol diester.
115. The injectable pharmaceutical composition of clause 114, wherein:
   the propylene glycol diester is Miglyol 840; and
   the butylene glycol diester is Miglyol 8810.
116. A syringe, loaded with a composition of any one of clauses 1-115.
117. The syringe of clause 116, configured to dispense the composition at a flow rate of 0.1 mL/min or more in response to a force applied to the syringe of 70 N or less.
118. The syringe of clause 116, configured to dispense the composition at a flow rate of 0.1 mL/min or more in response to a force applied to the syringe of 50 N or less.
119. The syringe of any one of clauses 116 to 118, wherein the syringe comprises a needle having a size of 18 to 32 Gauge (G).
120. The syringe of clause 119, wherein the needle is an 22 G needle. 121. The syringe of clause 119, wherein the needle is a 24 G needle.
122. The syringe of clause 119, wherein the needle is a 25 G needle.
123. The syringe of clause 119, wherein the needle is a 26 G needle.
124. The syringe of clause 119, wherein the needle is a 27 G needle.
125. The syringe of clause 119, wherein the needle is a 30 G needle.
126. The syringe of clause 119, wherein the needle is a 32 G needle.
127. The syringe of any one of clauses 119-126, wherein the needle is an ultra-thin walled needle.
128. A method of administering a biopharmaceutical to a subject by injection, the method comprising:
   injecting a composition according to any one of clauses 1-115 to administer a therapeutically effective dose of the biopharmaceutical to a subject in need thereof.
129. The method of clause 128, wherein the injecting is performed using a loaded syringe of any one of clauses 116-127.
130. A method of preparing an injectable pharmaceutical composition, the method comprising:
   a) providing a mixture comprising:
      a biopharmaceutical agent; and
      a polyacrylamide-based copolymer in water;
   b) removing the water from the mixture to obtain particles comprising the biopharmaceutical agent and the polyacrylamide-based copolymer;
   c) contacting a liquid carrier with the particles to form a suspension of the particles in the liquid carrier.
131. The method of clause 130, wherein in step b) the water is removed via spray drying.
132. The method of clause 130, wherein in step b) the water is removed via electrospray drying.
133. The method of clause 130, wherein in step b) the water is removed via lyophilization.
134. The method of clause 130, wherein the method further comprises a milling step after the lyophilization but prior to step c).
135. The method of any one of clauses 130 to 134, wherein the particles obtained in step b) have a mean diameter of 100 microns or less.
136. The method of any one of clauses 130 to 135, wherein step c) comprises sonicating the particles in the liquid carrier.

### 6. EXAMPLES

The Examples in this section are offered by way of illustration, and not by way of limitation. The examples can represent only some embodiments, and it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds and compositions described herein.

### General Methods

All reagent grade materials and solvents were purchased from Sigma Aldrich or Fisher and used as received. Alexa-647-NHS was purchased from Lumiprobe. Slide-A-Lyzer dialysis cassettes (2 kDa MWCO) from Thermo Fisher were used for polymer purification. BSA (A2153-50 G, CAS-No:9048-46-8) was purchased as a lyophilized powder from Sigma Aldrich. Human IgG (Cat No: 340-21, Lot: 07J4627) was purchased as a lyophilized powder from Medix Biochemica. HyPure™ Cell Culture Grade Water was purchased from Cytiva. Phosphate Buffered Saline (10010-023) was purchased from Gibco. Syringes used for injection force measurements are Fisherbrand 1 mL plastic luer lock syringes (Cat. No: 14955464). Syringes used for formulating protein suspensions with non-solvent were Thermo Scientific 5 mL luer slip plastic syringe (Cat. No: S7510-5). Needles used for injection force measurements were BD PrecisionGlide™ needles (26 G, ½ in, Ref: 305111). In-vivo protein suspension delivery was performed using BD Insulin Syringes with BD Micro-Fine™ IV Needles (28 G, 12.7 mm).

### Statistical Analysis

Injection force data is reported as means with standard deviation. For in vivo experiments, animals were cage blocked, and Mead's Resource Equation was used to identify a sample size above that additional subjects will have little impact on power. Normalized fluorescence intensity and half-life of subcutaneous absorption from in-vivo experiments are reported as means with standard error. Comparison between groups was conducted with the Tukey HSD test in JMP. Results were accepted as significant if p<0.05.

### 6.1.1. Example 1—Synthesis of Polyacrylamide-Based Copolymers

### General Methods

Polyacrylamide-based copolymer synthesis: The polyacrylamide-based copolymers may be synthesized using any convenient method. Methods which can be used or adapted for use in preparing copolymers of this disclosure include the exemplary synthetic methods described herein in Example 1.1, and those methods described by Appel et al. in PCT application No. PCT/US2021/027693, filed Apr. 16, 2021, and by Mann et al., Sci. Transl. Med. 12, eaba6676 (2020), the disclosures of which are herein incorporated by reference in their entirety.

Copolymer molecular weight characterization: Mn, Mw, and dispersity for copolymers with HEAM, DMA, MPAM, and MORPH carrier monomers are determined via SEC implementing poly(ethylene glycol) standards (American Polymer Standards Corporation) after passing through two size exclusion chromatography columns.

Mn, Mw, and dispersity for copolymers with AM are determined via SEC-MALLS after passing through a size exclusion chromatography column in a mobile phase of phosphate-buffered saline containing 300 ppm sodium azide. Detection is carried out with a Optilab T-rEX (Wyatt Technology Corporation) refractive index detector operating at 658 nm and a TREOS II light scattering detector (Wyatt Technology Corporation) operating at 659 nm. The dn/dc value for AM copolymers are assumed to be 0.185 in this media.

### Example 1.1—Synthesis of MoNi

The amphiphilic acrylamide copolymer excipient 4-acryloylmorpholine_{77%}-N-isopropylacrylamide_{23%} (MoNi 77:23, also referred to herein as “MoNi”) was prepared according to methods described by Mann et al., Sci. Transl. Med. 12, eaba6676 (2020). Briefly, MORPH (645 mg, 4.57 mmol, 41.5 eq.), NIP (105 mg, 0.93 mmol, 8.5 eq.), RAFT CTA 2-cyano-2-propyl dodecyl trithiocarbonate (2-CPDT) (38 mg, 0.11 mmol, 1 eq.), and initiator 2,2-azobis(2-methyl-propionitrile) AIBN (3.6 mg, 0.02 mmol, 0.2 eq.) were combined and diluted with N,N-dimethylformamide (DMF) to a total volume of 2.25 mL (33.3 (w/v) vinyl monomer concentration) in an 8-mL scintillation vial equipped with a PTFE septa. The reaction mixture was sparged with nitrogen gas for 10 min and then heated for 12 hours at 65° C. To remove the CTA Z terminus of the resulting polymer, AIBN (360 mg, 2.2 mmol, 20 eq.) and lauroyl peroxide (LPO) (88 mg, 0.22 mmol, 2 eq.) were added to the reaction mixture, which was then sparged with nitrogen gas for 10 min and heated for 12 hours at 90° C. CTA Z group removal was confirmed by the ratio of the refractive index to ultraviolet (310 nm) intensity in size exclusion chromatography (SEC) analysis. Resulting polymers were precipitated three times from ether and dried under vacuum overnight. Resulting composition and molecular weights were determined via ¹H NMR spectroscopy (e.g., in d₆-DMSO) and SEC with PEG standards.

### Example 1.2—Copolymer Molecular Weight Characterization by SEC

Mₙ, M_{w}, and Ð for MoNi was determined via SEC implementing PEG standards (American Polymer Standards Corporation) after passing through two SEC columns [inner diameter, 7.8 mm; Mw range, 200 to 600,000 g mol⁻¹; Resolve Mixed Bed Low divinylbenzene (DVB) (Jordi Labs)] in a mobile phase of DMF with 0.1 M LiBr at 35° C. and a flow rate of 1.0 mL min⁻¹ [Dionex UltiMate 3000 pump, degasser, and autosampler (Thermo Fisher Scientific)].

MoNi polymer was also characterized by determining the molecular weight (Mn SEC) and dispersity using an RI detector and polymethylmethacrylate standards. The running solvent was N,N-dimethylformamide (DMF) with 1 g/L LiBr (flow rate: 1 mL/min) heated to 50° C. and samples were prepared at 5 mg/mL. Separation was done through two Jordi Labs Resolve Mixed Bed Low Divinylbenzene (DVB) columns in series and data was collected by a Dionex Ultimate 3000 Variable Wavelength detector and RefractoMax521 RI detector. The RI traces were normalized and areas under the curves for the 310 nm absorbance signals were calculated with Prism 10.

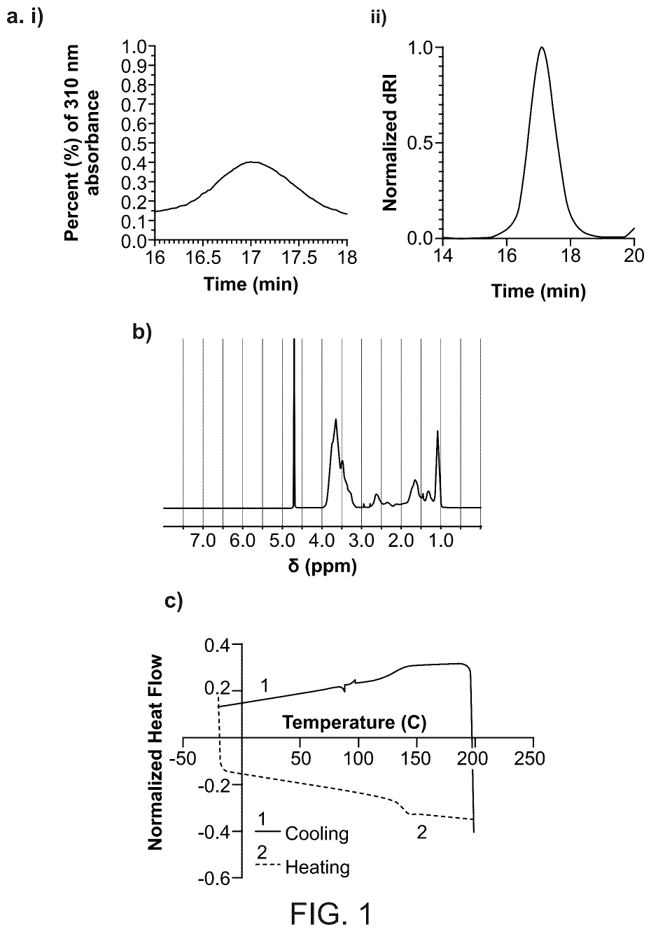

FIG. 1, panels a to c illustrate characterization of MoNi. Panel a depicts a SEC trace of MoNi. Panel b depicts a 1H NMR of MoNi. Panel c depicts a DSC of MoNi at a temperature ramp and cooling rate of 10° C./min showing a glass transition temperature between 130 and 140° C. (top line=cooling; lower line=heating).

### 6.1.2. Example 2—Preparation of Particles and Particle Suspensions

Particles and particle suspensions including a subject polyacrylamide-based copolymer and a protein agent were prepared by one of the following methods.

### Example 2.1—General Method A—Lyophilization and Ball Milling

The protein agent in water is optionally combined with a subject polyacrylamide-based copolymer and a buffer. The water is then removed by lyophilization and the resulting mixture is milled to provide particles. A liquid carrier (e.g., as described herein) is then added to the particles with mixing to provide a suspension of the particles in the liquid carrier.

### Example 2.2—General Method B—Spray Drying

The protein agent in water, or an aqueous-organic solvent mixture, is optionally combined with a subject polyacrylamide-based copolymer. The water, or an aqueous-organic mixture, is then removed by spray drying to provide particles. A liquid carrier (e.g., as described herein) is then added to the particles with mixing to provide a suspension of the particles in the liquid carrier.

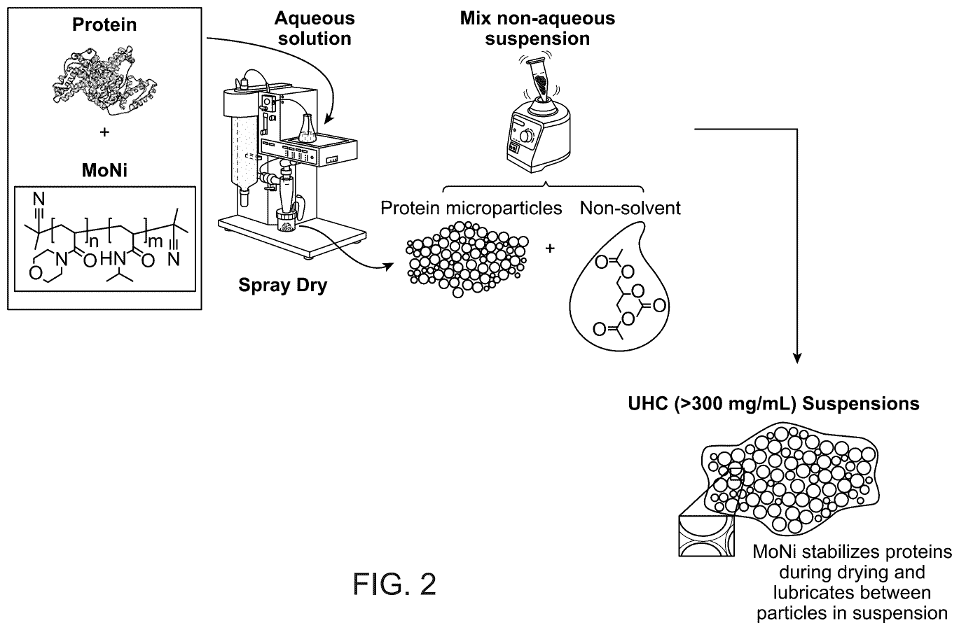

FIG. 2 illustrates a schematic of the spray drying process and formulation of example particle suspensions.

Method B1—Spray drying BSA particles: BSA feed solutions were prepared by dissolving lyophilized BSA in cell grade water at 2 wt % (20 mg/mL). BSA solutions dissolved at room temperature for one hour prior to sterile filtering using a 0.2 μm sterile filter. After sterile filtering, 7 kDa MoNi was added to the feed solution at a concentration of 0.1 wt % (1 mg/mL). Feed solutions were stored on ice prior to spray drying.

Samples were spray dried using a Buchi B-290 Mini Spray Dryer equipped with a high-performance cyclone. Samples were spray dried using an inlet temperature of 150° C. (outlet˜67) ° C., aspirator pressure of 40 mm, and a pump rate of 20% (6 mL/min). Collected particles was transferred to a 50 mL falcon tube and stored at 4° C. with desiccant.

Method B2—Spray drying BSA particles with polysorbate 80 (Tween 80): BSA feed solutions were prepared by dissolving lyophilized BSA in cell grade water at 2 wt % (20 mg/mL). BSA solutions dissolved at room temperature for one hour prior to sterile filtering using a 0.2 μm sterile filter. After sterile filtering, polysorbate 80 was added to the feed solution at a concentration of 0.0176 wt % (0.176 mg/mL). Polysorbate 80 concentration was chosen to add equal moles of MoNi and polysorbate 80 to the spray drying feed solution. Feed solutions were stored on ice prior to spray drying.

Samples were spray dried using a Buchi B-290 Mini Spray Dryer equipped with a high-performance cyclone. Samples were spray dried using an inlet temperature of 150° C. (outlet˜67) ° C., aspirator pressure of 40 mm, and a pump rate of 20% (6 mL/min). Collected particles was transferred to a 50 mL falcon tube and stored at 4° C. with desiccant.

Method B3—Spray drying human immunoglobulin G (hIgG) particles: hIgG feed solutions were prepared by dissolving lyophilized hIgG in cell grade water at 10 wt % (100 mg/mL). hIgG solutions dissolved at 4° C. for four hours prior to sterile filtering using a 0.2 μm sterile filter. Protein adherence onto sterile filters was minimized by filtering 2 wt % BSA through the sterile filter followed by five water rinses. Nanodrop was used to confirm BSA was not detectable in the filtrate. After sterile filtering the hIgG, 7 kDa MoNi was added to the feed solution at a concentration of 0.5 wt % (5 mg/mL). Feed solutions were stored on ice prior to spray drying.

Samples were spray dried using a Buchi B-290 Mini Spray Dryer equipped with a high-performance cyclone. Samples were spray dried using an inlet temperature of 130° C. (outlet˜77) ° C., aspirator pressure of 40 mm, and a pump rate of 5% (2 mL/min). Collected particles was transferred to a 50 mL falcon tube and stored at 4° C. with desiccant.

Particle characterization: Particle morphology was characterized by scanning electron microscopy (SEM). Samples were grounded to an aluminum pin stub using double-sided conductive copper tape. A 5.0 nm thick layer of pure gold was deposited onto the samples using a Leica ACE600 Vacuum system. SEM analysis was performed using the FEI Magellan 400 XHR Scanning Electron Microscope at 5.00 kV and high vacuum in field-free mode.

Particle density was measured using an AccuPyc 1330. A known sample mass of spray dried particles between 200 and 300 mg was measured into a small sample cell with a volume of 1 cm³. Particle volume was measured over 999 cycles. The known mass, as measured by analytical balance, and the average sample volume was used to calculate particle density.

Formulating suspensions: Suspensions were formulated by combining a known mass of spray dried particles with a known volume of non-solvent. Protein concentration in mg/mL was determined by assuming total volume encompassed non-solvent volume as well as spray dried particle volume. Unless otherwise specified, protein particles were assumed to have a density of 1 g/cm³.

To minimize non-solvent evaporation when preparing suspensions, spray dried particles were added to the barrel of a 6 mL luer slip syringe. The mass of spray dried particles was measured using an analytical balance. The desired volume of non-solvent or non-solvent combination was added to the syringe barrel through the syringe tip using a p200 pipette. After the syringe was capped, and the protein suspension was mixed inside the syringe using a vortex for 5 minutes or until all powder was fully dispersed. Protein suspensions were transferred from 6 mL luer slip syringes to the alternative desired syringe (1 mL luer lock syringes or insulin syringes) by back loading for force of injection experiments or animal experiments respectively.

### Example 2.3—Characterizing Flow Properties of Particle Suspensions

The flow properties of particle suspensions were characterized through rheology and injection force measurements.

Rheological characterization: Rheological testing was performed using a stress-controlled TA Instruments DHR-2 rheometer. Rheology of solid-like formulations (BSA without MoNi) was performed at 25° C. using a 20 mm diameter serrated parallel plate at a 500 μm gap. Rheology of liquid-like formulations (BSA with MoNi) was performed at 25° C. using a 40 mm cone geometry with a 50 μm gap. Frequency sweeps were performed at a strain of 1% within the linear viscoelastic regime. Flow sweeps were performed from high to low shear rates with steady state sensing.

Injection force measurements: Force of injection was quantified by measuring the force required to inject a protein particle suspension through a known needle gauge at a known flow rate using a syringe of known barrel dimensions. A force sensor was built that encompassed a load cell (FUTEK LLB300 50 lb Subminiture Load Button (Model #: LLB300, Item #: FSH03954, Serial #: 705242) attached to a syringe pump (KD Scientific Syringe Pump (Model #: LEGATO 100, Catalog #: 788100, Serial #: D103954)). An Omega Engineering Platinum Series Meter (Model #: DP8PT, Serial #: 18110196) was used to translate load cell resistance measurements to force values in Kg. The load cell was calibrated prior to measuring injection force. A lab view program records the forces measured throughout the duration of an injection experiment and displays a graph of injection force over time.

Injection force experiments were performed as follows. A 1 mL Thermo Fisher luer lock syringe with the desired needle gauge was loaded into the syringe pump. The syringe pump height was adjusted so that the load button of the force sensor was in contact with the end of the syringe plunger. The initial force was at or very close to 0 Kg. The appropriate syringe barrel dimensions as well as desired flow rate and injection volume were then selected. The syringe pump moved at the programmed rate injecting protein suspension through the attached needle. The force sensor coupled with the Omega unit measured the force required to inject the protein suspension at the desired flow rate. A lab view program recorded the forces measured throughout the duration of an injection experiment and displayed a graph of injection force over time. Force of injection was quantified by subtracting the average initial force (background) from the average plateau injection force. Injection force in Kg was converted to injection force in Newtons by multiplying by 9.81.

Exemplary Formulations: Tables 1 and 2 below provides formulations obtained by General Method A and General Method B respectively.

TABLE 1
Bovine serum albumin (BSA) injectable formulations prepared by General Method A:
Table 1: BSA Formulations - Prepared by General Method A

| | | | Polymer | | | | | | | Injectable/ |
|---|---|---|---|---|---|---|---|---|---|---|
| | BSA | Polymer | Additive | | Buffer | Organic | liquid | Protein | Solids | Needle |
| # | (mg) | Additive | Mass | Buffer | Mass | liquid | Volume | Conc. | Conc. | Guage |
| 1 | 100 | — | — | Phosphate | 23 mg | Sesame | 150 | 364 | 455 | Yes/21G |
| | | | | buffer | | oil | uL | mg/mL | mg/mL | |
| 2 | 100 | MoNi | 25 mg | Phosphate | 23 mg | Sesame | 150 | 336 | 497 | Yes/21G |
| | | | | buffer | | oil | uL | mg/mL | mg/mL | |
| 3 | 100 | — | — | Trehalose | 23 mg | Sesame | 150 | 364 | 455 | Yes/21G |
| | | | | | | oil | uL | mg/mL | mg/mL | |
| 4 | 100 | MoNi | 25 mg | Trehalose | 23 mg | Sesame | 150 | 336 | 497 | Yes/21G |
| | | | | | | oil | uL | mg/mL | mg/mL | |
| 5 | 100 | MoNi | 25 mg | Trehalose | 23 mg | Triacetin | 150 | 336 | 497 | Yes/21G |
| | | | | | | | uL | mg/mL | mg/mL | Yes/26G |
| 6 | 100 | — | — | Trehalose | 10 mg | Triacetin | 140.2 | 400 | 440 | Yes/21G |
| | | | | | | | uL | mg/mL | mg/mL | No/26G |
| 7 | 100 | MoNi | 5 mg | Trehalose | 10 mg | Triacetin | 135 | 400 | 460 | Yes/21G |
| | | | | | | | uL | mg/mL | mg/mL | Yes/26G |

Formulations 2, 4, 5 and 7 include the polyacrylamide-based copolymer MoNi. Formulations 1, 3 and 6 and comparative injectable formulations that do not include a subject polyacrylamide-based copolymer.

With reference to formulations 6 and 7, as seen in Table 1 formulation 7 which includes the subject copolymer MoNi as a polymer additive in the particles and a total solids content of 460 mg/mL is injectable through both a 21 G and a 26 G needle, whereas formulation 6 which has a lower total solids content and does not include any polymer additive is injectable through a 21 G needle, but not a 26 G needle.

TABLE 2
Bovine serum albumin (BSA) injectable formulations prepared by General Method B:
Table 2: BSA Formulations - Prepared by General Method B

| | | | Polymer | | | | | | | Injectable/ |
|---|---|---|---|---|---|---|---|---|---|---|
| | BSA | Polymer | Additive | | Buffer | Organic | Liquid | Protein | Solids | Needle |
| # | (mg) | Additive | Mass | Buffer | Mass | Liquid | Volume | Conc. | Conc. | Guage |
| 8 | 100 | — | — | none | 0 mg | triacetin | 132 | 430 | 430 | Yes/27G |
| | | | | | | | uL | mg/mL | mg/mL | No/30G |
| 9 | 100 | MoNi | 5 mg | none | 0 mg | triacetin | 121 | 430 | 442 | Yes/27G |
| | | | | | | | uL | mg/mL | mg/mL | Yes/30G |
| 10 | 100 | — | — | none | 0 mg | triacetin | 116 | 460 | 460 | No/27G |
| | | | | | | | uL | mg/mL | mg/mL | |
| 11 | 100 | MoNi | 5 mg | none | 0 mg | triacetin | 111 | 460 | 486 | Yes/27G |
| | | | | | | | uL | mg/mL | mg/mL | |

Formulations 9 and 11 include the polyacrylamide-based copolymer MoNi. Formulations 8 and 10 and comparative injectable formulations that do not include a subject polyacrylamide-based copolymer.

With reference to formulations 8 and 9, as seen in Table 2 formulation 9 which includes the subject copolymer MoNi as a polymer additive and a total solids content of 442 mg/mL is injectable through both a 27 G and a 30 G needle, whereas formulation 8 which has a lower total solids content and does not include any polymer additive is injectable through a 27 G needle, but not a 30 G needle. Similarly, with reference to formulations 10 and 11, as seen in Table 2 formulation 11 which includes the subject copolymer MoNi as a polymer additive and a total solids content of 486 mg/mL is injectable through a 27 G needle, whereas formulation 10 which has a lower total solids content and does not include any polymer additive is not injectable through a 27 G needle.

These results demonstrate that addition of a small amount of a subject copolymer (e.g., MoNi) to a protein formulation (e.g., BSA) provide improved injection properties despite higher solids content.

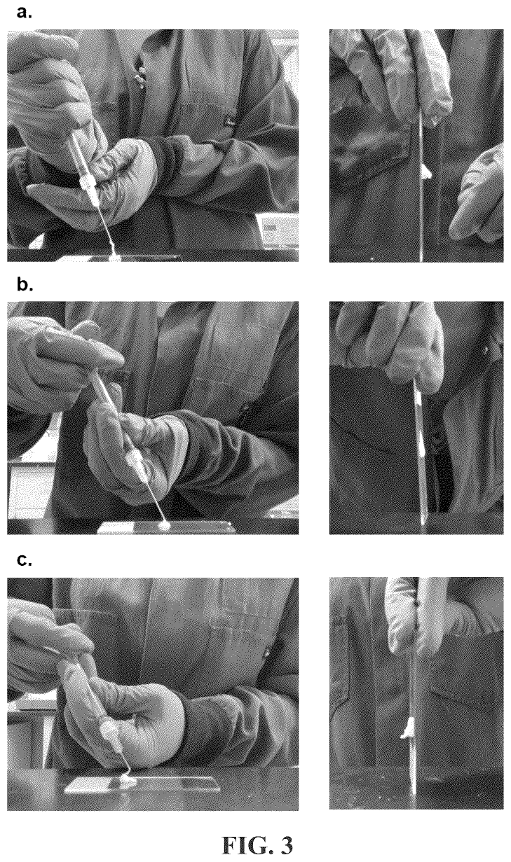

FIG. 3, panels a-c illustrate injection behavior of various formulations. Panel a illustrates the injection through a 21 G needle and depot forming behavior of formulation 4 (336 mg/mL BSA in sesame oil including the copolymer MoNi and trehalose, total solids of 497 mg/mL). Panel b illustrates the injection through a 21 G needle and lack of depot forming behavior of formulation 3 (364 mg/mL BSA in sesame oil including trehalose, but no copolymer, total solids of 455 mg/mL). Panel c illustrates the injection though a 26 G needle and depot forming behavior of formulation 7 (400 mg/mL BSA in triacetin including the copolymer MoNi and trehalose, total solids of 460 mg/mL).

As illustrated by FIG. 1, panels a-c, the addition of the subject copolymer MoNi modifies injection and rheological properties of the formulations.

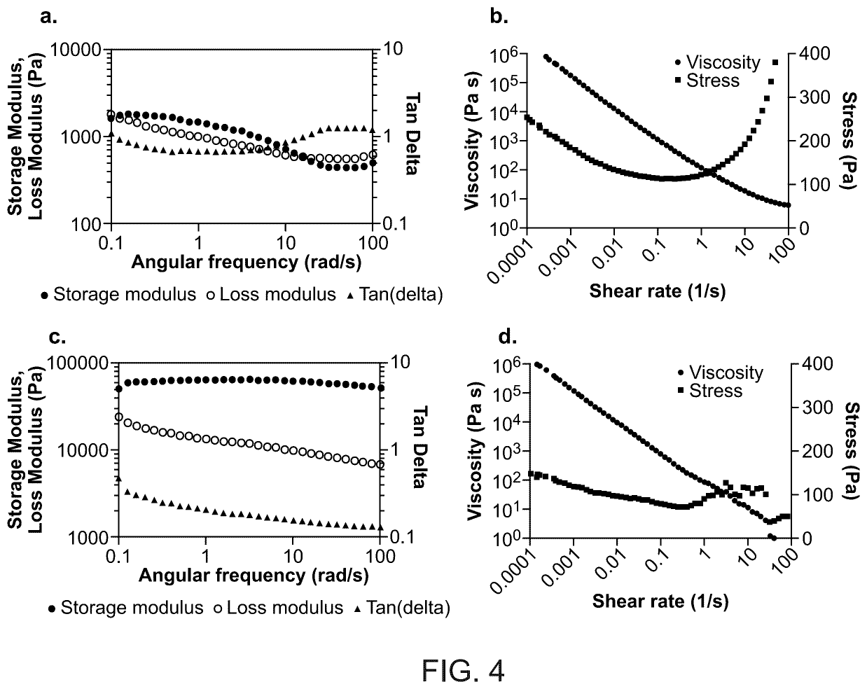

FIG. 4, panels a-d illustrate angular frequency sweep and flow sweep of formulations 6 and 7. Panel a illustrates the angular frequency sweep for formulation 7 (400 mg/mL BSA in triacetin with MoNi). Panel b illustrates the flow sweep for formulation 7 (400 mg/mL BSA in triacetin with MoNi). Panel c illustrates the angular frequency sweep for formulation 6 (400 mg/mL BSA in triacetin without copolymer). Panel d illustrates the flow sweep for formulation 6 (400 mg/mL BSA in triacetin without copolymer).

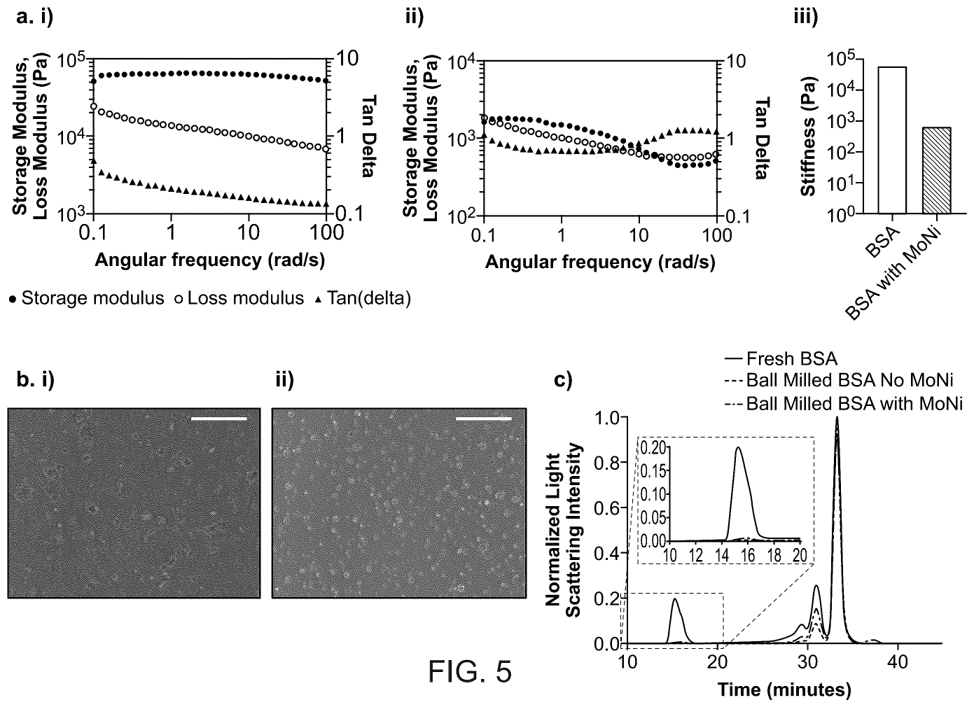

FIG. 5, panels a-c illustrate rheological and stability characterization of ball milled BSA particles. Panel a illustrates angular frequency sweep of i) 400 mg/mL BSA particles resuspended in triacetin; and ii) 400 mg/mL BSA particles containing 5 wt % MoNi resuspended in triacetin. Panel a. iii) Comparative storage modulus of suspensions at 10 rad/sec. Panel b illustrates microscope images of BSA with 5 wt % MoNi particles formed by i) 15 minutes of ball milling or ii) spray drying. Particles are resuspended in sesame oil for improved imaging. Panel c depicts a SEC trace of fresh BSA control, 15 minute ball milled BSA without MoNi, and 15 minute ball milled BSA with 5 wt % MoNi. PBS with sodium azide is used as an eluent.

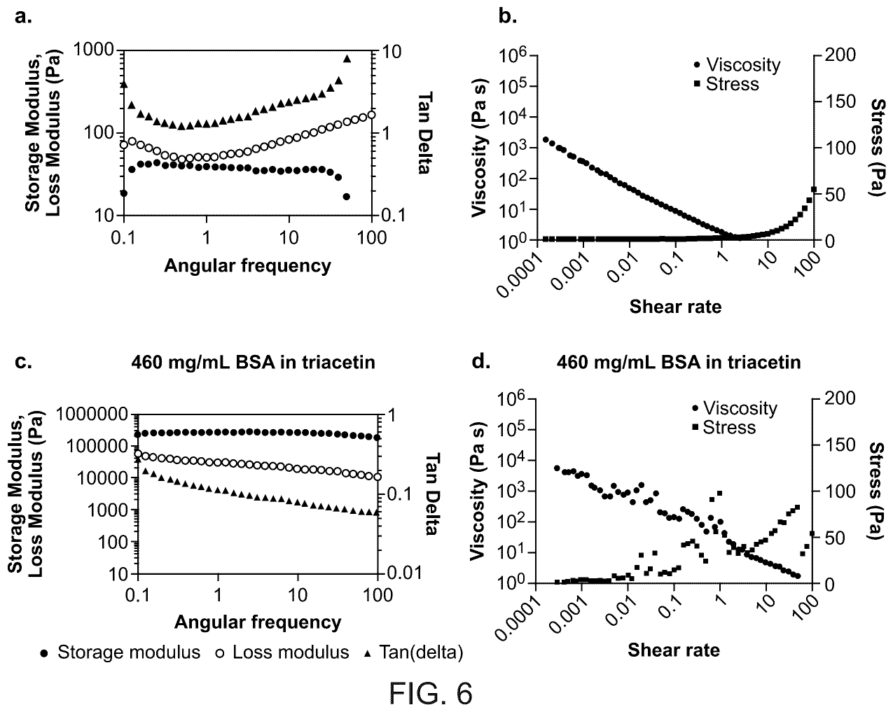

FIG. 6, panels a-d illustrate angular frequency sweep and flow sweep of formulations 10 and 11. Panel a illustrates the angular frequency sweep for formulation 11 (460 mg/mL BSA in triacetin with MoNi). Panel b illustrates the flow sweep for formulation 11 (460 mg/mL BSA in triacetin with MoNi). Panel c illustrates the angular frequency sweep for formulation 10 (460 mg/mL BSA in triacetin without copolymer). Panel d illustrates the flow sweep for formulation 10 (460 mg/mL BSA in triacetin without copolymer).

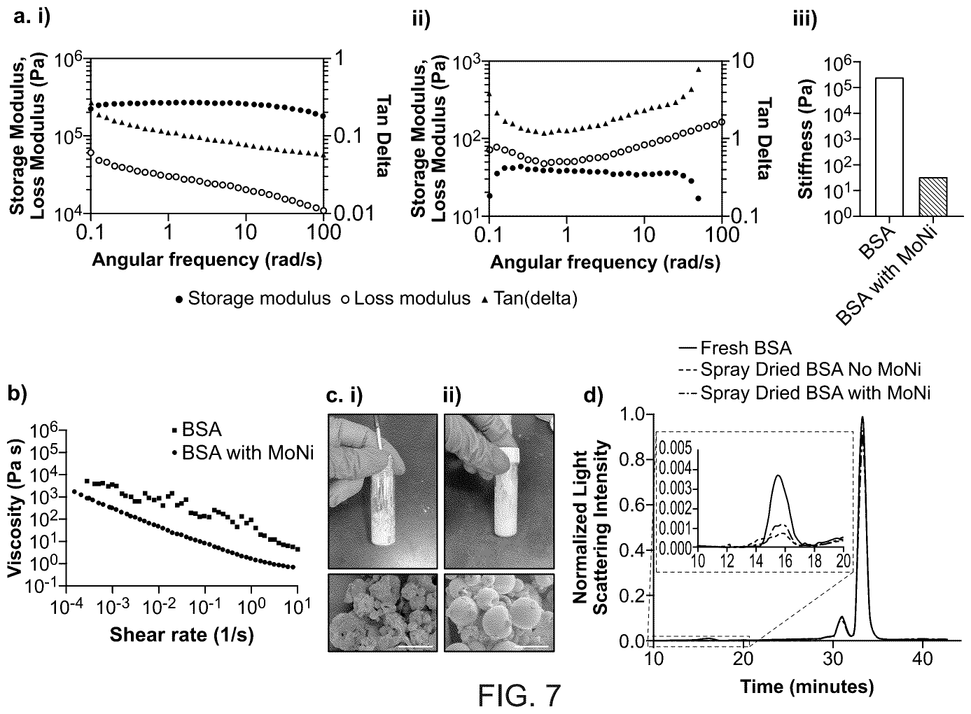

FIG. 7, panels a-d illustrate rheological and stability characterization of spray dried particles. Panel a illustrates angular frequency sweep of i) 460 mg/mL BSA particles resuspended in triacetin; and ii) 460 mg/mL BSA particles containing 5 wt % MoNi resuspended in triacetin. Panel a. iii) Comparative storage modulus of suspensions at 10 rad/sec. Panel b illustrates flow sweep of 460 mg/mL BSA particles resuspended in triacetin and 460 mg/ml BSA particles containing 5 wt % MoNi resuspended in triacetin. Panel c depicts a SEM image of particles (scale bar 5 μm) and image of resultant suspension in triacetin at 460 mg/mL for particles formulated i) without and ii) with 5 wt % MoNi. Panel d depicts a SEC trace of fresh BSA control, spray dried BSA without MoNi, and spray dried BSA with 5 wt % MoNi. PBS with sodium azide is used as an eluent.

Rheological characterization of the protein particle suspensions in triacetin demonstrates that the addition of 5 wt % MoNi to the protein particles improves the flow properties of the protein suspension. As shown in FIG. 7, protein suspensions were prepared at 460 mg/mL BSA using particles with and without 5 wt % MoNi. Without the addition of MoNi, protein suspensions have the consistency of a thick solid paste and do not flow; however, with MoNi, protein suspensions instead flow like a liquid. Angular frequency sweeps demonstrate that the addition of MoNi reduced the storage and loss moduli of suspensions by 2 orders of magnitude. Additionally, the tan delta of the suspension with MoNi exceeded 1 indicating liquid-like behavior, while the suspensions without MoNi had tan deltas below 1 implying solid-like behavior (FIG. 7, panel a). Flow sweeps further demonstrate that protein suspensions are shear thinning, and MoNi results in a decrease in suspension viscosity (FIG. 7, panel b). SEM images of spray dried microparticles illustrate that while microparticles spray dried with and without MoNi are similar in average diameter (5-10 μm), particles with MoNi have a smooth, spherical morphology while particles without MoNi show a wrinkled surface morphology (FIG. 7, panel c). This difference in spray dried particle morphology in the presence of the MoNi surfactant may contribute to decreased yield stress of the protein suspension improving flow properties. Beyond flow, MoNi also stabilizes BSA through the high-temperature spray drying process. SEC characterization of fresh BSA as well as BSA spray dried with and without MoNi shows that spray drying BSA with MoNi results in a higher monomer peak fraction and a smaller high molecular weight aggregate peak (FIG. 7, panel d). These improvements in BSA stability and suspension flow with MoNi were also observed when protein microparticles were instead formed by lyophilization followed by ball milling (FIG. 5, panels a-c).

### 6.1.3. Example 3—Characterization of Protein Stability in Injectable Formulations Formed by Method A

To investigate the protein stability in the subject injectable formulations, the formulations in Table 3 were prepared by General Method A.

TABLE 3
Milled Lyophilate Compositions for Stability Testing

| Formulation | BSA | MoNi | | Buffer | | Milling |
|---|---|---|---|---|---|---|
| Label | Mass | Mass | Buffer | Mass | Lyophilized? | Time |
| BSA (without | 100 mg | 0 mg | None | 0 mg | no | 0 min |
| lyophilization) | | | | | | |
| 20BSA.1/2T.0 min | 100 mg | 0 mg | Trehalose | 10 mg | yes | 0 min |
| 20BSA.MoNi.1/2T.0 min | 100 mg | 5 mg | Trehalose | 10 mg | yes | 0 min |
| 20BSA.T.0 min | 100 mg | 0 mg | Trehalose | 20 mg | yes | 0 min |
| 20BSA.MoNi.T.0 min | 100 mg | 5 mg | Trehalose | 20 mg | yes | 0 min |
| 20BSA.1/2T.15 min | 100 mg | 0 mg | Trehalose | 10 mg | yes | 15 min |
| 20BSA.MoNi.1/2T.15 min | 100 mg | 5 mg | Trehalose | 10 mg | yes | 15 min |
| 20BSA.T.15 min | 100 mg | 0 mg | Trehalose | 20 mg | yes | 15 min |
| 20BSA.MoNi.T.15 min | 100 mg | 5 mg | Trehalose | 20 mg | yes | 15 min |

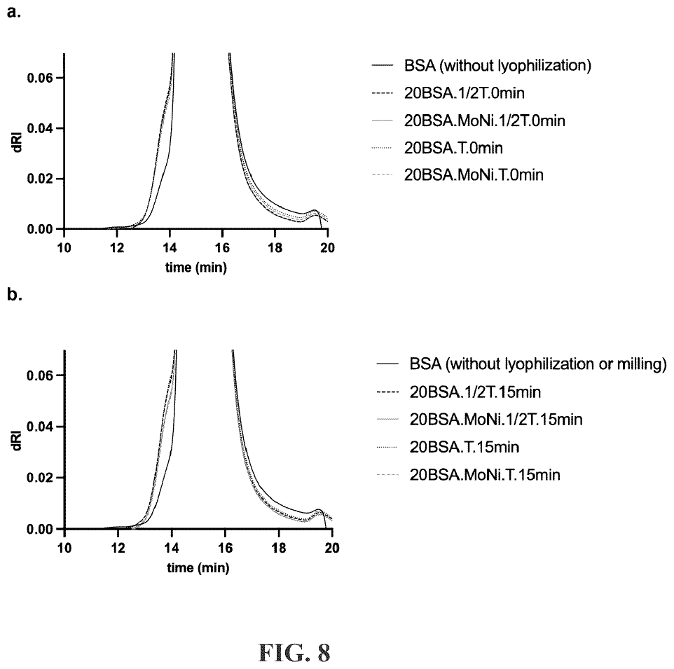

FIG. 8, panels a-b illustrate the impact of Trehalose and copolymer MoNi on BSA stability after lyophilization (panel a) and milling (panel b). Panel a demonstrates that trehalose and MoNi content during lyophilization show little impact on high molecular weight shoulder. Panel b further demonstrates that irrespective of trehalose content, MoNi reduces size of high molecular weight shoulder following 15 minutes of ball milling.

### 6.1.4. Example 4—Characterization of Protein Stability Following Spray Drying, and Comparison to Ball Milling

Protein stability before and after spray drying was characterized by SEC. Protein suspension stability was characterized by SEC as well as by comparative injection force.

### Example 4.1—Characterization of Protein Stability

SEC characterization: The SEC trace of protein samples was determined with the ASTRA software package (Wyatt Technology Corporation) after passing a 5 mg/mL protein sample through a size-exclusion chromatography column (Superose 6 Increase 10/300 GL) in a mobile phase of PBS with sodium azide at 25° C. and a flow rate of 0.5 mL/min. Detection consisted of a Optilab T-rEX (Wyatt Technology Corporation) refractive index detector operating at 658 nm and a TREOS II light scattering detector (Wyatt Technology Corporation) operating at 659 nm. A dn/dc value of 0.185 was used for BSA and IgG samples. To directly compare the stability of spray dried protein samples, SEC traces were normalized to the height of the monomer peak.

Methodology for stressed aging: BSA protein suspensions were prepared at 460 mg/mL in triacetin using spray dried BSA particles with and without 5 wt % MoNi. A BSA aqueous control was prepared by dissolving fresh, lyophilized BSA at 20 mg/mL in PBS. All samples were stored in parafilmed 8 mL scintillation vials. A 500 ml beaker of water was heated to 60° C. using a temperature controlled hot plate. The sample files were submerged in the 60° C. water bath so that the protein sample volume was fully beneath the water line. Samples were heated at 60° C. for 30 minutes to encourage protein degradation. Following stressed aging, samples were redissolved in PBS at 5 mg/mL, and protein stability was assessed through SEC.

Protein suspension storage and injection force measurements: BSA protein suspensions with 5 wt % MoNi were prepared at 460 mg/mL in triacetin. Protein suspensions were loaded into a 1 mL Thermo Fisher luer slip syringe and capped with a BD 26 G ½ inch needle wrapped with parafilm to limit solvent evaporation. Injection force for the 460 mg/mL protein suspension was measured on day 0 and again on day 35. The syringe was stored horizontally at 23° C.

### Example 4.2—Comparison of Spray Dried Vs Ball Milled Particles

Exemplary BSA protein particle compositions prepared via spray drying or ball milling were assessed for protein stability using size exclusion chromatography (SEC).

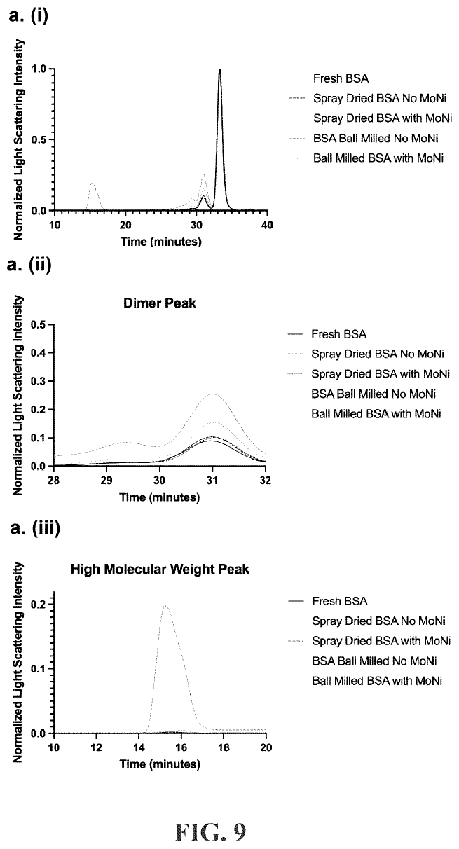

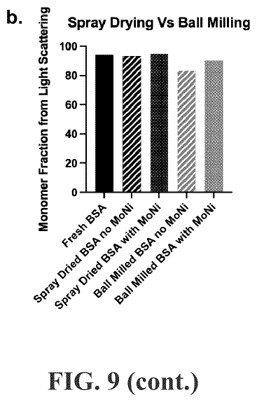

FIG. 9, panels a(i)-a(iii) and b show the results of assessment of exemplary particle compositions for protein stability using SEC. SEC column with improved resolution allows for visualization of BSA monomer and dimer peak. Panel a(i): Full SEC traces of fresh, spray dried, and ball milled BSA. Panel a(ii): Dimer peak of fresh, spray dried, and ball milled BSA. Panel a(iii): High molecular weight peak of fresh, spray-dried particles with a 100:5 weight ratio of BSA, and ball milled BSA. Panel b: BSA monomer fraction after spray drying and ball milling.

Fresh BSA as well as spray dried BSA with and without MoNi show nearly identical SEC traces indicating that BSA is stable through the spray drying process. BSA spray dried with MoNi has a slightly higher monomer fraction suggesting improved stability. Ball milled BSA shows a higher dimer peak than spray dried BSA, and the dimer peak of ball milled BSA with MoNi is smaller than the dimer peak of ball milled BSA alone. These findings suggest that spray drying is a gentler process of forming particles than ball milling, but that MoNi is a useful protectant through the ball milling process. SEC traces were obtained using a Superose 6 column. PBS with sodium azide is used as an eluent.

### 6.1.5. Example 5—Injectability of Suspension Formulations Prepared by General Method B

To investigate the injectability of the subject formulations, injection force measurements were taken for formulation 11 (containing spray-dried particles with a 100:5 ratio of BSA to copolymer MoNi at a total protein concentration of 460 mg/mL in triacetin).

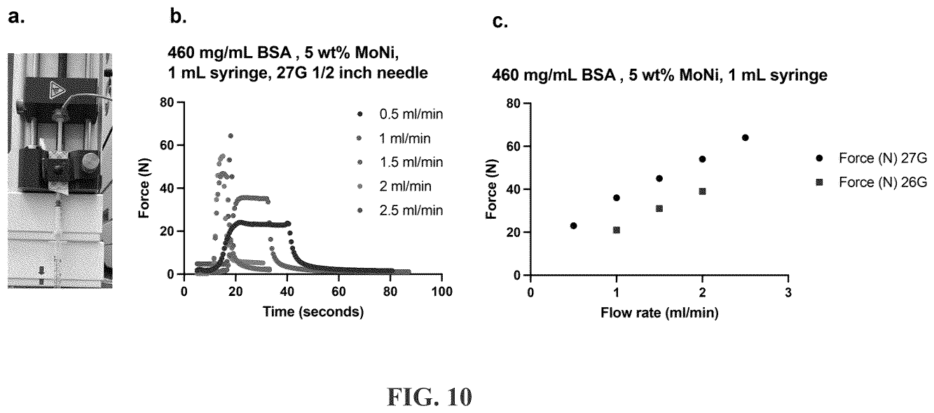

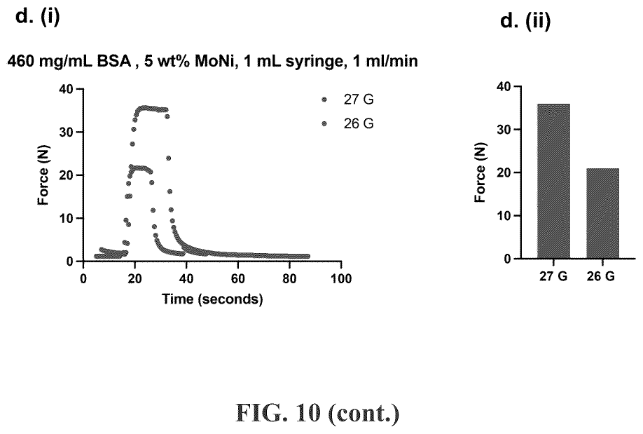

FIG. 10, panels a-c and d(i)-d(ii). Panel a: A force sensor connected to a syringe pump was used to quantify the force of injection through standard gauge needles using a 1 mL syringe. Panel b: Force of injection curves illustrate injection force needed to inject compositions comprising 460 mg/mL BSA and 23 mg/mL MoNi compositions (i.e., 5 wt % of the particle solids) through 27 G ½ inch needles at varied flow rates. Concentration of BSA assumes a particle density of 1 g/cm\hat{ }3. See Example 11 below with respect to BSA particle density. Concentration of BSA estimated to be closer to 519.5 mg/mL. Panel c: Force of injection is linear with flow rate and scales with needle gauge. Panel d(i): Force of injection curves illustrate injection force needed to inject compositions comprising 460 mg/mL BSA and 23 mg/mL MoNi (i.e., 5 wt % of the suspended particle solids) through 27 G ½ inch needles and 26 G ½ inch needles at 1 mL/min. Panel d(ii): Injection force decreases with increased needle gauge. Injection force comparisons to BSA formulations not including MoNi are not included because formulations not including MoNi are not injectable through 26 or 27 G needles.

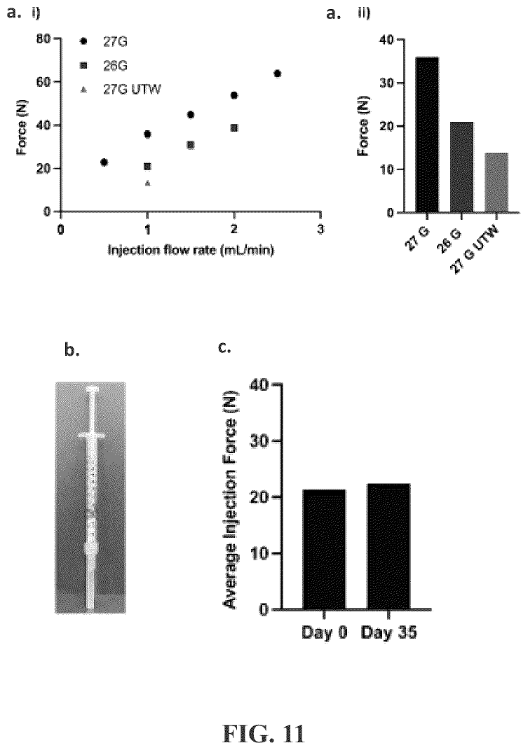

FIG. 11, panels a-c. (Panel a) i) Further illustrates that force of injection is linear with flow rate, and ii) scales with needle gauge. (Panel b) Illustration of 460 mg/mL BSA with 5% MoNi in triacetin following vertical storage in a syringe for 120 hours. (panel c) Injection force of 460 mg/mL BSA with 5% MoNi in triacetin at day 0 and day 35 using a 26 G ½ inch needle and a flow rate of 1 mL/min.

For therapeutic applications protein suspension should be injectable through clinically relevant needle gauges with clinically relevant injection forces. To assess the injectability of suspensions of BSA with MoNi in triacetin, the injection force required to inject a 460 mg/mL BSA suspension through a 27 gauge ½ inch needle at a given flow rate was quantified using a force sensor attached to a syringe pump (FIG. 10, panel a). Injection force increases linearly with flow rate of injection and decreases with the use of either smaller gauge needles (larger inner diameter) or thin-walled needles (FIG. 10, panels b-d, and FIG. 11, panel a). Using a clinically relevant 27 gauge UTW needle and a flow rate of 1 mL/min, the 460 mg/mL BSA suspension had an injection force of only 14 N (FIG. 11, panel a)—well within the injection force range relevant to pen autoinjectors (see, e.g., Hill, R. L. et al. Med Devices-Evid Res 9, 257-266 (2016)). Long-term storage experiments further demonstrate suspensions show minimal particle settling in the triacetin non-solvent (FIG. 11, panel b) and comparable injection forces (21.4 N vs. 22.4 N) following 35 days of storage (FIG. 11, panel c).

### 6.1.6. Example 6—Assessment of Stability Following Storage and Stressed Aging

Exemplary BSA protein particle compositions were assessed for storage stability.

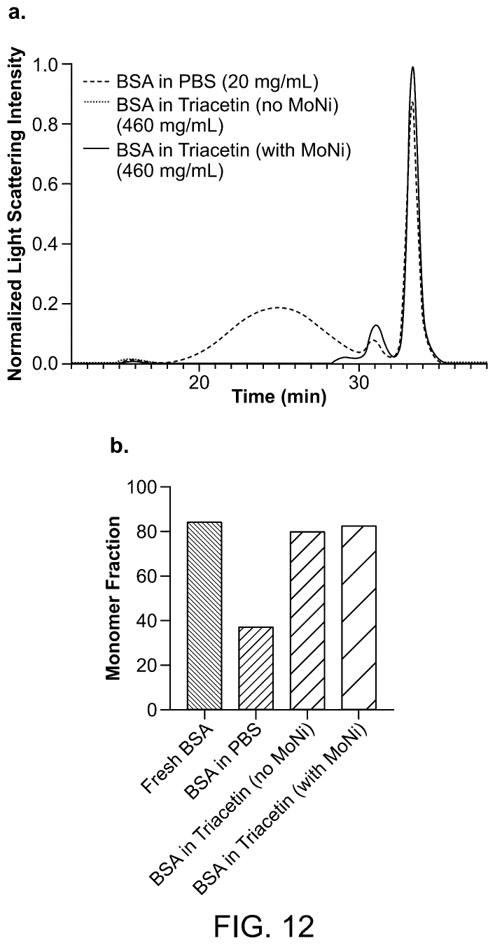

FIG. 12, panels a-b. (Panel a) Further illustrates a SEC trace of BSA at 20 mg/mL in PBS, spray dried BSA without MoNi at 460 mg/mL in triacetin, and spray dried BSA with 5 wt % MoNi at 460 mg/mL in triacetin following stressed aging for 30 minutes at 60° C. (Panel b) Corresponding monomer fraction for each formulation following stressed aging.

As set out above, comparative stability of BSA in solution and BSA in a triacetin suspension was assessed via a stressed aging assay. BSA protein suspensions were prepared at 460 mg/mL in triacetin using spray dried BSA particles with and without 5 wt % MoNi. A BSA aqueous control was prepared by dissolving fresh, lyophilized BSA at 20 mg/mL in PBS. Samples were heated at 60° C. for 30 minutes, and their stability was then assessed by SEC (FIG. 12, panel a). While BSA dissolved in PBS showed significant aggregation and a reduced monomer peak fraction (37%), solid BSA particles dispersed in non-solvent remain stable (monomer peak fraction of greater than 80%) through high temperature conditions (FIG. 12, panel b). To further compare the stability of BSA suspensions with and without MoNi, 460 mg/mL suspensions in triacetin were stored for 48 and 120 h at 4° C., 25° C., and 37° C. and stability was assessed by SEC. Suspensions with MoNi showed smaller high molecular weight aggregate peaks than suspensions with BSA alone (see Example 7).

### 6.1.7. Example 7—Stability of Protein During Storage in Suspension Form Over Time

To investigate the protein stability in the injectable formulations during storage over time, formulation 11 (containing spray-dried particles with BSA and copolymer MoNi at a total protein concentration of 460 mg/mL in triacetin) was stored at various temperatures (4° C., 25° C. and 37° C.), and samples were evaluated after 48 hours and 120 hours.

BSA protein suspensions with 5 wt % MoNi were prepared at 460 mg/mL in triacetin. Protein suspensions were loaded into a 1 mL Thermo Fisher luer slip syringe and capped with a BD 26 G ½ inch needle wrapped with parafilm to limit solvent evaporation. Injection force for the 460 mg/mL protein suspension was measured on day 0 and again on day 35. The syringe was stored horizontally at 23° C.

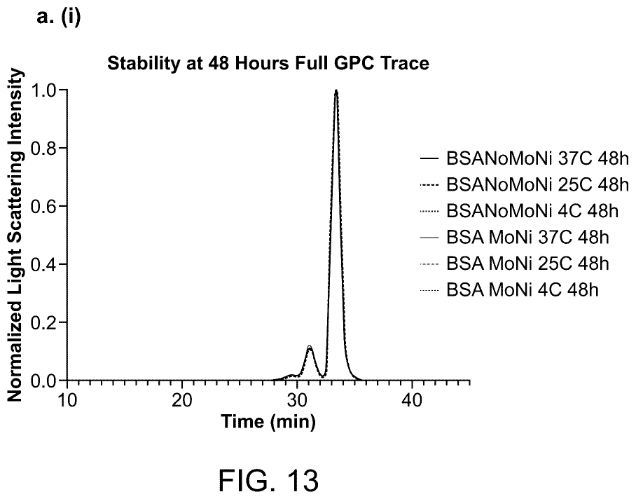

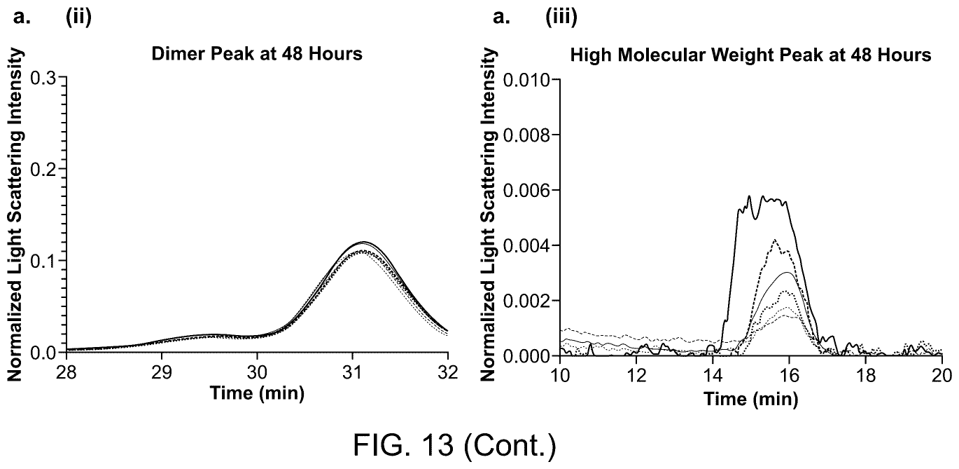

FIG. 13, panels a(i)-a(iii). 460 mg/mL BSA formulations in triacetin with and without 5 wt % MoNi were stored for 48 hours at 4 C, 25 C, and 37 C to assess stability. Panel a(i): Full SEC traces of formulations after 48 hours. Panel a(ii): Dimer peak of all formulations after 48 hours. Panel a(iii): High molecular weight peak of all formulations after 48 hours. Generally, all formulations show good stability after 48 hours with the majority of light scattering signal coming from the monomer peak. Samples containing MoNi on average show smaller dimer and high molecular weight peaks than samples without MoNi suggesting improved storage stability. SEC traces were obtained using a Superose 6 column. PBS with sodium azide is used as an eluent.

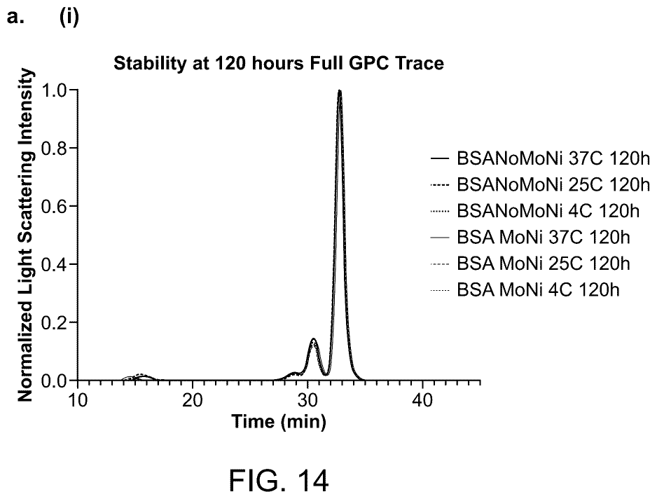

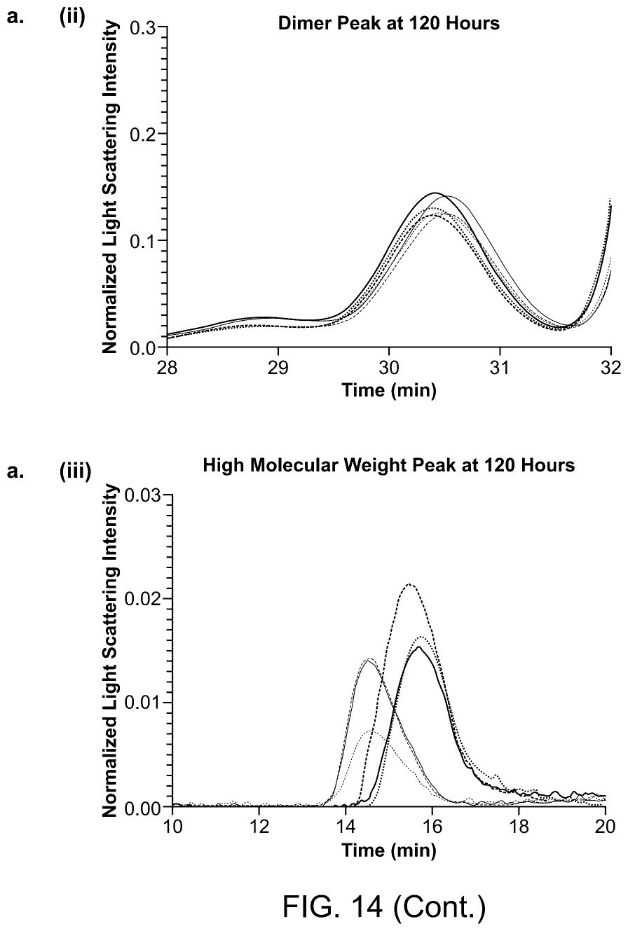

FIG. 14, panels a(i)-a(iii). 460 mg/mL BSA formulations in triacetin with and without 5 wt % MoNi were stored for 120 hours at 4 C, 25 C, and 37 C to assess stability. Panel a(i): Full SEC traces of formulations after 120 hours. Panel a(ii): Dimer peak of all formulations after 120 hours. Panel a(iii): High molecular weight peak of all formulations after 120 hours. BSA formulations containing MoNi stored for 120 hours in a syringe show no particle settling. Generally, all formulations show good stability after 120 hours with the majority of light scattering signal coming from the monomer peak. Samples containing MoNi on average show smaller high molecular weight peaks than samples without MoNi suggesting improved storage stability. SEC traces were obtained using a Superose 6 column. PBS with sodium azide is used as an eluent.

### 6.1.8. Example 8—Liquid Carrier Studies

To investigate the liquid carrier component of the subject compositions, a number of solvents were tested for their suspension performance characteristics. Compositions were prepared by dispersing BSA/MoNi (5%) microparticles in each liquid carrier at 460 mg/mL. Criteria for selecting a suitable liquid carrier include the following:
i) Viscosity of less than 25 cp at 25° C.;
ii) whether or not the particular solvent had been used previously in FDA approved parenteral drug products at exposure levels above what would be targeted in the present disclosure;
iii) dispersion time upon administration i.e., at a rate that ensures the formulation does not form a depot;
iv) compatibility with contact surfaces, i.e., does not dissolve silicon glass treatments;
v) poor protein solubilization, i.e., to ensure it is a non-solvent for the particles;
vi) having a density that closely matches that of the particle density, i.e., to maximize suspension stability; and
vii) low volatility, noting that volatility concerns can be addressed by combining higher volatility solvents and lower volatility solvents, and by using airtight storage conditions.

Table 4 illustrates various liquid carriers tested along with an evaluation of their suspension performance.

### Suspension Performance Key:

| | |
|---|---|
| −− | Very bad |
| − | bad |
| +/− | neutral |
| + | good |
| ++ | Very good |

TABLE 4
Parameters of Liquid Carriers and their Suspension Performance

| | | | Vapor | | |
|---|---|---|---|---|---|
| | Density | Viscosity | pressure | Suspension | |
| Media | (g/cm\hat{ }3) | (@25 C.) | (Pa) | Performance | Additional comments |
| N-methyl- | 1.03 | 1.89 | 45 | +/− | Forms very low viscosity |
| 2- | | | | | solutions upon initial dispersion. |
| pyrrolidone | | | | | Solutions seem reasonably stable |
| (NMP) | | | | | if stored in parafilmed |
| | | | | | conditions; however solutions |
| | | | | | show coloration gradations that |
| | | | | | may be indicative of protein |
| | | | | | clumping or settling. When left |
| | | | | | in open air on a glass slide, NMP |
| | | | | | will evaporate, and protein will |
| | | | | | aggregate. |
| Dimethyl | 1.09 | 2.0 | 80 | −− | Initially forms a low viscosity |
| sulfoxide | | | | | solution upon dispersion, but |
| (DMSO) | | | | | quickly hardens into a flaky |
| | | | | | paste (within 1 hour when sealed |
| | | | | | in a glass container with |
| | | | | | parafilm). |
| Propylene | 1.03 | 48.6 | 11 | −− | Initially forms a high viscosity |
| glycol (PG) | | | | | paste upon dispersion, but |
| | | | | | quickly hardens into a sticky |
| | | | | | rock (within a couple hours |
| | | | | | when sealed in a glass container |
| | | | | | with parafilm). |
| Benzyl | 1.04 | 5.4 | 12 | + | Forms very low viscosity |
| Alcohol | | | | | solutions upon initial dispersion. |
| (BA) | | | | | Over several days these |
| | | | | | solutions become drier and |
| | | | | | flakier. It is unclear if this is due |
| | | | | | to evaporation or protein |
| | | | | | solvation. |
| Ethyl | 0.79 | 1.07 | 6000 | −− | Clumps immediately, cannot |
| alcohol | | | | | fully disperse powder. |
| Triacetin | 1.16 | 23 | 0.267 | ++ | A very good non-solvent. Shows |
| | | | | | good shelf stability and |
| | | | | | consistent injection forces |
| | | | | | following storage. Effective in |
| | | | | | initial in-vivo experiments. It is |
| | | | | | possible to reduce the viscosity |
| | | | | | by blending with other non- |
| | | | | | solvents. |
| Benzyl | 1.11 | 8.29 | 0.03 | + | Forms a low viscosity solution |
| benzoate | | | | | upon initial dispersion. |
| | | | | | Consistency is like that of |
| | | | | | triacetin. |
| Dimethyl | 0.94 | 0.92 | 173 | + | Forms very low viscosity |
| acetamide | | | | | solutions upon initial dispersion. |
| (DMAc) | | | | | Solutions are stable for at least a |
| | | | | | week when stored in glass |
| | | | | | containers with parafilm, and we |
| | | | | | see no coloration gradients. |
| | | | | | Concerns include DMAc's high |
| | | | | | vapor pressure and ability to |
| | | | | | cause irritation when delivered |
| | | | | | subcutaneously. |
| Safflower | 0.921 | 30 | N/A | +/− | Forms medium viscosity |
| Oil | | | | | solutions upon dispersion. |
| | | | | | Solutions are stable. Due to |
| | | | | | density and polarity differences, |
| | | | | | it is hard to combine oils with |
| | | | | | other non-solvents. |
| Sesame oil | 0.92 | 55 | N/A | +/− | Forms medium viscosity |
| | | | | | solutions upon dispersion. |
| | | | | | Solutions are stable. Due to |
| | | | | | density and polarity differences, |
| | | | | | it is hard to combine oils with |
| | | | | | other non-solvents. |
| DMAc:Triacetin | 0.94-1.16 | 0.92-23 | 0.267-173 | ++ | Significantly lower viscosity |
| blend (many | | | | | than triacetin alone - much |
| ratios v/v) | | | | | closer to DMAC alone. Solvents |
| | | | | | seem well miscible. Lower |
| | | | | | viscosity leads to significantly |
| | | | | | lower injection forces. |
| BA:PG | 1.03-1.04 | 16 | 11-12 | −− | Reducing the viscosity by |
| blend | | | | | adding BA to PG was not |
| (50:50 v/v) | | | | | sufficient to improve PG |
| | | | | | suspensions. Initially forms a |
| | | | | | medium viscosity paste upon |
| | | | | | dispersion, but quickly hardens |
| | | | | | into a sticky rock. |
| Triacetin:BA | 1.04-1.16 | 10, 7.5 | 0.267-12 | + | Initially reduces viscosity of |
| (80:20, | | | | | triacetin suspensions. However, |
| 50:50 v/v) | | | | | after storage for several days it is |
| | | | | | unclear if suspensions with BA |
| | | | | | are superior. |
| Triacetin:Benzyl | 1.16-1.11 | 8.3-23 | 0.03-0.267 | +, maybe ++ | Has only been tried with low |
| benzoate | | | | | concentrations of protein. This |
| (80:20 v/v) | | | | | combination should be |
| | | | | | investigated further. |
| Triacetin: | 0.94-1.16 | 0.92-23 | 0.267-173 | + | Significantly lower viscosity |
| DMAc:BA | | | | | than triacetin alone. It is unclear |
| (2:3:1) | | | | | if adding BA is any better than |
| | | | | | Triacetin and DMAc alone. |

### Media Density Studies

As outlined above, one of the criteria for selecting a suitable liquid carrier is that the density of the liquid carrier matches as closely as possible to the particle density in the composition, so as to maximize suspension stability.

Particle settling can be evaluated experimentally by centrifuge studies. In such studies, BSA is added at low concentrations (50 mg/mL) to various non-solvent mixtures. The resulting mixtures are then centrifuged for equal time, after which the volume of BSA powder than has settled to the base of each formulation is compared. When the non-solvent mixture and the BSA powder are more similar in density, reduced settling is observed.

FIG. 15 shows the results of a centrifuge study to evaluate suspension stability in the following media from left to right: triacetin alone; triacetin with 20% benzyl benzoate; triacetin with 20% benzyl alcohol; and triacetin with 20% safflower oil.

### Media Viscosity Studies

As outlined above, one of the criteria for selecting a suitable liquid carrier is that the liquid carrier has a low viscosity (i.e., viscosity of less than 25 cp at 25° C.).

To improve suspension injectability, it may be advantageous to reduce liquid carrier viscosity by using a combination of solvents in the liquid carrier. By adding a low viscosity non-solvent to a higher viscosity non-solvent, we observed a reduction in viscosity. This viscosity reduction is non-linear, so even by adding a small amount of low-viscosity non-solvent, we may see improvements in viscosity and suspension injectability.

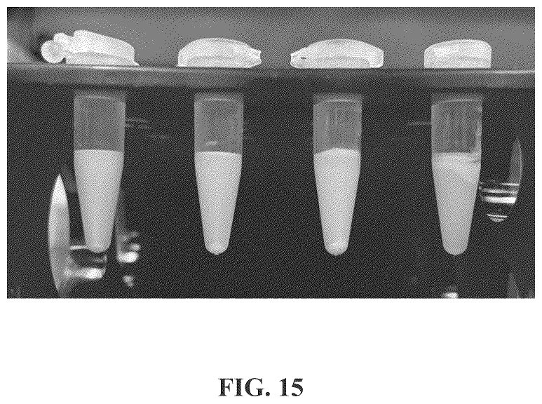

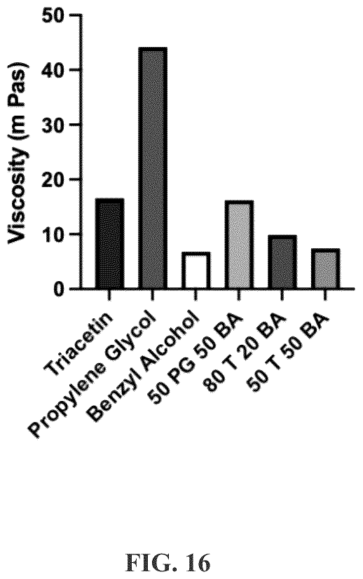

FIG. 16 illustrates viscometer measurements for various liquid carriers. This graph illustrates how the viscosity of a higher viscosity non-solvent (propylene glycol (PG) or triacetin (T)) can be reduced by adding a lower viscosity additive (such as BA).

In this experiment, we demonstrate that BA can significantly reduce the viscosity of solvent mixtures containing PG and T. This technique can be used to understand the viscosity of other non-solvent blends.

### Injection Force Experiments

Injection force experiments were carried out on 450 mg/mL BSA suspensions in Triacetin: DMAc, and Triacetin: DMAc: BA blends. Injections were carried out at 1 mL/min through a 26 G ½ inch needle.

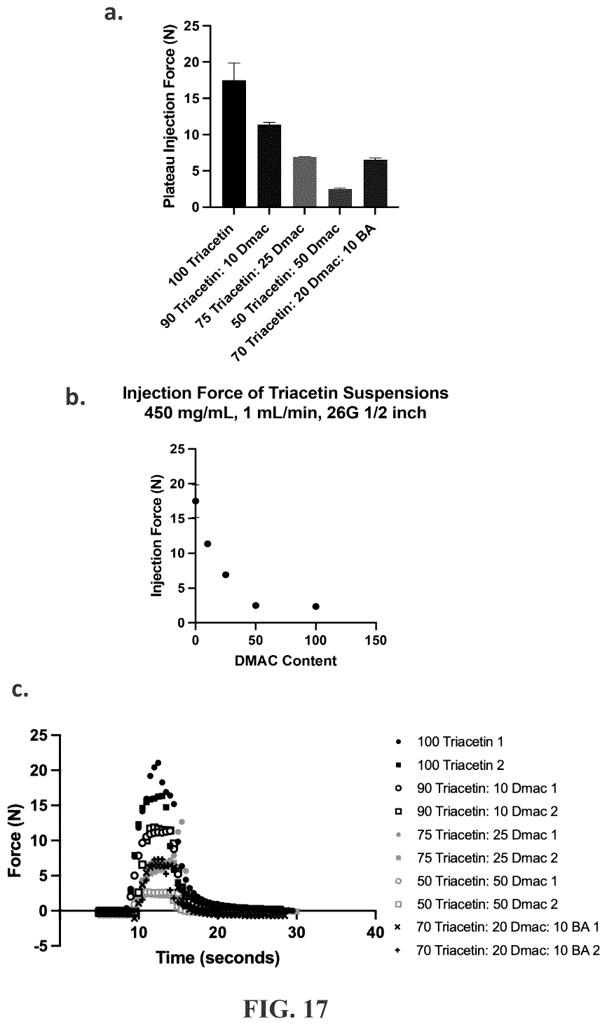

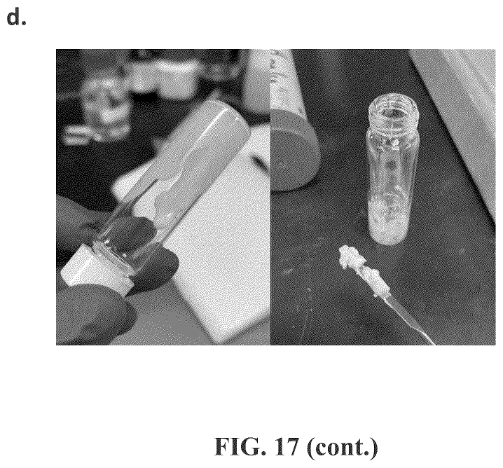

FIG. 17, panels a-d. Injection force measurements (1 mL/min through a 26 G, ½ inch needle) show a significant reduction in injection force when DMAc is used in combination with triacetin as a non-solvent. Panel a shows the plateau injection force for triacetin suspensions (100%) vs suspensions in various blends of triacetin: DMAc and triacetin: DMAc: BA. Panel b shows the injection force of triacetin suspensions vs DMAc content. Panel c shows the injection force of triacetin suspensions (100%) vs suspensions in various blends of triacetin: DMAc and triacetin: DMAc: BA over time. Panel d shows that the addition of polymer MoNi affects the consistency of BSA powder dispersed in DMAc. The left image of panel d illustrates a formulation having 450 mg/mL BSA DMAc including MoNi. The right image of panel d illustrates a formulation having 450 mg/mL BSA in DMAc without the addition of MoNi.

This data shows that adding lower viscosity non-solvents is also advantageous for lowering injection force. Here a 2.5-fold reduction in injection force is observed compared to triacetin alone when DMAc is used at a ratio of 75:25 v/v. A 7-fold reduction in injection force is observed compared to triacetin when DMAc is used alone. This injection force measurement technique can be used to understand the injectability of other non-solvent blends. A blend including triacetin: DMAc: BA (70:20:10) also demonstrates reduced injection force.

### 6.1.9. Example 9—Tolerability of Low-Viscosity Non-Solvent Additives

A tolerability screen was used to quantify the tolerability of injecting low-viscosity, non-solvent additives including benzoyl alcohol (BA), NMP, and DMAc.

SKH1-E mice were subcutaneously administered 10 μL of solvent or solvent blend with triacetin (no protein). Degree of skin reaction to the subcutaneously injected solvent was quantified over the next 24 hours. Volume of solvent injected was 2× the solvent volume that would be administered to a mouse in a paste.

Degree of adverse skin reaction was quantified using the grading scale of Table 5:

TABLE 5
Degree of adverse skin reaction grading scale

| Score | Grade | Description |
|---|---|---|
| 0 | None | No swelling, normal pink |
| 1 | Minimal | Slight swelling with indistinct |
| | | border and/or light pink |
| | | coloration |
| 2 | Mild | Defined swelling with distinct |
| | | boarder or bright pink |
| | | coloration |
| 3 | Moderate | Defined swelling with distinct |
| | | boarder and bright pink |
| | | coloration |
| 4 | Major | Small ulceration (<1 mm |
| | | diameter) along with |
| | | pronounced swelling (>1 mm) |
| | | or redness |
| 5 | Severe | Large ulceration (>1 mm |
| | | diameter) along with |
| | | pronounced swelling (>1 mm) |
| | | or redness |

Tolerability screen results are shown in Table 6:

TABLE 6
Tolerability Screen Results

| | Time | | | | | |
|---|---|---|---|---|---|---|
| Solvent (10 uL) | 0 hr | 30 min | 1 hr | 3 hr | 6 hr | 24 hr |
| 100 Triacetin | 0 | 0 | 0 | 0 | 0 | 0 |
| 100 DMAc | 2 | 4 | 2 | 1 | 1 | 1 |
| 50:50 Triacetin:DMAc | 1 | 1 | 0 | 0 | 0 | 0 |
| 70:30 Triacetin:DMAc | 0 | 1 | 0 | 0 | 0 | 0 |
| 75:25 Triacetin:DMAc | 0 | 1 | 0 | 0 | 0 | 0 |
| 80:20 Triacetin:DMAc | 0 | 1 | 0 | 0 | 0 | 0 |
| 100 BA | 2 | 4 | 2 | 1 | 1 | 1 |
| 50:50 Triacetin:BA | 1 | 1 | 0 | 0 | 0 | 0 |
| 70:30 Triacetin:BA | 0 | 1 | 0 | 0 | 0 | 0 |
| 80:20 Triacetin:BA | 0 | 1 | 0 | 0 | 0 | 0 |
| 100 NMP | 2 | 5 | 3 | 2 | 1 | 1 |
| 50:50 Triacetin:NMP | 1 | 4 | 1 | 1 | 1 | 0 |
| 70:30 Triacetin:NMP | 1 | 2 | 2 | 1 | 1 | 1 |
| 80:20 Triacetin:NMP | 1 | 1 | 0 | 0 | 0 | 0 |

Tolerability screen results demonstrated that triacetin is well tolerated. Mice administered triacetin alone showed no skin swelling or redness. Although DMAc or BA alone resulted in skin irritation, blends with less than or equal to 50:50 v/v DMAc or BA in triacetin were also well tolerated. These blends demonstrated adverse skin reaction grades of less than or equal to 1 in the first 24 hours after subcutaneous injection. NMP subcutaneous injection resulted in greater skin irritation than DMAc or BA.

Tolerability studies demonstrate triacetin, DMAc, and BA are promising non-solvents for in-vivo use. Initial in-vivo work focused on triacetin and DMAc due to this blend's improved slurry stability and lower injection forces.

### 6.1.10. Example 10—In-Vivo Experiments Demonstrating Subcutaneous Administration of BSA Protein Slurries

Fluorescently tagged BSA particles were obtained by spray drying AF647-BSA with untagged BSA at a ratio of 1:500. MoNi was kept at 5 wt % in the final particle formulation. Fluorescently tagged BSA particles were then further diluted with untagged BSA particles with MoNi at a ratio of 1:5 resulting in a final ratio of 1:2500 AF647-BSA: untagged BSA. Protein suspensions in triacetin and 70 triacetin: 30 DMAc were formulated as described previously herein. A bolus control consisted of BSA at a ratio of 1:2500 AF647-BSA: untagged BSA dissolved in PBS at 20 mg/mL.

### A) Protein Slurries in Triacetin Media

SKH1-E mice were subcutaneously administered either 200 μL of 20 mg/mL BSA dissolved in PBS (bolus injection) or 9 μL of 450 mg/mL¹ BSA in a triacetin slurry (high concentration paste or high concentration protein suspension) (total 4 mg BSA in both groups). The BSA slurry was administered using a Hamilton syringe and a 27 G ½ inch needle. The BSA administered to the animals was tagged with an Alexa 647 dye that allowed for fluorescent imaging of the protein remaining in the subcutaneous space at the excitation wavelength of 600 nm and the emission wavelength of 670 nm. The total fluorescent intensities corresponding to each animal were plotted over time between hour 1 and hour˜24 and were fit to a single-phase exponential decay model. Half-lives for subcutaneous absorption were acquired and averaged using GraphPad Prism. ¹Concentration of BSA assumes a particle density of 1 g/cm\hat{ }3. See Example 11 below with respect to BSA particle density. Concentration of BSA estimated to be closer to 506 mg/mL.

### B) Protein Slurries in 70:30 Triacetin: DMAc Media

SKH1-Elite Mice were each given subcutaneous injections of either 8.8 μL of 450 mg/mL fluorescently-tagged BSA protein suspension (in triacetin or 70 triacetin: 30 DMAc) or 200 μL of 20 mg/mL fluorescently tagged BSA in PBS. Protein suspension injections were administered with a 50 μL Hamilton syringe with a 26 G ½ inch needle. Bolus injections were administered with a 1 mL luer lock syringe with a 26 G ½ inch needle. The subcutaneous injection sites of animals were imaged using the IVIS (Lago) over a series of time points spanning two days.

When imaged, mice were anesthetized with isoflurane gas and imaged with an exposure time of 2 s, excitation wavelength of 600 nm, and emission wavelength of 670 nm (binning, medium; F/stop, 1.2). Total radiant efficiency [(photons/s)/(μW/cm2)] was quantified using an equal-sized region of interest surrounding the injection site. As early time points showed fluorescence in the region of interest to increase instead of decrease due to quenching effects, fluorescent intensity at each time point was normalized to fluorescent intensity at the time point of first fluorescent decrease. Normalized fluorescence intensity values for each mouse (n=3-5) were fit to a single exponential decay model, and half-lives were acquired and averaged using GraphPad Prism.

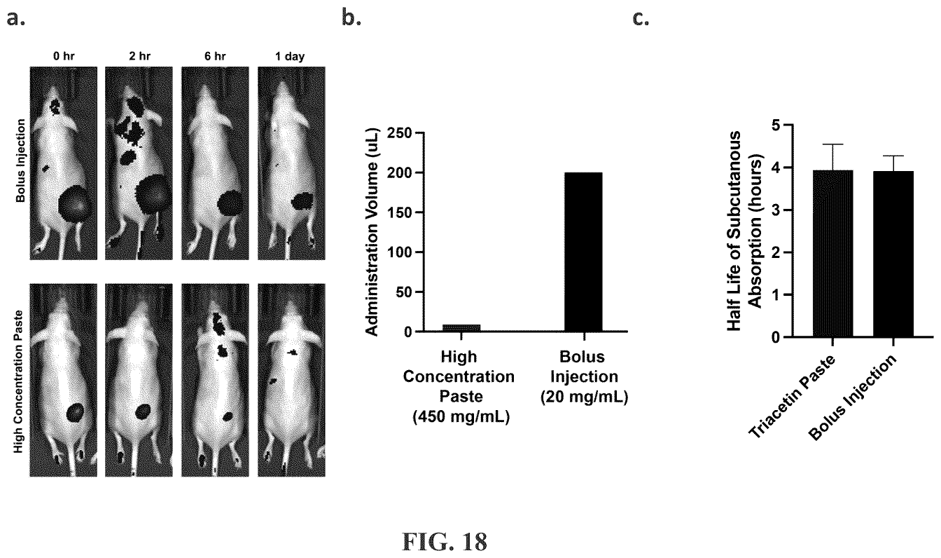

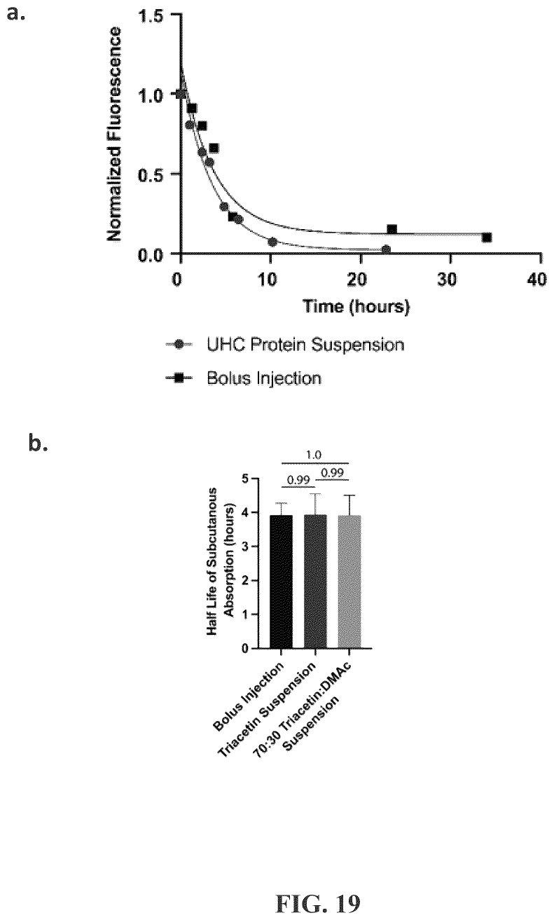

FIG. 18, panels a-c and FIG. 19, panels a-b show that the subject high concentration BSA suspensions allow for in-vivo administration and subcutaneous absorption.

FIG. 18, panels a-c show the results of in-vivo administration to mice of 4 mg BSA by either 200 injections of 20 mg/mL BSA in PBS (bolus injection), or 9 μL injections of 450 mg/mL BSA in triacetin (high concentration paste/high concentration protein suspension) (N≥3 per group). (Panel a) Representative IVIS images demonstrating subcutaneous absorption of fluorescently-tagged BSA administered via a PBS bolus injection or a high concentration protein suspension. (Panel b) Comparative volume of administration for bolus injection and high concentration protein suspension. (Panel c) Half-life of subcutaneous absorption of high concentration paste and bolus injection.

Results show that despite large differences in necessary injection volumes, paste and bolus injections show comparable subcutaneous absorption kinetics with an average half-life of absorption of 4 hours (FIG. 18, panel c, N≥3 per group). It is interesting to note that animals receiving PBS bolus injections appeared to have more residual fluorescence at late time points (FIG. 18, panel a). This could be indicative of poorer total absorption when given high volume injections.

FIG. 19, panels a-b show the results of in vivo administration of 9 μL of 450 mg/mL BSA in a 70:30 triacetin: DMAc slurry. Panel a is fluorescent signal in the subcutaneous space fit to a single phase exponential decay mode to identify half-life of subcutaneous absorption. Panel b shows comparative half-life of subcutaneous absorption for BSA administered via PBS bolus injection or protein suspensions formulated with triacetin or 70 Triacetin: 30 DMAc.

Results showed comparable subcutaneous absorption kinetics between 70:30 triacetin: DMAc slurries and 100% triacetin slurries with an average half-life of absorption of 4 hours. This half-life is similar to the half-life of a PBS bolus injection (20 mg/mL BSA in PBS).

### 6.1.11. Example 11—Measurement of BSA Particle Density

The grain density of spray dried particles can be measured using a Micromeritics pycnometer. Powder mass is measured at the beginning of the run, and the pycnometer measures the volume of the cell. Pycnometer measurements of BSA powder with MoNi result in a powder mass of 158.68 mg, and a powder volume of 0.1208 cm\hat{ }3 (SD: 0.002 cm\hat{ }3). This is an estimated density of 1.31 g/cm\hat{ }3. Density results are consistent across multiple powder batches and multiple runs.

All reported protein concentrations discussed herein assume a spray dried particle density of 1. Since powders incorporated into the suspensions were measured by mass and assumed to take up a volume based on the density of 1 g/cm\hat{ }3, a higher spray dried particle density means the disclosed pastes are likely at higher protein concentrations than originally estimated. Table 7 shows the recalculation of protein concentrations.

TABLE 7
Recalculation of protein concentrations

| Original estimate based on spray | Estimate based on spray dried |
|---|---|
| dried particle density of 1 g/cm\hat{ }3 | particle density of 1.31 g/cm\hat{ }3 |
| 450 mg/mL | 506 mg/mL |
| 460 mg/mL | 519.5 mg/mL |

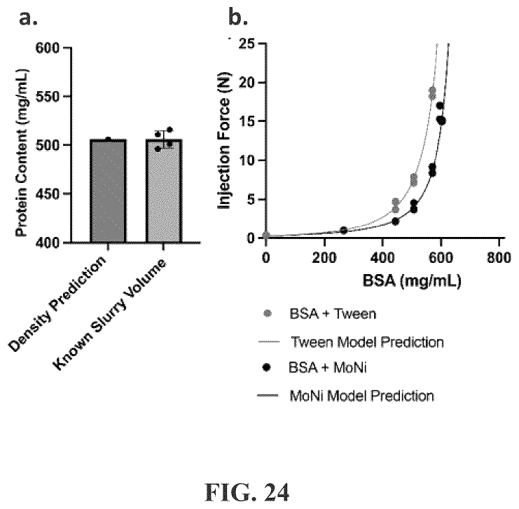

FIG. 24, panels a-b: Panel a depicts comparative protein concentration as predicted from density measurements and measured via nanodrop from a known slurry volume. Panel b shows injection force as a function of concentration for BSA particles (with mol % matched MoNi or Tween 80) in 70 Triacetin: 30 DMAc. Injection force as a function of particle concentration is fit to a particle jamming model. The left column of FIG. 24, panel a is generated by adjusting the math based on the density of 1.31 g/cm\hat{ }3, and the right column is generated by taking a known volume of slurry, dissolving in water, and measuring the actual protein content with UV-vis spectroscopy.

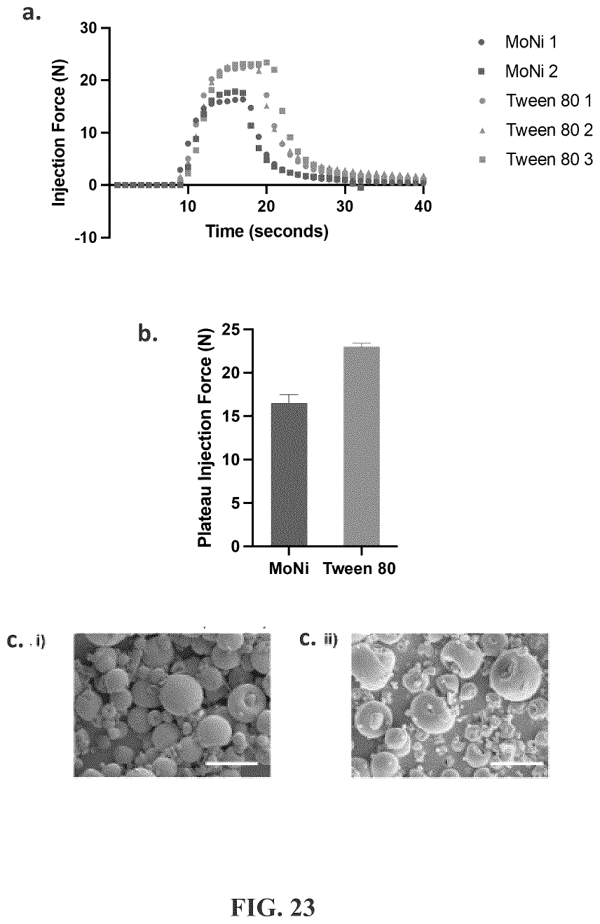

BSA particles spray dried with an equal molar ratio of Tween 80 exhibit similar size and surface morphology (see, FIG. 23, panel c), but as seen in FIG. 24, panel b, higher injection forces at equal BSA concentration. FIG. 24, panel b Injection force measurements with BSA-MoNi in 70 Triacetin: 30 DMAc demonstrate injection of 600 mg/mL BSA with a clinically relevant injection force of 17 N through a 26 G ½ inch needle at 1 mL/min. Concentration vs injection force data was fit to a particle jamming model (Mueller, S. et al., P Roy Soc a-Math Phy 466, 1201-1228 (2010); Krieger, I. M. et al. T Soc Rheol 3, 137-152 (1959)), and using a spherical shape factor, it was found that BSA-MoNi particles achieve close to the theoretical maximum packing density for spheres (volume fraction=0.74) before jamming (FIG. 24, panel b). By particle jamming, and lower maximum injectable concentrations (FIG. 24, panel b, and FIG. 23, panel a-b).

### 6.1.12. Example 12—Comparison to Polysorbate and Pluronic Controls

Polysorbates and pluronics are non-ionic surfactants that can be added to spray drying processes to reduce surface tension and improve protein stability. Polysorbate 80 (Tween 80) and Pluronic L-61 were selected as controls. These pluronic and polysorbate controls were formulated to match the molar ratio of MoNi used in current formulations. Table 8 summarizes the formulations containing each additive.

TABLE 8
MoNi vs Control formulations

| | | Additive | | Total | Protein | |
|---|---|---|---|---|---|---|
| | | Mass in | | Solu- | Content | |
| | Additive | Formu- | Protein | tion | in Spray | |
| | MW | lation | Mass | Volume | Dried | Yield |
| Additive | (g/mol) | (mg) | (g) | (mL) | Product | (%) |
| MoNi | 7000 | 250 | 5 | 250 | 0.952 | 80% |
| Polysor- | 1227 | 43.8 | 5 | 250 | 0.991 | 60% |
| bate 80 | | | | | | |
| Pluronic | 2000 | 71.4 | 5 | 250 | 0.985 | 3% |
| L-61 | | | | | | |

Without being bound to any particular theory, it is hypothesized that MoNi may be a preferable spray drying additive to polysorbate 80 or Pluronic L-61 because of its higher glass transition temperature. MoNi has a glass transition temperature of 135° C. while Tween 80 and Pluronic L-61 have glass transition temperatures well below room temperature. A higher glass transition temperature may result in improved stability through the spray drying process and a less tacky product as well as higher yields.

### Powder Quality and Yield

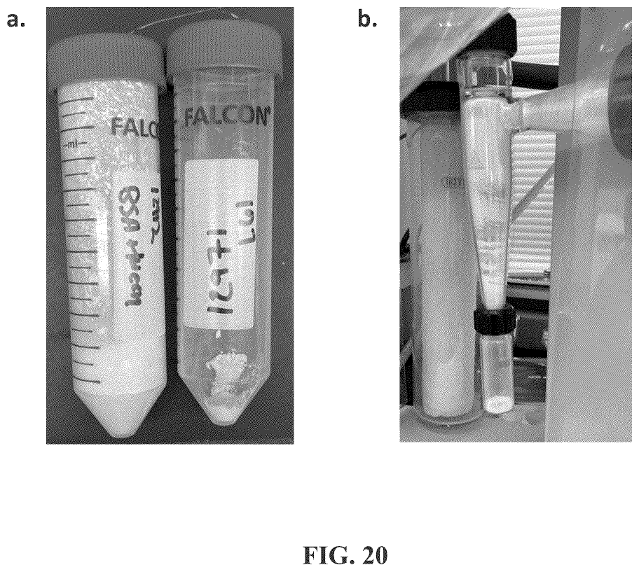

FIG. 20, panels a-b illustrate the products obtained after spray drying formulations containing polysorbate 80 and Pluronic L-61. Panel a shows images of falcon tubes containing comparative products recovered from spray drying equal mass of BSA with polysorbate 80 (left tube) and Pluronic L-61 (right tube). Panel b shows the spray dryer glass component after spray drying Pluronic L-61. As evident, in the image, spray drying Pluronic L-61 results in a tacky powder that sticks to the spray dryer glass components, thus resulting in a poor yield of the spray dried product (see panel a, right tube).

### Powder Stability Through Spray Drying Process

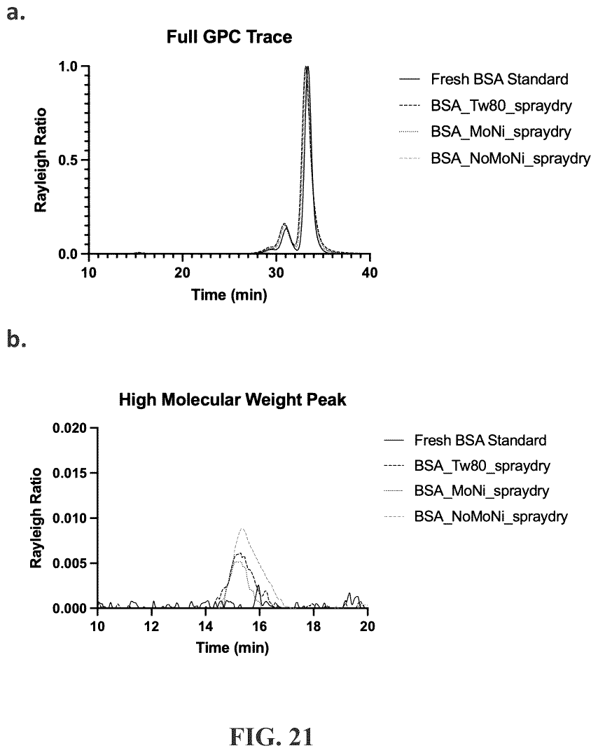

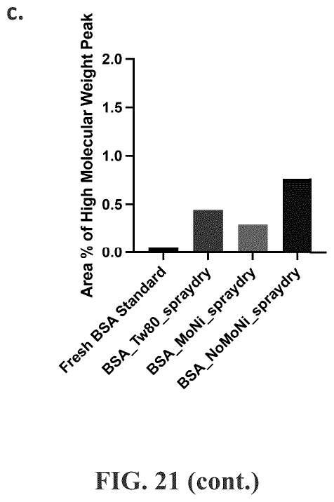

FIG. 21, panels a-c illustrate SEC traces for fresh BSA and BSA (Fresh BSA Standard) spray dried with MoNi (BSA_MoNi), polysorbate 80 (BSA_Tw80), and no additive (BSA_NoMoNi) (Samples were run on a Superose 6 10/300 GL column at 0.5 mL/min). Panel a shows for all spray dried samples, the monomer and dimer peaks appear similar. Panel b shows differences between the spray dried samples for the high molecular weight aggregate peaks. Panel c shows the Area fraction of the high molecular weight peak is smallest for fresh BSA standard, followed by BSA spray dried in MoNi, BSA spray dried with polysorbate 80, and finally BSA spray dried without an additive.

SEC traces suggest that MoNi is beneficial in preserving protein stability during the spray drying process. Samples spray dried with MoNi showed smaller high molecular weight peaks (likely corresponding to aggregates) than samples spray dried with either polysorbate 80 or no additive. BSA is a very stable protein, but one would expect to see more pronounced effects if the stability of a more challenging protein to spray dry was examined, such as a monoclonal antibody.

Stressed Aging Comparison of Powder Formulated with MoNi and Polysorbate 80

To understand the stability of protein pastes made with BSA, BSA with MoNi, and BSA with polysorbate 80, pastes at 450 mg/mL BSA in triacetin were aged at 60° C. for 60 minutes. BSA at 20 mg/mL in PBS aged under the same conditions was used as a positive control.

Following stressed aging, samples were run on the SEC to look for changes in the area fraction of the high molecular weight aggregate peak.

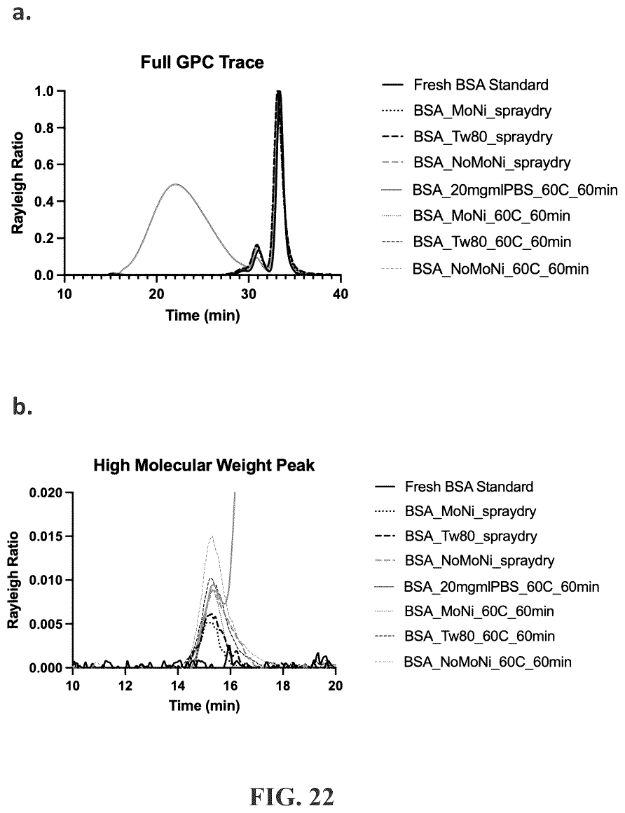

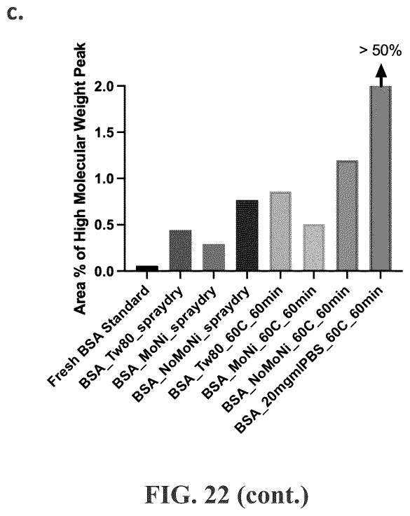

FIG. 22, panels a-b illustrate full SEC traces and traces of high molecular weight aggregate peaks for aged protein pastes made with BSA, BSA with MoNi, and BSA with polysorbate 80. BSA aged at 20 mg/mL in PBS was used as a control. All samples were aged at 60° C. for 60 minutes. Samples were run on a Superose 6 10/300 GL column at 0.5 mL/min. Table 9 shows the sample key for FIG. 22. Panel a shows differences between the samples for the high molecular weight aggregate peaks. Panel b shows the Area percentage of the high molecular weight peaks for each sample.

TABLE 9
Sample Key for FIG. 21

| | | | Solvent/ | Concen- |
|---|---|---|---|---|
| | Spray | | Non | tration |
| Sample | dried? | Aged? | Solvent | (mg/mL) |
| Fresh BSA Standard | no | no | N/A | N/A |
| BSA_MoNi_spraydry | yes | no | N/A | N/A |
| BSA_Tw80_spraydry | yes | no | N/A | N/A |
| BSA_NoMoNi_spraydry | yes | no | N/A | N/A |
| BSA_20 mgmlPBS_60 C._{—} | no | yes | PBS | 20 |
| 60 min | | | | |
| BSA_MoNi_60 C._60 min | yes | yes | Triacetin | 450 |
| BSA_Tw80_60 C._60 min | yes | yes | Triacetin | 450 |
| BSA_NoMoNi_60 C._60 | yes | yes | Triacetin | 450 |
| min | | | | |

SEC traces suggest that MoNi is beneficial in preserving protein stability during the aging process. Aged Samples containing MoNi showed smaller high molecular weight peaks than samples containing polysorbate 80 or no additive. The 20 mg/mL control in PBS showed significant aggregation during the stressed aging experiment.

Injectability Comparison of Powder Formulated with MoNi and Polysorbate 80

FIG. 23, panels a-c show injection force experiments (1 mL/min through a 26 G ½ inch needle) with 450 mg/mL BSA suspensions in triacetin. The BSA has been spray dried with either polysorbate 80 (Tween 80) or MoNi at equal molar amounts (7.14 μmol per gram of BSA). Panel a shows the injection force (N) over time (seconds). Panel b shows the plateau injection force for the formulation including MoNi vs polysorbate 80 (Tween 80). Panel c illustrates SEM of particle morphology for i) BSA MoNi and ii) BSA polysorbate 80 (Tween 80) particles (scale bars are 10 μm).

The results show that when formulated with equal molar ratios of MoNi and polysorbate 80 (7.14 μmol per gram of BSA), pastes formulated with polysorbate 80 show a 43% increase in injection force. It is relevant to note that MoNi is much less toxic than polysorbate 80, allowing for a larger formulation design space.

### 6.1.13. Example 13—Bovine Serum Albumin (BSA) Microparticle Suspensions

Aqueous formulations of bovine serum albumin (BSA) were prepared and spray dried under the conditions in Table 10 to provide BSA microparticle formulations.

TABLE 10
Process parameters for preparation
of BSA microparticle formulations

| Formulation # | 004A | 004B |
|---|---|---|
| Solution Formulation | BSA 10 mg/mL, | BSA 10 mg/mL, |
| | MoNi 0.5 mg/L | MoNi 1 mg/L |
| Solvent system | Milli-Q Water | Milli-Q Water |
| Solids Loading (wt %) | 1.039% | 1.088% |
| Batch Size (BSA in g) | 20 | 20 |
| Drying Gas Feed Rate | 0.5 | 0.5 |
| (m³/min) | | |
| Liquid Feed Rate (mL/min) | 5.0 | 5.0 |
| Approximate Processing Time | 6.67 | 6.67 |
| (hours) | | |
| Atomization Pressure (bar) | 4.0 | 4.0 |
| Drying Gas Inlet Temperature | 71.5 | 71.5 |
| (° C.) | | |
| Spray Dryer Outlet Temperature | 40.0 | 40.0 |
| (° C.) | | |

Microparticle formulations 004A and 004B were imaged by scanning electron microcopy (SEM), and both formulations were observed to exhibit smooth particles of less than or equal to 5 μm.

FIG. 25, panels a-b show the SEM images of the BSA microparticle formulations. Panel a shows the SEM image of the 004A formulation (smooth particles≤5 μm). Panel b shows the SEM image of the 004B formulation (smooth particles≤5 μm).

Formulations 004A and 004B were suspended in triacetin and loaded into 1 mL Schott syringes (27 G thin-walled needle and V9519 coated plunger). Both formulations exhibited a consistent suspension and no particle settling or phase segregation was observed.

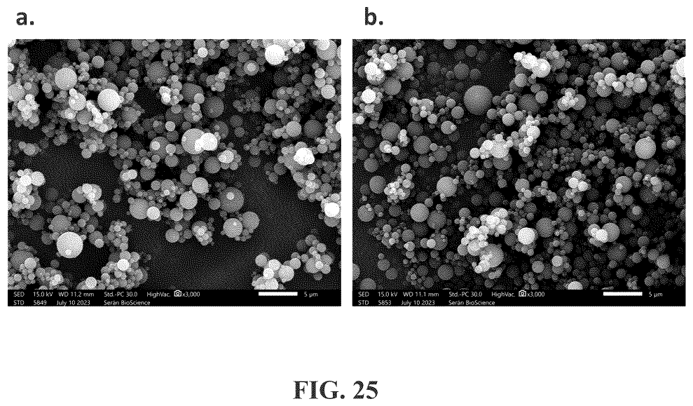

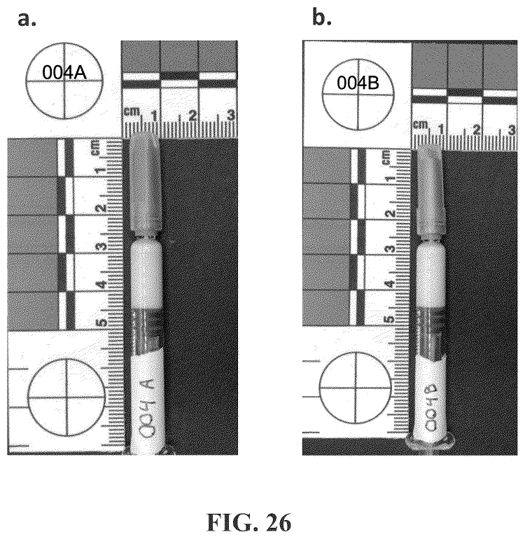

FIG. 26, panels a-b show photographic images of the BSA microparticle formulations suspended in triacetin and loaded in 1 mL Schott syringes. Panel a shows a photographic image of the syringe loaded with 004A (480 mg/mL) formulation suspended in triacetin. Panel b shows a photographic image of the syringe loaded with 004B (458 mg/mL) formulation suspended in triacetin.

To investigate the injectability of the BSA microparticle formulations, injection force measurements were taken for formulations 004A and 004B suspensions in triacetin injected at different flow rates from 1 mL Schott syringes (27 G thin-walled needle and V9519 coated plunger).

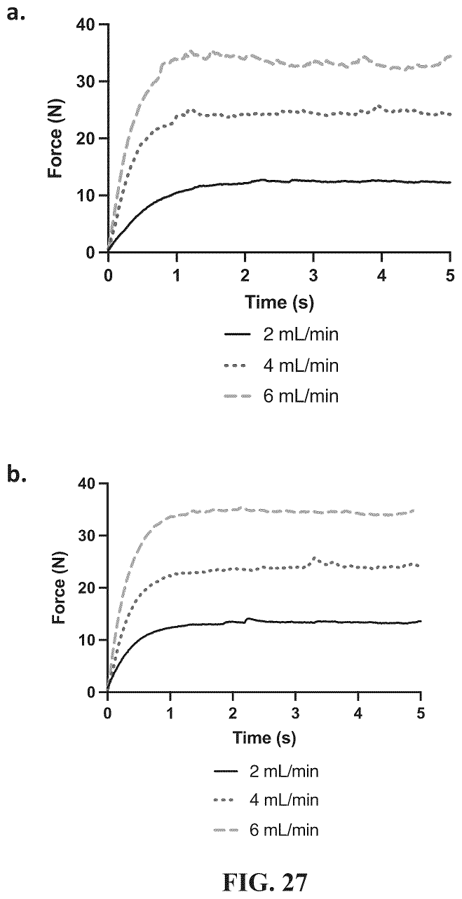

FIG. 27, panels a-b are graphs of the injection force (N) of the BSA microparticle suspensions over 5 seconds at various flow rates. Panel a shows injection force (N) over 5 seconds for BSA microparticle suspension 004A (480 mg/mL BSA) in triacetin injected at 2 mL/min, 4 mL/min and 6 mL/min from a 1 mL Schott syringe (27 G thin-walled needle and V9519 coated plunger). Panel b shows injection force (N) over 5 seconds for BSA microparticle suspension 004B (458 mg/mL BSA) in triacetin injected at 2 mL/min, 4 mL/min and 6 mL/min from a 1 mL Schott syringe (27 G thin-walled needle and V9519 coated plunger).

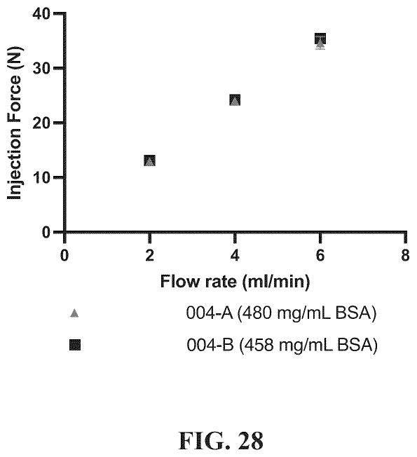

FIG. 28 illustrates the injection force measurements of 004A (480 mg/mL BSA) and 004B (458 mg/mL) microparticle suspensions in triacetin injected at different flow rates from 1 mL Schott syringes (27 G thin-walled needle and V9519 coated plunger). This graph shows that injection force (N) for suspensions of 004A and 004B in triacetin is linear with flow rate.

### 6.1.14. Example 14—In-Vivo Experiments Demonstrating Subcutaneous Administration of Human Immunoglobulin G (hIgG) Protein Slurries

Following the success with BSA, the in-vivo experiments were applied to human immunoglobulin G (hIgG). It is noted that hIgG is directly comparable with mAb therapeutics in both chemical structure, solubility and size.

Human immunoglobulin G (hIgG) was fluorescently labeled with Alexa-647-NHS from lumiprobe at 5% wt ratio. In brief, 50 mg dry hIgG was dissolved in 10 mL of PBS and 500 μL of a 5 mg/mL DMSO stock solution of Alexa-647-NHS was added to the solution. The reaction proceeded for 24 h at room temperature and the free dye was removed with 10 kDa MWCO amicon spin filter.

Fluorescently tagged hIgG particles were obtained by spray drying Alexa Fluor 647 dye (AF647)-tagged hIgG with untagged BSA at a ratio of 1:20. MoNi was kept at 5 wt % in the final particle formulation. Fluorescently tagged hIgG particles were then further diluted with untagged hIgG particles with MoNi at a ratio of 1:100 resulting in a final ratio of 1:2000 AF647-tagged hIgG: untagged hIgG. Protein suspensions in triacetin were formulated as described above. A bolus control consisted of hIgG at a ratio of 1:2000 AF647-tagged hIgG: untagged hIgG dissolved in PBS at 100 mg/mL.

SKH1-Elite Mice were each given subcutaneous injections of either 20 μl of 300 mg/mL fluorescently-tagged hIgG protein suspension in triacetin or 60 μL of 100 mg/mL fluorescently tagged hIgG in PBS. Protein suspension and bolus injections were both administered with an insulin syringe with a 28 gauge needle. The subcutaneous injection sites of animals were imaged using the IVIS (Lago) over a series of time points spanning two days.

When imaged, mice were anesthetized with isoflurane gas and imaged with an exposure time of 1 s, excitation wavelength of 600 nm, and emission wavelength of 670 nm (binning, medium; F/stop, 1.2). Total radiant efficiency [(photons/s)/(μW/cm2)] was quantified using an equal-sized region of interest surrounding the injection site. As early time points showed fluorescence in the region of interest increasing instead of decreasing due to quenching effects, fluorescent intensity at each time point was normalized to fluorescent intensity at the time point of first fluorescent decrease. Normalized fluorescence intensity values for each mouse (n=3-5) were fit to a single exponential decay model, and half-lives were acquired and averaged using GraphPad Prism.

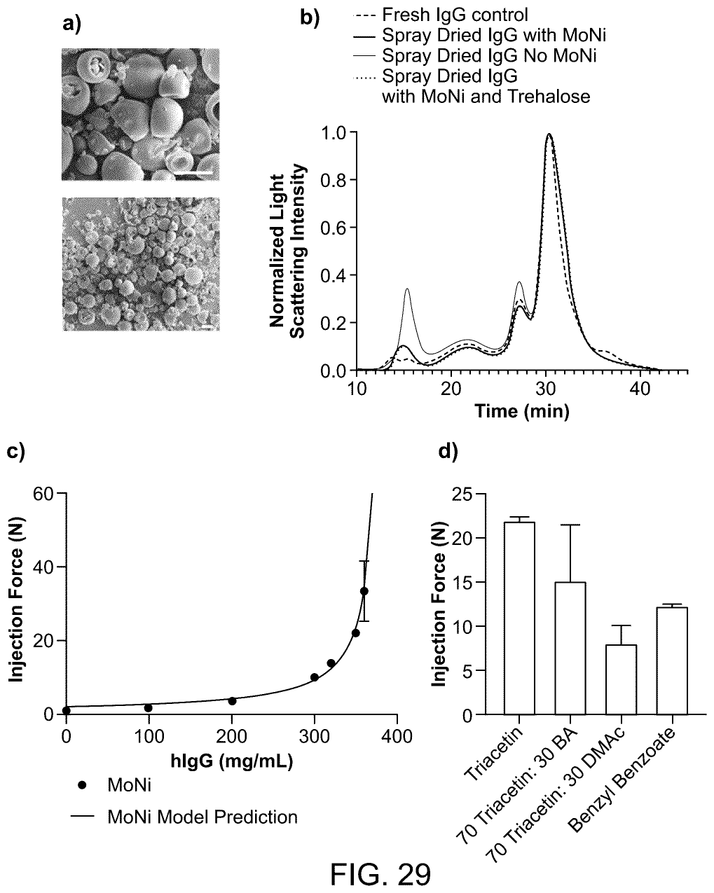

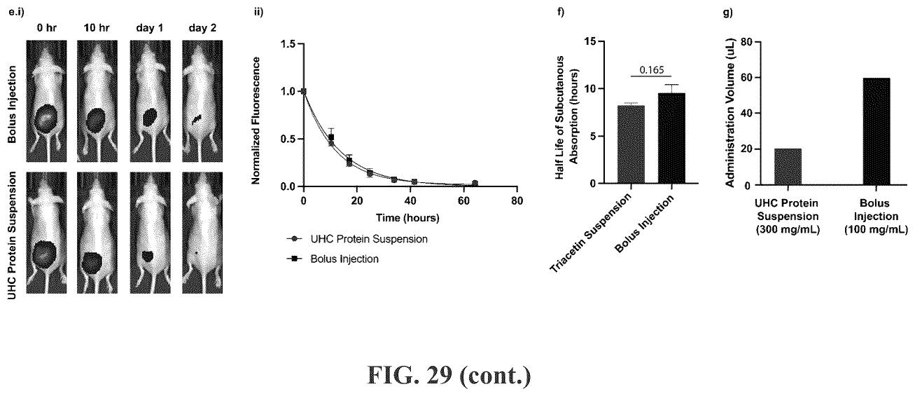

FIG. 29, panels a-g. Illustrate that high concentration suspension technology can be effectively used to deliver hIgG. Panel a depicts a SEM of spray dried IgG with MoNi particle morphology (scale bars are 10 μm). Panel b depicts a SEC trace of fresh hIgG control, spray dried hIgG without MoNi, spray dried hIgG with 5 wt % MoNi, spray dried hIgG with 25 wt % trehalose and 5 wt % MoNi. PBS with sodium azide is used as an eluent. Panel c shows injection force as a function of concentration for hIgG microparticles with MoNi in triacetin. Injection force as a function of particle concentration is fit to a particle jamming model. (Panel d) Injection force for injection of 350 mg/mL hIgG, 5 wt % MoNi suspensions at 1 mL/min through 26 G ½ inch needles with DMAc, benzyl alcohol (BA) and benzyl benzoate (BB) non-solvent additives. (Panel e. i) Representative IVIS images demonstrating subcutaneous absorption of fluorescently-tagged hIgG administered via a PBS bolus injection or a high concentration protein suspension. (Panel e. ii) Fluorescent signal in the subcutaneous space is fit to a single phase exponential decay mode to identify half-life of subcutaneous absorption. (Panel f) Comparative half-life of subcutaneous absorption for hIgG administered via PBS bolus injection or UHC protein suspensions formulated with triacetin. (Panel g) Comparative volume of administration for bolus injection and high concentration protein suspension.

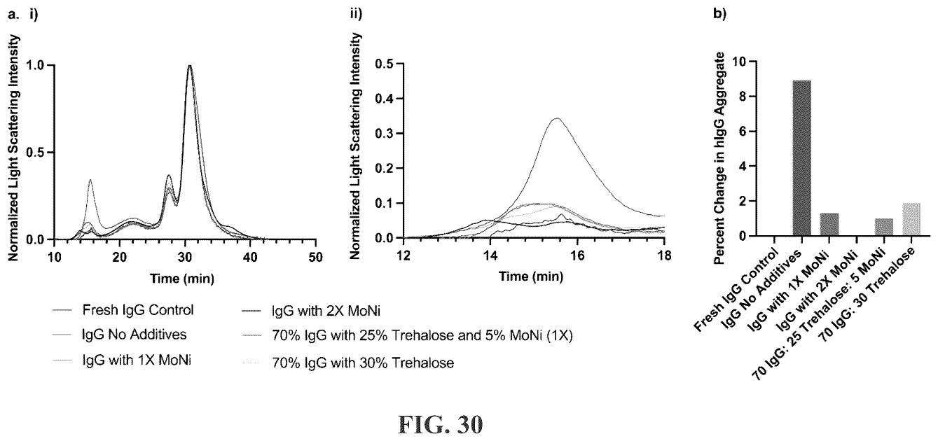

Similar to the BSA formulations described in Example 10, the hIgG to MoNi ratios in the final spray dried product were fixed at 20:1, initial feed stock concentrations and spray drying parameters were optimized to produce spherical particles. As shown in FIG. 29, panel a, spray dried hIgG particles with 5 wt % MoNi are 5-20 μm in diameter and have a spherical morphology. As previously observed with BSA, MoNi stabilized hIgG during the high-temperature spray drying process. SEC characterization of fresh hIgG as well as hIgG spray dried with and without MoNi shows that spray drying hIgG with MoNi results in a higher monomer peak fraction and reduced high molecular weight aggregates (FIG. 29, panel b). Importantly, a control hIgG formulation utilizing 30 wt % trehalose as stabilizer was found to have similar aggregate fraction as hIgG with 5 wt % MoNi, and the addition of trehalose at 25 wt % to the 5 wt % MoNi formulation afforded no additional stability benefit to the spray dried product (FIG. 30). In summary, these results show that only 5 wt % MoNi was necessary to stabilize hIgG during the spray drying process compared to the state-of-the-art formulations, which include at a minimum 30% wt trehalose (Maury, M. et al. Eur J Pharm Biopharm 59, 251-261 (2005)).

With these high hIgG content microparticles (95 wt %), we were able to use triacetin as a non-solvent to formulate suspensions and measure injection force as a function of hIgG concentration assuming a particle density of 1 g/cm³. Injection force measurements with hIgG, MoNi in triacetin demonstrate injection of 350 mg/mL hIgG with an injection force of 22 N through a 26 G ½ inch needle at 1 mL/min (FIG. 29, panel c). To understand particle interactions, concentration vs injection force data was again fit to a particle jamming model (Mueller, S. et al., P Roy Soc a-Math Phy 466, 1201-1228 (2010)) using a sphere shape factor (FIG. 29, panel c). Given that the hIgG-MoNi particles have a similar morphology as the BSA-MoNi particles, as determined with SEM imaging, this jamming at lower concentrations implies greater particle-particle interactions, which is likely due to the physical differences of dried hydrophobic antibodies vs. dried hydrophilic albumin. As seen with BSA, injection force of hIgG suspensions can be further reduced by altering non-solvent composition. 70 Triacetin: 30 DMAc and 70 Triacetin: 30 BA reduce injection force for a 350 mg/mL hIgG suspension (22 N) by 2.75-fold (8 N) and 1.5-fold (15 N) respectively. Additionally, benzyl benzoate, a low-viscosity FDA approved excipient often used as a preservative, reduces injection force by 1.8-fold (12 N) (FIG. 29, panel d).

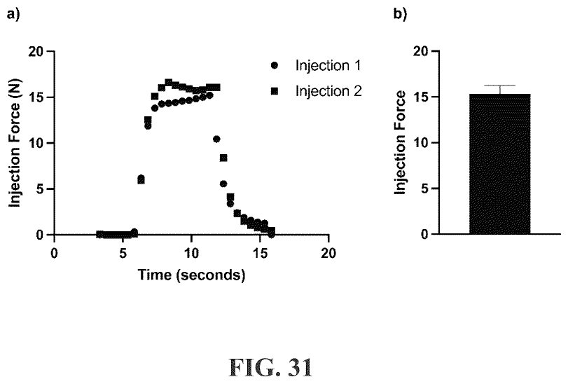

As described above, to demonstrate in-vivo delivery and subcutaneous absorption of hIgG-MoNi suspensions, SKH1e mice were subcutaneously injected through 28-gauge insulin needles with equal doses of fluorescently tagged hIgG in either a 300 mg/mL triacetin suspension, or a 100 mg/mL PBS bolus (FIG. 31 shows injection force curves for injection of 300 mg/mL IgG, 5 wt % MoNi suspensions at 1 mL/min through BD Insulin syringes). The higher-concentration IgG bolus was chosen to be more representative of high-concentration aqueous mAb products. Fluorescent images collected from an in vivo Imaging System (IVIS) were then utilized to study hIgG subcutaneous absorption at the site of injection (FIG. 29, panel e. i)). Fluorescent signal over time from the injected protein suspension and bolus was normalized and fit to a single-phase exponential decay curve to provide a half-life of hIgG subcutaneous absorption (FIG. 29, panel e. ii)). The half-life of hIgG subcutaneous absorption was about 9 hours (FIG. 29, panel f) and agreed with previous literature values (Deng, R. et al. Subcutaneous bioavailability of therapeutic antibodies as a function of FcRn binding affinity in mice. MAbs 4, 101-109 (2012). Half-life of absorption was statistically insignificant between administration methods despite a 3-fold difference in required injection volume (FIG. 29, panel g).

### 6.1.15. Example 15—Insulin and Pramlintide Co-Formulations

A shelf-stable co-formulation of insulin and pramlintide that is injectable through clinically relevant needle gauges was investigated.

Spray drying insulin and pramlintide particles: Feed solutions for spray dried insulin particles were formed by diluting U500 insulin stock with cell grade water containing trehalose and MoNi. The insulin feed solution comprises as follows: insulin (0.99 mg/mL), glycerin (0.91 mg/mL), metacresol (0.14 mg/mL), zinc (0.005 mg/mL) MoNi (2.5 mg/mL) and trehalose (45.4 mg/mL) (sum of 50 mg/mL). Feed solutions for spray dried pramlintide particles were formed by diluting 5 mg/mL pramlintide stock with cell grade water containing trehalose and MoNi. The pramlintide stock solution comprises as follows: pramlintide (0.23 mg/mL), MoNi (2.5 mg/mL) and trehalose (47.3 mg/mL) (sum of 50 mg/mL).

Solutions were spray dried at a concentration of 50 mg/mL solids. Solutions were formulated in the lab and transferred on ice to the spray dryer. Solutions were allowed to warm to room temperature before spray drying. Total time between preparing feed solutions and spray drying was about 4 hours.

Particles were spray dried using a Buchi B-290 spray dryer equipped with a high-performance cyclone. Spray dryer parameters were the following: inlet temp: 130° C., outlet temp: 78° C., 5% pump (2-3 mL/min), 40 mm air, nozzle cleaning 3, room temp: 20° C., room humidity: 42%.

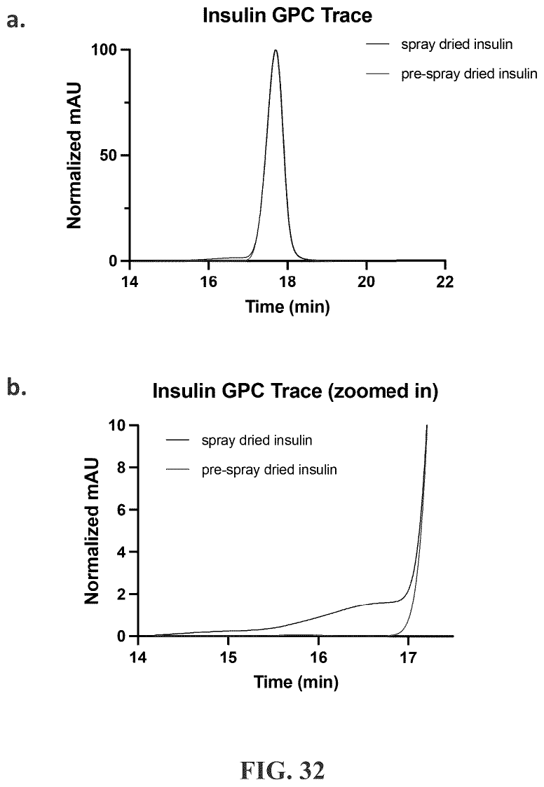

FIG. 32, panels a-b show SEC traces of insulin before and after spray drying. (Panel a) shows the insulin SEC Trace of spray dried insulin and pre-spray dried insulin over 22 minutes. (Panel b) is a zoomed in portion of minutes 14-17 of the SEC trace in panel a.

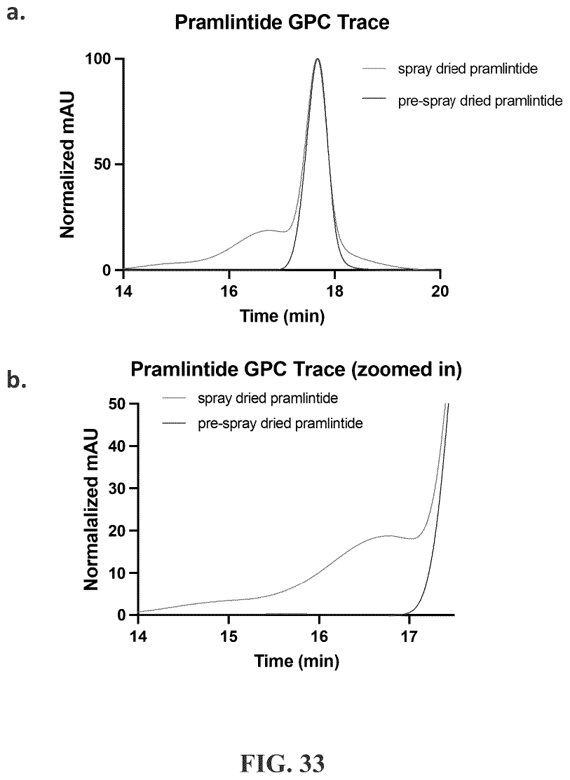

FIG. 33, panels a-b show SEC traces of pramlintide before and after spray drying. (Panel a) shows the pramlintide SEC Trace of spray dried pramlintide and pre-spray dried pramlintide over 20 minutes. (Panel b) is a zoomed in portion of minutes 14-17 of the SEC trace in panel a.

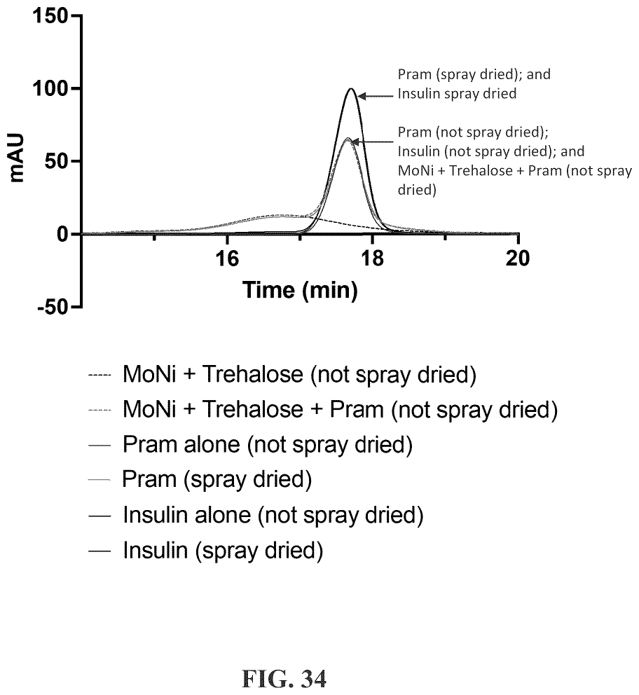

FIG. 34 shows SEC trace of pramlintide (“pram”), insulin, and controls before and after spray drying.

While SEC curves of spray dried insulin and pramlintide initially appear to have high molecular weight tails, additional characterization demonstrates that high molecular weight peaks can be attributed to the high concentration of MoNi in the spray dried powder samples rather than protein aggregation. Samples containing MoNi, trehalose, and pramlintide before and after spray drying show identical SEC traces (FIG. 34). The high molecular weight MoNi peak in the spray dried insulin sample appears smaller than the peak in the pramlintide sample because the SEC sample (formulated at 0.66 mg/mL therapeutic protein) contains only 1.75 mg/mL MoNi compared to the pramlintide SEC sample which contains 7.53 mg/mL MoNi.

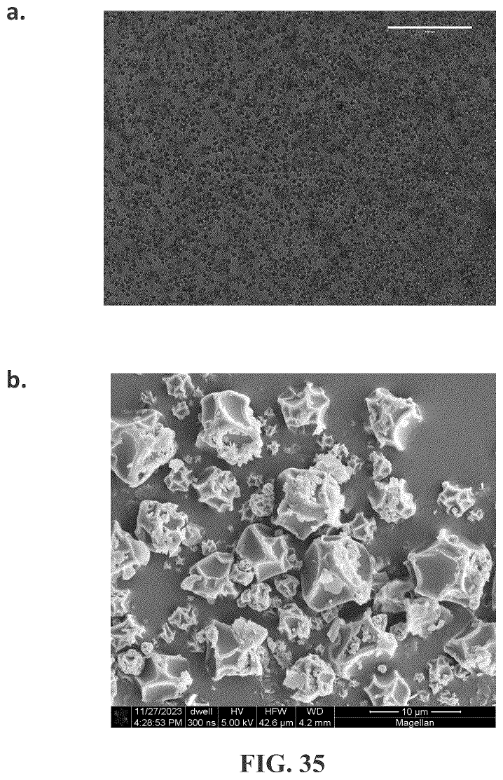

Resuspending insulin and pramlintide particles in triacetin: Insulin and pramlintide spray dried particles were resuspended in triacetin. Particles were left in triacetin for 24 hours before imaging. As expected from prior SEM imaging, particles appear to be 5-20 μm in diameter with most particles less than 10 μm (FIG. 35).

FIG. 35 panels a-b. SEM images of insulin and pramlintide spray dried particles resuspended in triacetin (panel a) and trehalose and MoNi (95:5) particles (panel b). Particles in panel b do not contain either insulin or pramlintide, so protein may affect particle morphology.

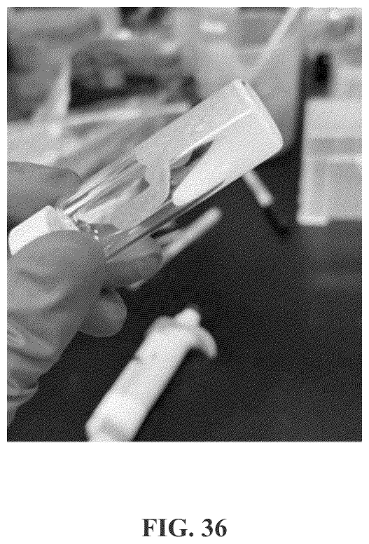

FIG. 36 shows Insulin and pramlintide spray dried particles resuspended in triacetin at a solids content of 350 mg/mL. Particle suspension flows like a liquid.

Co-formulation injectability: The co-formulation of insulin and pramlintide has 350 mg/mL solids. It contains 175 mg/mL insulin powder particles and 175 mg/mL pramlintide powder particles resuspended in triacetin. The final composition results in a standard insulin dose of 3.47 mg/mL (100 insulin units per mL) and a pramlintide dose of 0.8 mg/mL.

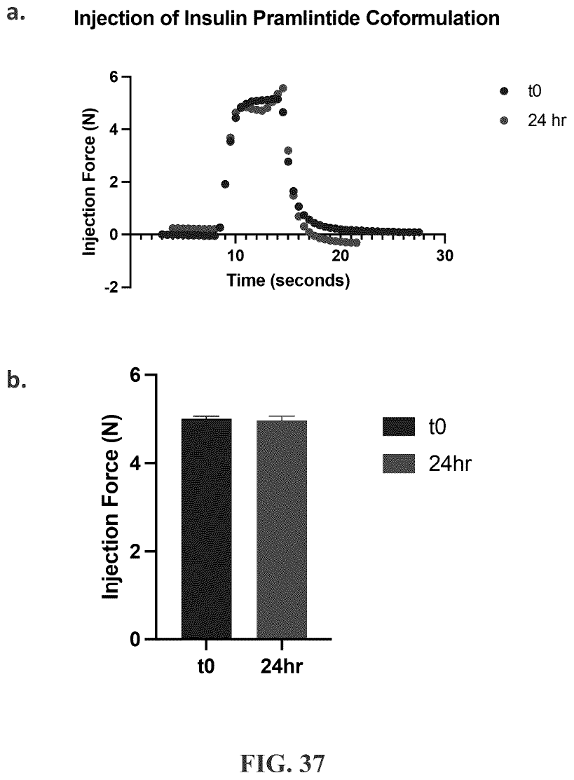

FIG. 37, panels a and b show injection force of insulin pramlintide co-formulation (350 mg/mL solids) in triacetin. Injection measurements were performed using a 1 ml syringe at 1 mL/min through a 26 G ½ inch needle.

The current co-formulation is easily injectable through clinically relevant needle gauges, and no clogging was observed. It may be relevant to increase the overall solids content of the suspension to prevent particle settling in a final formulation, especially if formulations are stored in syringes prior to injection.

Redissolving co-formulation in water: Water was added to the co-formulation paste, and the formulation dissolved clear with little visible aggregation.

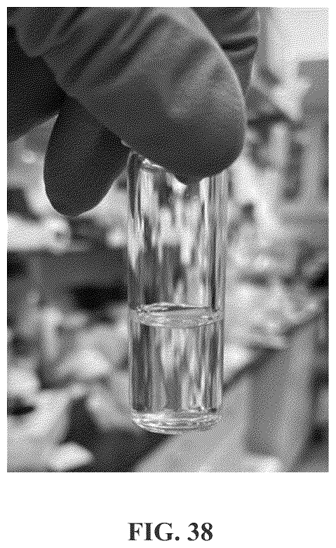

FIG. 38 is an image depicting the insulin pramlintide co-formulation redissolved in cell grade water.

### 6.1.16. Example 16—Syringe Force Measurements of Various Liquid Carriers

To investigate the syringe force profile for various liquid carriers in syringes (e.g., break loose (initiating) force, and the glide (sustaining) force), the following media alone were loaded into syringes in duplicate (n=2):
1. triacetin (100%)
2. benzyl benzoate (100%)
3. Miglyol 810 (100%)
4. Miglyol 840 (100%)
5. benzyl alcohol (100%)
6. ethyl oleate (100%).

The above syringes were stored for 48 h at ambient temperature, protected from light. No visual change was observed comparing the appearances of each at time at loading to the appearance of the same 48 h after storage.

Syringe force measurements (n=2) were conducted on each media after 48 h. The results are shown in Table 11.

TABLE 11
Syringe Force Testing Results

| | Break loose | Glide force | Viscosity |
|---|---|---|---|
| Vehicle | (N), n = 2 | (N), n = 2 | (cp)* |
| triacetin | 8.1 | 6.1 | 23 |
| benzyl benzoate | 5.7 | 3.5 | 8-9 |
| Miglyol 810 | 8.2 | 7.9 | 27-30 |
| Miglyol 840 | 5.7 | 3.7 | 9-12 |
| benzyl alcohol | 4.8 | 2.5 | 5 |
| ethyl oleate | 4.2 | 3.2 | 6 |

Overall, comparing the syringe force profile (lowest to highest):

$$benzyl alcohol ∼ ethyl oleate < benzyl benzoate ∼ Miglyol 840 < triacetin ∼ Miglyol 810 .$$

### 6.1.17. Example 17—BSA and MoNi Suspensions in Prefilled Syringes

The syringe force profiles of injectable formulation including BSA and MoNi suspensions (460 mg/mL) in various liquid carriers. Exemplary formulations were prepared according to General method B (as described herein). Table 12 below summarizes the formulations prepared for this example:

TABLE 12
BSA + MoNi Formulations - Prepared by General Method B

| | | | | | Vol. of | Final |
|---|---|---|---|---|---|---|
| | | | BSA + | | liquid | Conc. |
| | BSA | Polymer | MoNi | Liquid | carrier | of BSA |
| # | (mg) | Additive | (mg) | Carriers | (μL) | (mg/mL)* |
| 12 | 288.4 | MoNi | 303.0 | benzyl | 324 | 460 |
| | | | | benzoate | | |
| 13 | 596.7 | MoNi | 311.6 | Miglyol 840 | 333 | 460 |
| 14 | 290.9 | MoNi | 305.6 | benzyl | 327 | 460 |
| | | | | alcohol | | |
| 16 | 296.4 | MoNi | 311.4 | ethyl | 333 | 460 |
| | | | | oleate | | |

*calculated using 1 g/mL as the density of spray dried powder.

Formulations 12, 13, 14, and 16 were prepared as above, loaded into syringes in duplicate (n=2), and held at ambient temperature for 48 hours protected from light. The appearance of each of the formulations at 48 hours was compared to the appearance at the time of loading. After 48 hours storage in prefilled syringes, the following observations were made:
Formulation 12 (benzyl benzoate): no visual change;
Formulation 13 (Miglyol 840): no visual change;
Formulation 14 (benzyl alcohol): suspension became a solid material; and
Formulation 16 (ethyl oleate): no visual change.

In summary, after 48 hours storage in a prefilled syringe no change was observed for formulations 12, 13 and 16, which include benzyl benzoate, Miglyol 840, and ethyl oleate as liquid carriers, respectively. As such, formulations 12, 13 and 16, after loading into prefilled syringes and stored for 48 hours, were further evaluated to establish syringe force profiles. Prefilled syringes were subjected to force measurements in duplicate (n=2) at a rate of 6 mL/min, and results were calculated using 1.5-2.2 s duration.

The results of the syringe force experiments (n=2, 6 mL/min, average of the two samples tested) are shown in Table 13, along with density, viscosity and UV recovery.

TABLE 13
summary of Syringe Force profiles for Formulations 12, 13 and 16

| | | Avg Glide | | | |
|---|---|---|---|---|---|
| Sample | | Force (N), | Density | Viscosity | Recovery |
| # | Liquid Carrier | n = 2 | (g/mL) | (cp) | % (UV) |
| 12 | Benzyl Benzoate | 24.4 | 1.25 | 115.2 | 91.0% |
| 13 | Miglyol 840 | 27.8 | 1.12 | 125.4 | 91.9% |
| 16 | Ethyl Oleate | 16.8 | 1.09 | 66.78 | 96.7% |

As seen in Table 13, formulation 16 with ethyl oleate as liquid carrier has the lowest syringe force of the formulations tested. Formulations 12 and 13 exhibited similar glide force.

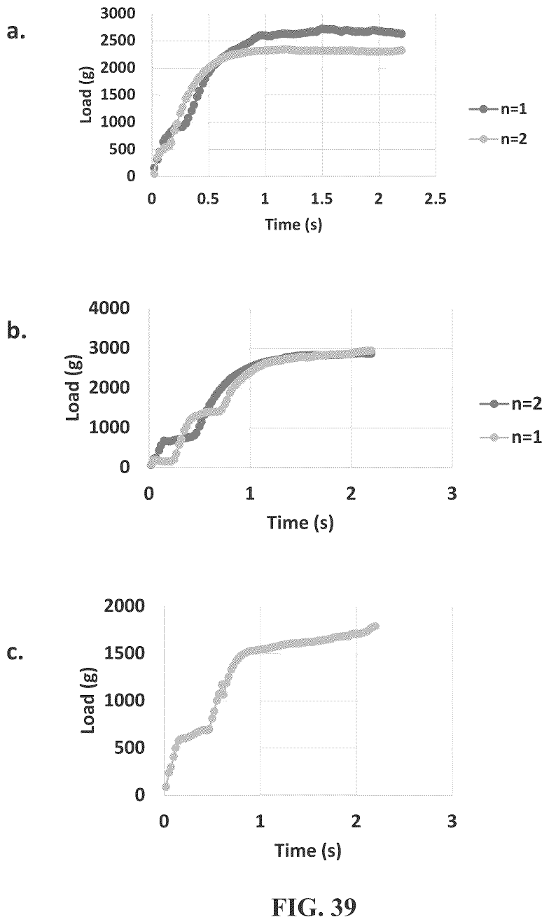

FIG. 39, panels a-c depict graphs of the syringe force profiles for each of formulations 12, 13 and 16. Panel a depicts the syringe force profile for formulation 12 (benzyl benzoate as liquid carrier). Panel b depicts the syringe force profile for formulation 13 (Miglyol 840 as liquid carrier). Panel c depicts the syringe force profile for formulation 16 (ethyl oleate as liquid carrier).

Table 14 shows the average glide force results for formulations 12, 13, 16 compared to the same measurement for BSA and MoNi suspensions (460 mg/mL) in various other liquid carriers prepared in prefilled syringes and stored for 48 hours at ambient temperature away from light.

TABLE 14
Average Glide Force for BSA + MoNi
Suspensions in Various Liquid Carriers

| | | Avg Glide Force |
|---|---|---|
| Sample | Liquid Carrier | (N), n = 2 |
| BSA + | triacetin | 44.2 |
| MoNi‘460 | triacetin:benzyl benzoate (75:25) | 37.0 |
| mg/mL’ | triacetin:benzyl benzoate (50:50) | 32.6 |
| suspension | triacetin:benzyl alcohol (75:25) | 29.6 |
| | benzyl benzoate:DMAc (75:25) | 57.2 |
| | triacetin:ethanol (75:25) | 65.9 |
| | 12: benzyl benzoate (100%) | 24.4 |
| | 13: Miglyol 840 (100%) | 27.8 |
| | 16: ethyl oleate (100%) | 16.8 |
| | 14: benzyl alcohol (100%) | Formed ‘solid’, >instrument |
| | | limit (10 kg, 98N) |

As seen in Table 14, formulation 16 with ethyl oleate as liquid carrier has the lowest syringe force of the formulations tested. Formulations 12 and 13 also exhibited average glide forces that were lower than the remaining suspension formulations tested.

### 6.1.18. Example 18—BSA and MoNi Suspension in a Mixture of Triacetin and DMAc

The syringe force profile of an exemplary injectable formulation including BSA and MoNi suspensions (460 mg/mL) in triacetin:DMAc (75:25) as liquid carrier was investigated. The formulation in triacetin: DMAc (75:25) was prepared according to general method B (as described herein). Table 15 below summarizes the formulation prepared for this example:

TABLE 15
BSA + MoNi Formulation in triacetin:DMAc

| | | | | | Vol. of | Final |
|---|---|---|---|---|---|---|
| | | | BSA + | | liquid | Conc. |
| | BSA | Polymer | MoNi | Liquid | carrier | of BSA |
| # | (mg) | Additive | (mg) | Carrier | (μL) | (mg/mL)* |
| 17 | 386.0 | MoNi | 405.4 | triacetin:DMAc | 434 | 460 |
| | | | | (75:25) | | |

*calculated using 1 g/mL as the density of spray dried powder.

Formulation 17, after loading into prefilled syringes in duplicate (n=2) was evaluated to establish a syringe force profile. Prefilled syringes (0.3 mL) were subjected to force measurements, and glide force (N) was calculated over 1-2.2 s duration.

The result of the syringe force experiment (n=2, average of the two samples tested) is shown in Table 16.

TABLE 16
Formulation 17 Syringe Force Profile Result

| Sample # | Liquid Carrier | Avg Glide Force (N), n = 2 |
|---|---|---|
| 17 | triacetin:DMAc (75:25) | 18.7 |

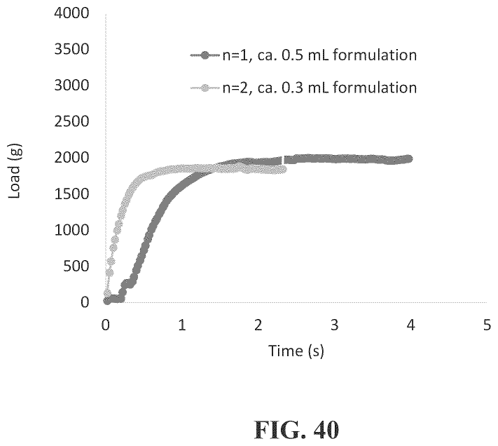

FIG. 40, is a graph showing the syringe force profile for formulation 17.

### 7. EQUIVALENTS AND INCORPORATION BY REFERENCE

While the various embodiments of the present disclosure have been particularly shown and described with reference to a preferred embodiment and various alternate embodiments, it will be understood by persons skilled in the relevant art that various changes in form and details can be made herein without departing from the spirit and scope of the disclosure.

All publications, patents, patent applications, and other documents cited in this application, including U.S. Provisional Appl. Nos. 63/437,239 and 63/532,820 and International Publication Nos. WO 2021/211976 and WO 2023/230046, are hereby incorporated by reference in their entireties for all purposes to the same extent as if each individual publication, patent, patent application, or other document were individually indicated to be incorporated by reference for all purposes.

1. An injectable pharmaceutical composition comprising:
particles, the particles comprising:
a biopharmaceutical agent; and
a polyacrylamide-based copolymer; and
a liquid carrier in which the particles are suspended.

2. The injectable pharmaceutical composition of claim 1, wherein the particles have a mean diameter of 100 microns or less.

3-6. (canceled)

7. The injectable pharmaceutical composition of claim 1, further comprising one or more of stabilizing agent, preservative, filler, bulking agent, sugar, polysaccharide, or viscosity modifier.

8-9. (canceled)

10. The injectable pharmaceutical composition of claim 1, wherein the biopharmaceutical agent is a polypeptide susceptible to aggregation in an aqueous medium.

11. (canceled)

12. The injectable pharmaceutical composition of claim 10, wherein the polypeptide is selected from antibodies and fragments thereof, cytokines, chemokines, hormones, vaccine antigens, cancer antigens, adjuvants, and combinations thereof.

13. The injectable pharmaceutical composition of claim 1, wherein the biopharmaceutical agent polypeptide is a protein.

14. The injectable pharmaceutical composition of claim 13, wherein the protein is an antibody or a fragment thereof.

15. The injectable pharmaceutical composition of claim 14, wherein the protein is a monoclonal antibody, a polyclonal antibody, an immunoglobulin G (IgG) antibody, an IgA antibody, an IgM antibody, a Fc fusion protein, or a fragment thereof.

16. The injectable pharmaceutical composition of claim 1, wherein;
the biopharmaceutical agent is a hormone or analog thereof;
the biopharmaceutical agent is insulin or an analog thereof; or
the biopharmaceutical agent is selected from glucagon, GLP-1 receptor agonist, amylin, and analogs thereof.

17-18. (canceled)

19. The injectable pharmaceutical composition of claim 1, wherein the composition comprises 0.1 to 20% by weight of the biopharmaceutical agent, wherein:
the biopharmaceutical agent is a peptide; or
the biopharmaceutical agent is insulin or an analog thereof.

20-23. (canceled)

24. The injectable pharmaceutical composition of claim 1, wherein the composition comprises 20% to 80% by weight of the biopharmaceutical agent and/or the particles comprise 75% or more by weight of the biopharmaceutical agent, wherein the biopharmaceutical agent is an antibody or fragment thereof, or a Fc fusion protein.

25-26. (canceled)

27. The injectable pharmaceutical composition of claim 1, wherein the composition comprises 0.01 to 20 wt % of the polyacrylamide-based copolymer and/or the particles comprise 0.01 to 25 wt % of the polyacrylamide-based copolymer.

28-32. (canceled)

33. The injectable pharmaceutical composition of claim 1, wherein weight to weight ratio of the polyacrylamide-based copolymer to the biopharmaceutical agent is 1:500 to 2:1, 1:50 to 1:1, or 1:25 to 1:10.

34-35. (canceled)

36. The injectable pharmaceutical composition of claim 1, wherein the polyacrylamide-based copolymer comprises:
a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof; and
a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof.

37. The injectable pharmaceutical composition of claim 36, wherein the water-soluble carrier monomer is selected from MORPH, MPAM, and combinations thereof.

38. The injectable pharmaceutical composition of claim 37, wherein the water-soluble carrier monomer comprises MORPH.

39. The injectable pharmaceutical composition of claim 37, wherein the water-soluble carrier monomer comprises MPAM.

40. The injectable pharmaceutical composition of claim 36, wherein the functional dopant monomer is selected from AMP, TMA, TBA, PHE, and combinations thereof.

41. The injectable pharmaceutical composition of claim 36, wherein the functional dopant monomer is selected from DEA, PHE, NIP, and combinations thereof.

42. The injectable pharmaceutical composition of claim 36, wherein the functional dopant monomer comprises TRI.

43. The injectable pharmaceutical composition of claim 36, wherein the functional dopant monomer comprises PHE.

44. The injectable pharmaceutical composition of claim 36, wherein the functional dopant monomer comprises NIP.

45. The injectable pharmaceutical composition of claim 36, wherein the functional dopant monomer comprises DEA.

46. The injectable pharmaceutical composition of claim 36, wherein:
the water-soluble carrier monomer is selected from MPAM, MORPH, and combinations thereof; and
the functional dopant monomer is selected from NIP, PHE, and combinations thereof.

47. The injectable pharmaceutical composition of claim 36, wherein:
the water-soluble carrier monomer is selected from MPAM, MORPH, and combinations thereof; and
the functional dopant monomer is selected from AMP, TMA, TBA, PHE, and combinations thereof.

48. The injectable pharmaceutical composition of claim 36, wherein the water-soluble carrier monomer is MPAM, and the functional dopant monomer is PHE.

49. The injectable pharmaceutical composition of claim 36, wherein the water-soluble carrier monomer is MORPH, and the functional dopant monomer is PHE.

50. The injectable pharmaceutical composition of claim 36, wherein the water-soluble carrier monomer is MORPH, and the functional dopant monomer is NIP.

51. The injectable pharmaceutical composition of claim 36, wherein the polyacrylamide-based copolymer comprises:
a) 70 wt % to 98 wt % of the water-soluble carrier monomer; and
2 wt % to 30 wt % of the functional dopant monomer;
b) 80 wt % to 95 wt % of the water-soluble carrier monomer; and
5 wt % to 20 wt % of the functional dopant monomer; or
c) 83 wt % to 98 wt % of the water-soluble carrier monomer; and
2 wt % to 17 wt % of the functional dopant monomer.

52-53. (canceled)

54. The injectable pharmaceutical composition of claim 36, wherein the polyacrylamide-based copolymer comprises:
a) 70 wt % to 85 wt % of MORPH; and
15 wt % to 30 wt % of NIP;
b) 74 wt % to 80 wt % of MORPH; and
20 wt % to 26 wt % of NIP; or
c) 77 wt % of MORPH; and
23 wt % of NIP.

55-56. (canceled)

57. The injectable pharmaceutical composition of claim 36, wherein the degree of polymerization of the polyacrylamide-based copolymer is 10 to 500, and/or the number-average molecular weight of the polyacrylamide-based copolymer is 1,000 g/mol to 40,000 g/mol.

58-63. (canceled)

64. The injectable pharmaceutical composition of claim 1, wherein the liquid carrier comprises a mixture of an organic solvent and an aqueous solution, or wherein the liquid carrier comprises an organic solvent, an oil, or a combination thereof.

65. (canceled)

66. The injectable pharmaceutical composition of claim 1, wherein the liquid carrier comprises one or more liquids selected from triacylglyceride, diacylglyceride, monoacylglyceride, an acetamide, alkyl alcohol, aryl alcohol, aralkyl alcohol, fatty acid, fatty acid ester, oil, alkane, perfluoroalkane, propylene glycol monoester, propylene glycol diester, butylene glycol monoester, butylene glycol diester, polyethylene glycol diester, and combinations thereof.

67. The injectable pharmaceutical composition of claim 66, wherein;
the liquid carrier comprises an aralkyl benzoate;
the liquid carrier comprises a triacylglyceride, diacylglyceride, monoacylglyceride, or a combination thereof;
the liquid carrier comprises N,N-dimethylacetamide (DMAc);
the liquid carrier comprises a fatty acid ester;
the liquid carrier comprises a propylene glycol diester and/or a butylene glycol diester; or
the liquid carrier comprises a blend of a triacylglyceride and one or more organic solvents that is less viscous than the triacylglyceride.

68. The injectable pharmaceutical composition of claim 67, wherein:
the aralkyl benzoate is benzyl benzoate;
the liquid carrier comprises a triacylglyceride that is triacetin;
the fatty acid ester is ethyl oleate;
the liquid carrier comprises Miglyol 840; and
the triacylglyceride is triacetin.

69-77. (canceled)

78. The injectable pharmaceutical composition of claim 66, wherein the liquid carrier comprises a 1:1 to 9:1 volume to volume (v/v) ratio of triacetin to one or more organic solvents that is less viscous than triacetin.

79-81. (canceled)

82. The injectable pharmaceutical composition of claim 78, wherein the organic solvent that is less viscous than triacetin is selected from an acetamide, an alkyl benzoate, an aryl benzoate, an aralkyl benzoate, an aryl alcohol, an aralkyl alcohol, and any combination thereof.

83-88. (canceled)

89. The injectable pharmaceutical composition of claim 82, wherein the organic solvent that is less viscous than triacetin comprises a combination of an acetamide and an aralkyl alcohol.

90. The injectable pharmaceutical composition of claim 89, wherein the acetamide is DMAc, and the aralkyl alcohol is benzyl alcohol, and wherein the v/v ratio of DMAc to benzyl alcohol is 3:1 to 2:1.

91-93. (canceled)

94. The injectable pharmaceutical composition of claim 64, wherein the organic solvent is a polar aprotic solvent selected from dimethyl sulfoxide (DMSO), N-methyl-2-pyrrolidone (NMP), and a mixture thereof.

95. (canceled)

96. The injectable pharmaceutical composition of claim 64, wherein the liquid carrier comprises an oil, wherein the oil is coconut oil, cottonseed oil, fish oil, grape seed oil, hazelnut oil, hydrogenated vegetable oils, lime oil, olive oil, palm seed oil, peanut oil, peppermint oil, safflower oil, sesame oil, soybean oil, sunflower oil, walnut oil, or any combination thereof.

97-98. (canceled)

99. The injectable pharmaceutical composition of claim 1, wherein the composition comprises a 1:5 to 4:1 or 1:1 to 2:1 weight to weight ratio of the liquid carrier to the particles.

100. (canceled)

101. An injectable pharmaceutical composition comprising:
particles, the particles comprising:
a biopharmaceutical agent; and
a polyacrylamide-based copolymer comprising:
a water-soluble carrier monomer selected from N-(3-methoxypropyl)acrylamide (MPAM), 4-acryloylmorpholine (MORPH), N,N-dimethylacrylamide (DMA), N-hydroxyethyl acrylamide (HEAM), acrylamide (AM), and combinations thereof; and
a functional dopant monomer selected from N-[tris(hydroxymethyl)-methyl]acrylamide (TRI), 2-acrylamido-2-methylpropane sulfonic acid (AMP), (3-acrylamidopropyl)trimethylammonium chloride (TMA), N-isopropylacrylamide (NIP), N—N-diethylacrylamide (DEA), N-tert-butylacrylamide (TBA), N-phenylacrylamide (PHE), and combinations thereof; and
a liquid carrier in which the particles are suspended, wherein the liquid carrier is selected from triacylglyceride, diacylglyceride, monoacylglyceride, an acetamide, alkyl alcohol, aryl alcohol, aralkyl alcohol, fatty acid, fatty acid ester, oil, alkane, perfluoroalkane, propylene glycol monoester, propylene glycol diester, butylene glycol monoester, butylene glycol diester, polyethylene glycol diester, and combinations thereof.

102-115. (canceled)

116. A syringe, loaded with a composition of claim 1.

117-127. (canceled)

128. A method of administering a biopharmaceutical to a subject by injection, the method comprising:
injecting a composition according to claim 1 to administer a therapeutically effective dose of the biopharmaceutical to a subject in need thereof.

129. (canceled)

130. A method of preparing an injectable pharmaceutical composition, the method comprising:
a) providing a mixture comprising:
a biopharmaceutical agent; and
a polyacrylamide-based copolymer in water;
b) removing the water from the mixture to obtain particles comprising the biopharmaceutical agent and the polyacrylamide-based copolymer; and
c) contacting a liquid carrier with the particles to form a suspension of the particles in the liquid carrier.

131-136. (canceled)
